(19) United States
(12) Patent Application Publication
(10) Pub. No.: US 2011/0224800 A1
(43) Pub. Date: Sep. 15, 2011

(54) CELL-SCAFFOLD CONSTRUCTS

(76) Inventors: John W. Ludlow, Carrboro, NC (US); Kelly I. Guthrie, Winston-Salem, NC (US); Andrew T. Bruce, Lexington, NC (US); Kenny Johnson, Mocksville, NC (US)

(21) Appl. No.: 13/043,252

(22) Filed: Mar. 8, 2011

Related U.S. Application Data
(60) Provisional application No. 61/312,045, filed on Mar. 9, 2010.

(51) Int. Cl. A61F 2/04

(52) U.S. Cl. 623/23.65

(57) ABSTRACT

The present invention relates to the regeneration, reconstruction, augmentation or replacement of organs or tissue structures using scaffolds and cells derived from peritoneal tissue.

### REFERENCE TO RELATED APPLICATIONS

This application claims priority under 35 U.S.C. §119(e) to U.S. provisional application No. 61/312,045, filed Mar. 9, 2010, the entire contents of which is incorporated herein by reference.

### FIELD OF THE INVENTION

The present invention relates to the regeneration, reconstruction, augmentation or replacement of laminarly organized luminal organs or tissue structures using scaffolds seeded with cells obtained from peritoneal tissue sources.

### BACKGROUND OF THE INVENTION

Several anomalies can cause the bladder to develop abnormally and require surgical augmentation. Conditions such as posterior urethral valves, bilateral ectopic ureters, bladder extrophy, cloacal extrophy, and spina bifida (ie, myelomeningocele) may cause the bladder to be noncompliant, resulting in a small capacity bladder that generates high pressures. Clinically this causes patients to suffer from incontinence while increasing their risk for renal failure due to the high pressures in the genitourinary system. The current standard of therapy for these pediatric patients is bladder augmentation through enterocystoplasty (Lewis et al. Br. J. Urol. (1990); 65:488-491). Bladder augmentation involves the removal of a section of large bowel from the patient who then has that tissue connected to the existing bladder to increase compliance, decrease pressure, and improve capacity. The surgeries are relatively complex and expensive. Even in patients with a good technical result, the procedure is associated with numerous immediate risks and chronic complications. The invasiveness, cost, and complications of these surgeries limit their use to only the most severe bladder deficiencies. A similar surgical procedure is performed in adults who require a bladder replacement, many as a result of bladder cancer. In adults, the entire bladder is resected and replaced with large bowel. Despite the risk of adverse effects, there are approximately 10,000 of these procedures performed per year in the United States, including about 10% in children with congenital abnormalities and 90% in adults with acquired disorders such as bladder cancer. There is clearly a compelling medical need for an improved approach that would eliminate or at least substantially reduce the adverse effects associated with the current standard of care.

The human urinary bladder is a musculomembranous sac, situated in the anterior part of the pelvic cavity that serves as a reservoir for urine, which it receives through the ureters and discharges through the urethra. In a human the bladder is found in the pelvis behind the pelvic bone (pubic symphysis) and is above and posteriorly connected to a drainage tube, called the urethra, that exits to the outside of the body. The urinary bladder is subject to numerous maladies and injuries which cause deterioration of the urinary bladder in patients. For example, bladder deterioration may result from infectious diseases, neoplasms and developmental abnormalities. Further, bladder deterioration may also occur as a result of trauma such as, for example, car accidents and sports injury. Urinary diversions are often necessary in bladder cancer patients. There are over 54,000 new bladder cancer cases each year in the United States of America. Most bladder cancers are of epithelial origin, and worldwide, there are approximately 336,000 new cases of urothelial carcinomas (transitional cell carcinomas (TCC)) annually (Kakizoe (2006) Cancer Sci. 97(9) 821).

Urinary diversion is a way to route and excrete urine from the body when an individual is unable to urinate due to a damaged or non-functional urinary system. In general, any condition that blocks the flow of urine and increases pressure in the ureters and/or kidneys may require a urinary diversion. Some common indications for diversion include cancer of the bladder requiring a cystectomy, a neurogenic bladder that impact renal function, radiation injury to the bladder, intractable incontinence that occurs in women, and chronic pelvic pain syndromes. In general, two major strategies exist for urinary diversion: a urostomy and a continent diversion. A urostomy involves the creation of a stoma in the abdomen which is connected to a conduit inside the body such as a short segment of the small intestine submucosa (SI) such as the ileum, colon or jejunum. In this procedure, the other end of the short SI is connected to the ureters which normally carry urine from the kidney to the bladder. Urine flows through the ureters into the short SI and out the stoma to an external collection reservoir. An alternative of this procedure is the attach the ureters directly to a stoma, also called a ureterostomy. A continent diversion involves the creation of a pouch or reservoir inside the body from a section of the stomach or small or large intestine and the use of a stoma may or may not be required. For example, a continent cutaneous reservoir may be created by obtaining a segment of the bowel and modifying it into a more spherical shape. One end of the modified segment is connected to the ureters and the other to a stoma that leads to an external collection reservoir. Finally, an orthotopic diversion may created by placing the re-shaped segment in place of the original bladder by connecting one end to the ureters and the other end to the urethra so the individual may urinate through the urethra instead of through a stoma.

Although small intestinal submucosa (SI) may be used for urinary diversion, it has been reported that the removal of the mucosa and submucosa may lead to retraction of the intestinal segment (see, e.g., Atala, A., J. Urol. 156:338 (1996)). Other problems have been reported with the use of certain gastrointestinal segments for bladder surgery including stone formation, increased mucus production, neoplasia, infection, metabolic disturbances, long term contracture and resorption. The use of natural materials for urinary diversion has shown that bladder tissue, with its specific muscular elastic properties and urothelial impermeability functions, cannot be easily replaced. In addition, the use of a patient's own bowel segments for urinary diversion requires at least two different surgical procedures where a first surgery is performed to remove a segment and a second surgery to install the urinary diversion. The requirement of multiple surgeries increases the overall cost of the procedures, the risk to the patient, and patient's overall comfort.

Therefore, due to the multiple complications associated with the use of gastrointestinal segments for urinary diversion and requirement for multiple surgical procedures, there exists a need for methods and devices for providing urinary diversion systems to patients in need of such a system.

Urinary incontinence is a prevalent problem that affects people of all ages and levels of physical health, both in the community at large and in healthcare settings. Medically, urinary incontinence predisposes a patient to urinary tract infections, pressure ulcers, perineal rashes, and urosepsis. Socially and psychologically, urinary incontinence is associated with embarrassment, social stigmatization, depression, and especially for the elderly, an increased risk of institutionalization (Herzo et al., Ann. Rev. Gerontol. Geriatrics, 9:74 (1989)). Economically, the costs are astounding; in the United States alone, over ten billion dollars per year is spent managing incontinence.

Incontinence can be attributed to genuine urinary stress (bladder and urethra hypermobility), to intrinsic sphincter deficiency (“ISD”), or both. It is especially prevalent in women, and to a lesser extent incontinence is present in children (in particular, ISD), and in men following radical prostatectomy.

Stress incontinence is an involuntary loss of urine that occurs during physical activities which increase intra-abdominal pressure, such as coughing, sneezing, laughing, or exercise. A person can suffer from one or both types of incontinence, and when suffering from both, it is called mixed incontinence. Despite all of the knowledge associated with incontinence, the majority of cases of urge incontinence are idiopathic, which means a specific cause cannot be identified. Urge incontinence may occur in anyone at any age, and it is more common in women and the elderly.

The detrusor is the bladder wall muscle that contracts to expel the urine from the bladder. Consequences of detrusor malfunction such as hyperreflexia include poor bladder compliance, high intravesical pressure, and reduction in bladder capacity, all of which may result in deterioration of the upper urinary tract.

One current treatment for urge incontinence is injection of neurotoxins, such as botulinum toxin, e.g., Botox®. It is thought that botulinum toxin exerts its effect on bladder hyperactivity by paralyzing the detrusor muscle in the bladder wall or possibly impacting afferent pathways in the bladder and reducing sensory receptors in suburothelial nerves. The large size of the botulinum toxin molecule can limit its ability to diffuse, and thus prohibits it from reaching both afferent and efferent nerve fibers. As a result, current methods of administration for overactive bladder (OAB), for example, require many injections (typically 20 to 50) of botulinum toxin into the bladder muscle wall, thus increasing the number of doctor visits and associated cost of treatment. Moreover, the safety of chronic long-term impact of inhibition of sensory neurotransmitter release from bladder has not yet been determined.

Further approaches for treatment of urinary incontinence involve administration of drugs with bladder relaxant properties, with anticholinergic medications representing the mainstay of such drugs. For example, anticholinergics such as propantheline bromide, and combination smooth muscle relaxant/anticholinergics such as racemic oxybutynin and dicyclomin, have been used to treat urge incontinence. (See, e.g., A. J. Wein, Urol. Clin. N. Am., 22:557 (1995)). Often, however, such drug therapies do not achieve complete success with all classes of incontinent patients, and often results in the patient experiencing significant side effects.

Besides drug therapies, other options used by the skilled artisan prior to the present invention include the use of artificial sphincters (Lima S. V. C. et al., J. Urology, 156:622-624 (1996), Levesque P. E. et al., J. Urology, 156:625-628 (1996)), bladder neck support prosthesis (Kondo A. et al., J. Urology, 157:824-827 (1996)), injection of cross-linked collagen (Berman C. J. et al., J. Urology, 157:122-124 (1997), Perez L. M. et al., J. Urology, 156:633-636 (1996); Leonard M. P. et al., J. Urology, 156:637-640 (1996)), and injection of polytetrafluoroethylene (Perez L. M. et al., J. Urology, 156:633-636 (1996)).

A recent well known approach for the treatment of urinary incontinence associated with ISD is to subject the patient to periurethral endoscopic collagen injections. This augments the bladder muscle in an effort to reduce the likelihood of bladder leakage or stress incontinence.

Existing solutions to circumvent incontinence have well known drawbacks. While endoscopically directed injections of collagen around the bladder neck has a quite high success rate in sphincter deficiency with no significant morbidity, the use of collagen can result in failures that occur after an average of two years and considerations need to be given to its cost effectiveness (Khullar V. et al., British J. Obstetrics & Gynecology, 104:96-99 (1996)). In addition, deterioration of patient continency, probably due to the migration phenomena (Perez L. M. et al.) may require repeated injections in order to restore continency (Herschorn S. et al., J. Urology, 156:1305-1309 (1996)).

The results with using collagen following radical prostatectomy for the treatment of stress urinary incontinence have also been generally disappointing (Klutke C. G. et al., J. Urology, 156:1703-1706 (1996)). Moreover, one study provides evidence that the injection of bovine dermal collagen produced specific antibodies of IgG and IgA class. (McCell and, M. and Delustro, F. , J. Urology 155, 2068-2073 (1996)). Thus, possible patient sensitization to the collagen could be expected over the time.

Despite of the limited success rate, transurethral collagen injection therapy remains an acceptable treatment for intrinsic sphincter deficiency, due to the lack other suitable alternatives.

At present, individuals who suffer from Overactive Bladder Disorders or Urge Incontinence are initially treated by physicians with non-invasive pharmaceutical medical products. However, if these non-invasive pharmaceutical products fail, physicians offer a more invasive solution.

Thus, a need exists for a minimally invasive method of enlarging an existing laminarily organized luminal organ or tissue structure, e.g., a bladder.

Tissue engineering principles have been applied to successfully provide implantable cell-seeded matrices for use in the reconstruction, repair, augmentation or replacement of laminarily organized luminal organs or tissue structures, such as a bladder, a portion of a bladder, or a bladder component. As described in Atala U.S. Pat. No. 6,576,019 (incorporated herein by reference in its entirety), cells may be derived from the patient's own tissue, including the bladder, urethra, ureter, and other urogenital tissue. However, there are challenges associated with a dependence upon the development and maintenance of cell culture systems from the primary organ site as the basic unit for developing new and healthy engineered tissues. For example, the treatment of a defective bladder poses a particular challenge regarding cell sourcing because it stands to reason that culturing bladder cells from a defective bladder will result in the cultured cells also being defective. Such cells are not a wise choice for populating an implantable neo-bladder scaffold or matrix. As such there is a need for alternative sources of cells that are suitable for seeding on implantable neo-organ/tissue structure scaffold or matrix.

As described by Jayo et al. Regen. Med. (2008) 3(5), 671-682 (hereinafter referred to as “Jayo I”), attempts to repair organs or tissue have been characterized by incomplete tissue replacement frequently with collagen deposition, and in some cases scar tissue formation. Jayo et al. also observed a more desirable outcome of tissue engineering is regeneration of the original structure and function of a tissue structure or organ. See also Jayo et al., J. Urol. (2008) 180;392-397 (hereinafter referred to as “Jayo II”). Certain molecules are believed to be associated with the regenerative process in vivo. For example, the chemokine MCP-1 is best known for its ability to recruit mononuclear cells. However, it also appears to be a potent mitogen for vascular smooth muscle cell proliferation. MCP-1 recruits circulating monocytes to the area of vessel injury, which in turn are typically transformed to macrophages that can serve as reservoirs for cytokines and growth factors. Macrophages also ingest cholesterol and oxidize lipids. Macrophages and muscle precursor cells are both believed to be targets for MCP-1 signaling. The CCR-2 receptor is the ligand for MCP-1 (CCL2) and CCR-2 deficient mice show a regeneration defect with enhanced adipogenesis/fibrosis. Sections from CCR-2 deficient mice when challenged with skeletal muscle regeneration demonstrated the following in comparison to normal mice: more interstitial space, a high number of inflammatory cells, large round swollen myofibers, more fibroblast accumulation in interstitial space, fat infiltration with collagen distribution around fat deposits, and fibrosis accompanied by calcium deposition (Warren et al. (2005), FASEB J.19:413-415; Selzman et al. (2002), Am J Physiol Heart Circ Physiol. 283(4);H1455-H1461; Shannon et al. (2007), Am. J. Cell Physiol. 292:C953-C967; Shireman et al. (2006), J. Surg. Res. 134(1):145-57. Epub 2006 Feb. 20; Amann et al. (1998), Brit. J. Urol. 82:118-121; Schecter et al. (2004), J. Leukocyte Bio1.75:1079-1085; Deonarine et al.,(2007), Transl Med. 5:11; Lumeng et al. (2007), J Clin. Invest. 117(1): 175-184).

The present invention concerns cell populations derived from peritoneal tissue sources, methods of isolating such cells, neo-organ/tissue structure scaffolds or matrices seeded with such cells (constructs) and methods of making the same, as well as methods of treating a patient in need using such neo-organ/tissue structure constructs.

### SUMMARY OF THE INVENTION

In one aspect, the present invention provides implantable constructs for the regeneration, reconstruction, augmentation, or replacement of a laminarly organized luminal organ or tissue structure in a subject in need of such treatment. In one embodiment, the implantable construct includes: a) a matrix having a first surface, wherein the matrix is shaped to conform to at least a part of a native luminal organ or tissue structure in a subject in need; and b) a peritoneal-derived cell population deposited on or in the first surface of the matrix, the matrix and cell population forming an implantable construct. In another embodiment, the cell population is a smooth muscle cell (SMC) population. In another aspect, the present invention provides methods for the reconstruction, augmentation, or replacement of a laminarly organized luminal organ or tissue structure in a subject in need of such treatment. In one embodiment, the method includes the step of implanting a construct into the subject at the site of treatment for the formation of the laminarily organized luminal organ or tissue structure. In one other aspect, the present invention provides methods of preparing an implantable construct for the reconstruction, augmentation, or replacement of a laminarly organized luminal organ or tissue structure in a subject in need of such treatment. In one embodiment, the method includes the step of providing a matrix having a first surface, wherein the matrix is shaped to conform to at least a part of a native luminal organ or tissue structure in the subject. In another embodiment, the method includes the step of depositing a peritoneal-derived cell population on or in the first surface of the matrix to form the implantable construct. In one other embodiment, the method provides an implantable construct.

In another aspect, the present invention provides implantable constructs for use as a neo-urinary conduit. In one embodiment, the construct includes a) a tubular matrix having a first surface adapted to allow the passage of fluid from a native vessel in a subject in need; and b) a peritoneal-derived cell population deposited on or in the first surface of the matrix, the matrix and cell population forming an implantable construct. In another embodiment, the cell population is a smooth muscle cell (SMC) population. The tubular matrix may have a first end. In one embodiment, the first end may be configured or shaped to contact the subject's abdominal wall. The first end may be configured or shaped for anastomosis to an opening in the subject's abdominal wall. In another embodiment, the first end may be configured such that it can be exteriorized to the subject's skin. In one other embodiment, the first end of the tubular matrix forms a stoma external to the subject upon implantation. The first end includes a stomal end extending through the subject's abdominal wall. In one embodiment, the stomal end is connected to the subject's skin. Upon implantation, the construct forms forms an epithelialized mucosa at the stomal end. The epithelialized mucosa may include a mucocutaneous region at the stomal end. In one embodiment, the epithelialized mucosa may have a vestibular region adjacent to the mucocutaneous region. The epithelialized mucosa may be characterized by an epithelium that first appears in the vestibular region and gradually increases through the mucocutaneous region towards the stomal end. In another embodiment, the epithelium is characterized by expression of an epithelial cell marker. The epithelialized mucosa is equivalent to a naturally-occurring mucocutaneous region.

In yet another embodiment, the tubular matrix further includes a first side opening for connection to the native vessel. The native vessel may be a first ureter. In one embodiment, the tubular matrix further includes a second end shaped for connection to a second ureter. The tubular matrix may further include a second side opening shaped for connection to a second ureter. In another embodiment, the construct is shaped to allow the passage of fluid from the the first and/or second side opening to the interior of the tubular matrix. In one other embodiment, the construct is further shaped to allow the passage of fluid from the interior of the tubular matrix to the exterior through the first end of the tubular matrix. Upon implantation, the construct allows for the passage of urine from the first and/or second ureter to the interior of the tubular matrix upon implantation. In addition, the construct allows for the passage of urine out of the subject from the interior of the matrix upon implantation. In one other aspect, the present invention provides methods of providing an implantable construct for a defective bladder in need of such treatment. In one embodiment, the method includes the step of implanting a construct described herein. In another aspect, the present invention provides methods of preparing an implantable construct for the reconstruction, augmentation, or replacement of a laminarly organized luminal organ or tissue structure in a subject in need of such treatment. In one embodiment, the method includes the step of providing a matrix having a first surface, wherein the matrix is shaped to conform to at least a part of a native luminal organ or tissue structure in the subject. In another embodiment, the method further includes the step of depositing a peritoneal-derived cell population on or in said first surface of the matrix to form said implantable construct.

In another aspect, the implantable constructs described herein include a smooth muscle cell population but are free of additional cell populations. In one embodiment, the constructs are free of urothelial cells.

### BRIEF DESCRIPTION OF THE DRAWINGS

FIG. 1A-D shows examples of bladder augmentation scaffolds.

FIG. 2A-D shows examples of bladder replacement scaffolds.

FIG. 3A shows an example of a urinary diversion or conduit scaffold. FIG. 3B-C shows an example of a urinary diversion construct having different types of cross-sectional areas, as well as potential positions for openings that may be configured to connect to ureter(s). FIG. 3C illustrates variations of a urinary diversion construct (A: open claim ovoid; B: open claim ovoid receptacle; C: closed ovoid receptacle and three tubes).

FIG. 4 shows different applications of a urinary diversion or conduit construct.

FIG. 5A-B show examples of a muscle equivalent scaffold.

FIG. 6 depicts images of various muscle equivalent scaffolds in the form of patches or strips.

FIG. 7 depicts different muscle equivalent scaffolds and representative methods of implantation. FIG. 7A depicts formation of a flat sheet of scaffold. FIG. 7B depicts a laparoscopically-suited scaffold which can be rolled at the time of implantation and fed through a laparoscopic tube and unrolled in the abdominal cavity. FIG. 7C depicts formation of a laparoscopically-suited scaffold sheet in a rolled configuration to facilitate insertion through a laparoscopic tube, after which it is unrolled in the abdominal cavity. FIG. 7D depicts formation of a laparoscopically-suited scaffold sheet in a folded configuration or accordion style to facilitate insertion through the tube, after which it is unfolded in the abdominal cavity. FIG. 7E depicts possible surgical methods for the implantation of a muscle equivalent scaffold. FIG. 7F depicts implantation sites on an empty and full bladder. FIG. 7G depicts a urinary bladder model with surgical slit showing ellipsoid created upon sectioning of surface.

FIG. 8 depicts a pre-folded accordion style scaffold sheet to facilitate insertion through a laparoscope port.

FIG. 9A depicts scaffold pre-cut into strips, then sutured together to allow stacking and insertion into the laparoscope port and secured in place in the abdominal cavity. FIG. 9B depicts one scaffold of 18.7 cm in length by 2.0 cm in width having 2 folds. FIG. 9C depicts one scaffold of 13.3 cm in length by 2.8 cm in width having 3 folds. FIG. 9D depicts one scaffold of 9.7 cm in length by 4.0 cm in width having 4 folds. FIG. 9E depicts one scaffold comprised of two pieces, 2 folds each, of 9.7 cm in length and 2.0 cm in width.

FIG. 10 shows an example of a configuration for an implanted conduit construct.

FIG. 11A depicts an exemplary configuration for a Neo-Urinary Conduit scaffold. FIG. 11B depicts two alternative configurations (A and B) for an implanted Neo-Urinary Conduit scaffold.

FIG. 12 shows an example of the implanted components of a permanent urinary diversion construct.

FIG. 13 depicts other applications of the urinary diversion constructs.

FIG. 14 depicts steps of a representative protocol for cell isolation from peritoneal tissue.

FIG. 15 shows cell morphology of canine- and porcine-derived cells.

FIG. 16A-B shows the expression of smooth muscle alpha-actin and calponin in canine-derived bladder cells. FIG. 16C-D shows the expression of smooth muscle alpha-actin and calponin in canine-derived omentum cells.

FIG. 17A-C show phenotype by FACS of canine omentum-derived cells SMC, epithelial and endothelial antigenic markers.

FIG. 18A-C show phenotype by FACS of canine omentum-derived cells SMC, epithelial and endothelial antigenic markers.

FIG. 19 shows immuno-fluorescence analysis of smooth muscle cell associated markers from omentum- and bladder-derived canine SMCs.

FIGS. 20A-B show immuno-fluorescence analysis of canine omentum-derived cells showing epithelial, endothelial and SMC antigenic markers.

FIG. 21 shows immuno-fluorescence analysis of smooth muscle cell associated markers from omentum- and bladder-derived porcine SMCs.

FIG. 22 depicts SMC gene expression by canine-derived cells by PCR.

FIG. 23 depicts SMC gene expression by canine-derived cells by PCR.

FIG. 24 depicts SMC gene expression by porcine-derived cells by PCR.

FIG. 25 shows contractile phenotype of canine-derived omentum cells.

FIG. 26 shows immuno-fluorescence analysis of canine omentum-derived cells SMC inside scaffold.

FIG. 27 depicts MCP1 protein secretion from omentum-derived SMC inscide scaffold.

FIG. 28 shows immuno-fluorescence analysis of ECM production by omentum-derived cells in scaffold.

FIG. 29 depicts omentum-derived SMC metabolism inside scaffold.

FIG. 30 depicts characteristics of a Neo-Urinary Conduit following implantation.

### DETAILED DESCRIPTION OF THE INVENTION

The present invention relates to the regeneration, reconstruction, augmentation or replacement of laminarly organized luminal organs or tissue structures in a subject in need using scaffolds seeded with cells obtained from peritoneal tissue sources. In another aspect, the present invention provides implantable constructs for use as a neo-urinary conduit that contain cells obtained from peritoneal tissue sources.

The present invention concerns cell populations derived from a peritoneal tissue source, methods of isolating such cells, neo-organ/tissue structure scaffolds or matrices seeded with such cells (constructs) and methods of making the same, and methods of treating a patient in need using such neo-organ/tissue structure constructs. The constructs of the present invention may be used for the reconstruction, augmentation or replacement, or regeneration of an organ or tissue structure as described herein.

### 1. Definitions

Unless defined otherwise, technical and scientific terms used herein have the same meaning as commonly understood by one of ordinary skill in the art to which this invention belongs. Principles of Tissue Engineering, 3^{rd} Ed. (Edited by R Lanza, R Langer, & J Vacanti), 2007 provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “smooth muscle cell” or “SMC” as used herein refers to a contractile cell that is derived from a source that is different from the native organs or tissues that are the subject of the reconstruction, augmentation or replacement constructs and methods as described herein. The smooth muscle cells provided by the present invention, once seeded and cultured on the scaffolds or matrices described herein, are capable of forming the non-striated muscle that is found in the walls of hollow organs (e.g. bladder, abdominal cavity, gastrointestinal tract, etc.) and characterized by the ability to contract and relax. Those of ordinary skill in the art will appreciate other attributes of smooth muscle cells.

The term “cell population” as used herein refers to a number of cells obtained by isolation directly from a suitable mammalian tissue source and subsequent culturing in vitro. Those of ordinary skill in the art will appreciate that various methods for isolating and culturing cell populations for use with the present invention and the various numbers of cells in a cell population that are suitable for use in the present invention and the various numbers of cells in a cell population that are suitable for use in the present invention. The cell population may be a smooth muscle cell population (SMC) derived from peritoneal tissue. The peritoneal tissue may be omentum tissue. The SMC population may be characterized by the expression of markers associated with smooth muscle cells. The SMC population may also be a purified cell population. The SMC population may be derived from an autologous or non-autologous source.

The term “autologous” refers to derived or transferred from the same individual's body. An autologous smooth muscle cell population is derived from the subject who will be recipient of an implantable construct as described herein.

The term “non-autologous” refers to derived or transferred from a donor who will not be the recipient of an implantable construct as described herein. Such non-autologous sources include sources that are allogeneic, syngeneic (autogeneic or isogeneic), and any combination thereof.

The term “marker” or “biomarker” refers generally to a DNA, RNA, protein, carbohydrate, or glycolipid-based molecular marker, the expression or presence of which in a cultured cell population can be detected by standard methods (or methods disclosed herein) and is consistent with one or more cells in the cultured cell population being a particular type of cell. In general, the term cell “marker” or “biomarker” refers to a molecule expressed in a cell population described herein that is typically expressed by a native cell. The marker may be a polypeptide expressed by the cell or an identifiable physical location on a chromosome, such as a gene, a restriction endonuclease recognition site or a nucleic acid encoding a polypeptide (e.g., an mRNA) expressed by the native cell. The marker may be an expressed region of a gene referred to as a “gene expression marker”, or some segment of DNA with no known coding function.

The term “smooth muscle cell marker” refers to generally to a DNA, RNA, protein, carbohydrate, or glycolipid-based molecular marker, the expression or presence of which in a cultured cell population can be detected by standard methods (or methods disclosed herein) and is consistent with one or more cells in the cultured cell population being a smooth muscle cell. In general, the term smooth muscle cell (SMC) “marker” or “biomarker” refers to a molecule that is typically expressed by a native smooth muscle cell. The marker may be a polypeptide expressed by the cell or an identifiable physical location on a chromosome, such as a gene, a restriction endonuclease recognition site or a nucleic acid encoding a polypeptide expressed by the SMC. The marker may be an expressed region of a gene referred to as a “gene expression marker”, or some segment of DNA with no known coding function. Such markers contemplated by the present invention include, but are not limited to, one or more of the following: myocardin, alpha-smooth muscle actin, calponin, myosin heavy chain, BAALC, desmin, myofibroblast antigen, SM22, and any combination thereof.

The terms “differentially expressed gene”, “differential gene expression” and their synonyms, which are used interchangeably, refer to a gene whose expression is activated to a higher or lower level in a first cell or cell population, relative to its expression in a second cell or cell population. The terms also include genes whose expression is activated to a higher or lower level at different stages over time during passage of the first or second cell in culture. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between the first cell and the second cell. Differential expression includes both quantitative, as well as qualitative, differences in the temporal or cellular expression pattern in a gene or its expression products among, for example, the first cell and the second cell. For the purpose of this invention, “differential gene expression” is considered to be present when there is an at least about one-fold, at least about 1.5-fold, at least about 2-fold, at least about 2.5-fold, at least about 3-fold, at least about 3.5 fold, at least about 4-fold, at least about 4.5-fold, at least about 5-fold, at least about 5.5-fold, at least about 6-fold, at least about 7-fold, at least about 8-fold, at least about 9-fold, at least about 10-fold, at least about 10.5-fold, at least about 11-fold, at least about 11.5-fold, at least about 12-fold, at least about 12.5-fold, at least about 13-fold, at least about 13.5-fold, at least about 14-fold, at least about 14.5-fold, or at least about 15-fold difference between the expression of a given gene in the first cell and the second cell, or at different stages over time during passage of the cells in culture.

The terms “inhibit”, “down-regulate”, “under-express” and “reduce” are used interchangeably and mean that the expression of a gene, or level of RNA molecules or equivalent RNA molecules encoding one or more proteins or protein subunits, or activity of one or more proteins or protein subunits, is reduced relative to one or more controls, such as, for example, one or more positive and/or negative controls.

The term “up-regulate” or “over-express” is used to mean that the expression of a gene, or level of RNA molecules or equivalent RNA molecules encoding one or more proteins or protein subunits, or activity of one or more proteins or protein subunits, is elevated relative to one or more controls, such as, for example, one or more positive and/or negative controls.

The term “contractile function” refers to smooth muscle contractile function involving the interaction of sliding actin and myosin filaments, which is initiated by calcium-activated phosphorylation of myosin thus making contraction dependent on intracellular calcium levels.

The term “peritoneal tissue” shall generally mean tissue originating from the peritoneum including, without limitation, parietal peritoneum, visceral peritoneum, and omentum. The peritoneal tissue may be in intimate contact with internal organs including, without limitation, the stomach, liver, and/or intestines. Omentum tissue may be obtained from different sources including, without limitation, the greater omentum and the lesser omentum. Omentum tissue can be obtained via an incision and a biopsy.

The term “construct” refers to at least one cell population deposited on or in a surface of a scaffold or matrix made up of one or more synthetic or naturally-occurring biocompatible materials. The cell population may be combined with a scaffold or matrix in vitro or in vivo.

The term “sample” or “patient sample” or “biological sample” shall generally mean any biological sample obtained from an individual, body fluid, body tissue, cell line, tissue culture, or other source. The term includes body fluids such as, for example, blood such as peripheral blood or venous blood, urine and other liquid samples of biological origin, such as lipoaspirates, and solid tissue biopsies such as a biopsy specimen (e.g., peritoneal tissue biopsy), or tissue cultures or cells derived therefrom, and the progeny thereof. The definition also includes samples that have been manipulated in any way after they are obtained from a source, such as by treatment with reagents, solubilization, or enrichment for certain components, such as proteins or polynucleotides. The definition also encompasses a clinical sample, and also includes cells in culture, cell supernatants, cell lysates, serum, plasma, biological fluid, and tissue samples. The source of a sample may be solid tissue, such as from fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in the development of the subject. The biological sample may contain compounds which are not naturally present with or in the tissue in nature such as preservatives, anticoagulants, buffers, fixatives, nutrients, antibiotics, or the like. The sample can be used for a diagnostic or monitoring assay. Methods for obtaining samples from mammals are well known in the art. If the term “sample” is used alone, it shall still mean that the “sample” is a “biological sample” or “patient sample”, i.e., the terms are used interchangeably. A sample may also be a test sample.

The term “test sample” refers to a sample from a subject following implantation of a construct described herein. The test sample may originate from various sources in the mammalian subject including, without limitation, blood, serum, urine, semen, bone marrow, mucosa, tissue, etc.

The term “control” or “control sample” refers a negative control in which a negative result is expected to help correlate a positive result in the test sample. Alternatively, the control may be a positive control in which a positive result is expected to help correlate a negative result in the test sample. Controls that are suitable for the present invention include, without limitation, a sample known to have normal levels of a cytokine, a sample obtained from a mammalian subject known not to have been implanted with a construct described herein, and a sample obtained from a mammalian subject known to be normal. A control may also be a sample obtained from a subject prior to implantation of a construct described herein. In addition, the control may be a sample containing normal cells that have the same origin as cells contained in the test sample. Those of skill in the art will appreciate other controls suitable for use in the present invention.

The term “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “subject” shall mean any single human subject, including a patient, eligible for treatment, who is experiencing or has experienced one or more signs, symptoms, or other indicators of a deficient organ function or failure, including a deficient, damaged or non-functional urinary system. Such subjects include, without limitation, subjects who are newly diagnosed or previously diagnosed and now experiencing a recurrence or relapse, or are at risk for deficient organ function or failure, no matter the cause. The subject may have been previously treated for a condition associate with deficient organ function or failure, or not so treated. Subjects may be candidates for a urinary diversion including, without limitation, subjects having cancer of the bladder requiring a cystectomy, subjects having a neurogenic bladder that impacts renal function, subjects having radiation injury to the bladder, and subjects having intractable incontinence. The subject may be newly diagnosed as requiring a urinary diversion, or previously diagnosed as requiring a urinary diversion and now experiencing complications, or at risk for a deficient, damaged or non-functional urinary system, no matter the cause. The subject may have been previously treated for a condition associated with a deficient, damaged or non-functional urinary system, or not so treated.

The term “urinary diversion” or “conduit” refers to the resulting organ or tissue structure resulting from the subject's interaction over time with an implanted urinary diversion construct, anastomosed ureters, and optionally an adjacent atrium. The atrium is the anterior connecting chamber that allows for urine passage through the abdominal wall and may be made by the most anterior tube-like portion of a peritoneal wrap connecting the caudal end of the construct (located in the intra-abdominal cavity) to the skin.

The terms “caudal” and “cranial” are descriptive terms relating to the urinary production and flow. The term “caudal” refers to the end of the urinary diversion construct that upon implantation is closest to the stoma, while the term “cranial” refers to the end of the urinary diversion construct that upon implantation is closest to the kidneys and ureters.

The term “detritis” refers to debris formed during the healing and regenerative process that occurs following implantation of a urinary diversion construct. Detritis can be made up of exfoliated tissue cells, inflammatory exudate and scaffold biodegradation. If the conduit is obstructed (improper outflow) by such debris, then the stagnated debris forms a detritis or semisolid bolus within the lumen of the conduit.

The term “debridement” refers to surgical or non-surgical removal of foreign matter, or lacerated, devitalized, contaminated or dead tissue from a conduit in order to prevent infection, prevent obstruction, and to promote the healing process. The debridement may involved the removal of detritis.

The term “stoma” refers to a surgically created opening used to pass urine from the draining outflow end of a urinary diversion construct to outside the body. The urine is typically collected in a reservoir outside the body.

The term “stoma port” or “stoma button” refers to means, such as a device used to maintain the integrity of the stoma opening.

The term “expanding” or “enlarging” as used herein refers to increasing the size of the existing laminarily organized luminal organ or tissue structure. For example, in one aspect of the invention, the existing laminarily organized luminal organ or tissue structure may be enlarged by 10, 11, 12, 13, 14, 15, 16, 17, 18, 19, 20, 21, 22, 23, 24, 25, 26, 27, 28, or 29 percent. In another aspect of the invention, the existing laminarily organized luminal organ or tissue structure may be enlarged such as to increase the existing volumetric capacity of the existing laminarily organized luminal organ or tissue structure.

The term “volumetric capacity” as used herein refers to the amount of liquid capable of being contained in a defined area.

“Regeneration prognosis” or “regenerative prognosis” generally refers to a forecast or prediction of the probable course or outcome of the implantation of a construct described herein. As used herein, regeneration prognosis includes the forecast or prediction of any one or more of the following: development or improvement of a functional bladder after bladder replacement or augmentation, development of a functional urinary diversion after conduit implantation, development of improved bladder capacity, and development of improved bladder compliance. As used herein, “prognostic for regeneration” means providing a forecast or prediction of the probable course or outcome of the implantation of a new organ or tissue structure. In some embodiments, “prognostic for regeneration” comprises providing the forecast or prediction of (prognostic for) any one or more of the following: development or improvement of a functional bladder after bladder replacement or augmentation, development of a functional urinary diversion after conduit implantation, development of bladder capacity or improved bladder capacity, and development of bladder compliance or improved bladder compliance.

“Regenerated tissue” refers to the tissue of a new organ or tissue structure that develops after implantation of a construct as described herein. The organ or tissue structure may be a bladder or a part of a bladder. The regenerated tissue may include a continous urothelium with underlying smooth muscle.

### 2. Cell Populations

The present invention provides populations of smooth muscle cells for use in the reconstruction, augmentation or replacement of laminarly organized luminal organs or tissue structures in which the cell population comprises at least one cell that has contractile function and is positive for one or more smooth muscle cell markers.

As discussed herein, tissue engineering principles have been successfully applied to provide implantable cell-seeded matrices for use in the reconstruction, repair, augmentation or replacement of laminarily organized luminal organs and tissue structures, such as a bladder or a bladder component, typically composed of urothelial and smooth muscle layers. (Becker et al. Eur. Urol. 51, 1217-1228 (2007); Frimberger et al. Regen. Med. 1, 425-435 (2006); Roth et al. Curr. Urol. Rep. 10, 119-125 (2009); Wood et al. Curr. Opin. Urol. 18, 564-569). Smooth muscle cells may be derived from the patient's own tissue, including the bladder, urethra, ureter and other urogenital tissue. However, there are challenges associated with dependence upon the development and maintenance of cell culture systems from the primary organ site as the basic unit for developing new and healthy engineered tissues, as for example during treatment of cancerous bladder tissue. Clearly, such cancerous cells are inappropriate for populating an implantable neo-bladder scaffold or matrix.

The present invention provides cell populations that are derived from sources that are different from the organ or tissue structure that is the subject of the reconstruction, augmentation or replacement. In one embodiment, the source is an autologous source. In another embodiment, the source is a non-autologous source.

In another aspect, the cell population expresses markers consistent with or typical of a smooth muscle cell population.

In one other aspect, the present invention provides smooth muscle cell populations isolated from sources that are different from the luminal organ or tissue structure that is the subject of the reconstruction, augmentation or replacement. In a preferred embodiment, the luminal organ or tissue structure is a bladder or portion of a bladder.

In one aspect, the source is peritoneal tissue. In one embodiment, the peritoneal tissue-derived smooth muscle cell population is derived from a patient sample or a donor sample. The patient or donor sample may be peritoneal tissue removed during a biopsy. The peritoneal tissue may be omentum tissue

In yet one other embodiment, the isolated cell populations of the present invention, upon culturing, can develop various smooth muscle cell characteristics including, but not limited to, hill-and-valley morphology, expression of one or more smooth muscle cell markers, contractile function, filament formation, and cytokine synthesis.

In one aspect, the cultured cell population is characterized by its hill-and-valley morphology. The cells having a hill-and-valley morphology may have various characteristics including, without limitation, spindly shaped, flattened and fibroblast-like upon passage, elongated and arranged in parallel rows, a “whirled” appearance of growth, and any combination thereof. In one embodiment, the cell population upon culturing in the appropriate media develops a “hill-and-valley morphology” that is typical of cultured smooth muscle cells.

In another aspect, the cultured cell population is characterized by the presence of one or more smooth muscle cell markers. In one embodiment, the cell population upon culturing in the appropriate media develops detectable smooth muscle cell markers including, without limitation, one or more of the following: desmin, alpha-smooth muscle actin, myosin heavy chain, calponin, myocardin, vimentin, myofibroblast, BAALC, SM22, and any combination thereof.

In one aspect, the cultured cell population is characterized by the absence of one or more epithelial or endothelial markers. In one embodiment, the cell population upon culturing in the appropriate media does not develop detectable epithelial or endothelial markers including, without limitation, one or more of the following: Ulex europeas Agglutinin 1 (UEA-1), EpCam, CDH5, KDR, FLT1, PECAM, TEK, vWF, cytokeratin AE1/AE3, and any combination thereof.

In one other aspect, the cultured cell population is characterized by the presence of one or more cells having contractile function. In one embodiment, the cell population upon culturing in the appropriate media develops contractile function. In another embodiment, the contractile function is calcium dependent. In one other embodiment, the calcium-dependent contractile function is demonstrated by inhibition of contraction with a calcium chelator. In another embodiment, the calcium chelator is EDTA. Those of ordinary skill in the art will appreciate that other chelators known in the art may be suitable.

In yet another aspect, the cultured cell population is characterized by filament formation. In one embodiment, the cell population upon culturing in the appropriate media undergoes filament formation.

In one aspect, the cell population includes at least one cell expressing one or more cytokines. In one embodiment, the cytokine is MCP-1.

In one aspect, the present invention provides a regenerative cell population containing at least one regenerative cell that when deposited on a scaffold or matrix as described herein and implanted into a subject in need, provides a regenerative effect for the organ or tissue structure that is the subject of the reconstruction, augmentation, or replacement contemplated herein. A regenerative cell population has the ability to stimulate or initiate regeneration of laminarly organized luminal organs or tissue structures upon implantation into a patient in need. In general, the regeneration of an organ or tissue structure is characterized by the restoration of cellular components, tissue organization and architecture, function, and regulative development. In addition, a regenerative cell population minimizes the incompleteness or disorder that tends to occur at the implantation site of a cell-seeded luminal organ or tissue structure construct. Disorganization at the site of implantation can manifest itself as increased collagen deposition and/or scar tissue formation, each of which can be minimized through the use of a regenerative cell population. In addition, certain cellular events are indicative of the regenerative process. In the case of a regenerated bladder or portion of a bladder using the cell populations and scaffolds described herein, a regenerating organ or tissue structure is composed of a smooth muscle parenchyma with fibrovascular tissue radiating around numerous microvessels that extend toward the luminal surface, as well as stromal elements having well developed blood vessels aligned to the mucosal surface (see Jayo II supra). A regenerating bladder or portion of a bladder is also characterized by the presence of spindloid/mesenchymal cells and aSMA positive muscle precursor cells. In one embodiment, the aSMA positive spindloid cells are observed in neostromal tissues and around multiple neo-vessels (arterioles).

In one embodiment, the present invention provides a cell population that when deposited on a scaffold or matrix as described herein and implanted into a subject in need, provides a reparative effect for the organ or tissue structure that is the subject of the reconstruction, augmentation, or replacement contemplated herein. In other embodiments, a reparative effect is characterized by scar tissue formation and/or collagen deposition. Those of skill in the art will appreciate other characteristics of repair that are known in the art.

In another aspect, the regenerative cell population provides a regenerative effect characterized by the adaptive regulation of the size of a restored laminarly organized luminal organ or tissue structure. In one embodiment, the regenerative cell population's regenerative effect is the establishment of adaptive regulation that is specific to the subject that receives the scaffold or matrix seeded with the regenerative cell population. In one embodiment, the adaptive regulation is the replacement or augmentation of a bladder in a subject using a construct described herein such that the neo-bladder grows and develops to a size that is proportional to the subject's body size.

In one embodiment, the cell population capable of regenerative stimulation is an MCP-1 producing cell population, which contains at least one cell that expresses the chemokine product MCP-1. MCP-1 regenerative stimulation is characterized by the recruitment of certain cell types to the site of implantation. In one embodiment, MCP-1 recruits muscle progenitor cells to the site of implantation to proliferate within the neo-bladder. In another embodiment, MCP-1 recruits monocytes to the site of implantation which in turn produce various cytokines and/or chemokines to facilitate the regenerative process. In one other embodiment, MCP-1 induces omental cells to develop into muscle cells.

In one aspect, the present invention provides the use of specific cytokines, such as MCP-1, as a surrogate marker for tissue regeneration. Such a marker could be used in conjunction with an assessment of regeneration based on whether function has been reconstituted. Monitoring a surrogate marker over the time course of regeneration may also serve as a prognostic indicator of regeneration.

In another embodiment, the cell population is a purified cell population. A purified cell population as described herein is characterized by a phenotype based on one or more of morphology, the expression of markers, and function. The phenotype includes without limitation, one or more of hill-and-valley morphology, expression of one or more smooth muscle cell markers, expression of cytokines, a finite proliferative lifespan in culture, contractile function, and ability to induce filament formation. The phenotype may include other features described herein or known to those of ordinary skill in the art. In another embodiment, the purified populations are substantially homogeneous for a smooth muscle cell population as described herein. A purified population that is substantially homogeneous is typically at least about 90% homogeneous, as judged by one or more of morphology, the expression of markers, and function. In other embodiments, the purified populations are at least about 95% homogeneous, at least about 98% homogeneous, or at least about 99.5% homogeneous.

In all embodiments, the SMC population is derived from an autologous source or a non-autologous source.

In another aspect, the present invention contemplates the application of the SMC populations described herein for ocular disorders. An ocular disorder is one in which the subject has a defective eye due to improper function of the muscles of the eye. Smooth muscle is present as ciliary muscle in the eye and controls the eye's accommodation for viewing objects at varying distances and regulates the flow of aqueous humour through Schlemm's canal. Smooth muscle is also present in then iris of the eye. Individuals with ocular disorders such as presbyopia and hyperopia could benefit from these SMC populations. In one embodiment, an SMC cell population could be isolated from the peritoneal tissue of a subject in need or a donor. The cell population could be seeded onto a scaffold suitable for implantation at a site within the eye of the subject. An advantage of the cell populations of the present invention is that suitable SMCs may not be available for sourcing from the subject's eye if the subject has a defective eye or due to the limited availability of eye tissue. An SMC population could be isolated from a biopsy, cultured, seeded on a suitable scaffold, and implanted into the subject to provide new eye tissue. The peritoneal tissue may be omentum tissue.

In another embodiment, the smooth muscle cell populations of the present invention may be administered to a subject having an ocular disorder without the use of a scaffold, such as by engraftment. Those of ordinary skill in the art will appreciate suitable methods of engraftment.

In one aspect, the present invention concerns isolated smooth muscle cell populations derived from peritoneal tissue. In one embodiment, the peritoneal-derived cell populations contain one or more cells having contractile function, that are positive for a smooth muscle cell marker.

In all embodiments, the cell populations may be characterized by one or more smooth muscle cell markers selected from the following: myocardin, alpha-smooth muscle actin, calponin, myosin heavy chain, BAALC, desmin, myofibroblast antigen, vimentin, and SM22. In some embodiments, the cell populations may express myocardin (MYOCD). In all embodiments, the term “MYOCD” includes a nucleic acid encoding a MYOCD polypeptide and a MYOCD polypeptide. In all embodiments, the contractile function of the cell populations may be calcium-dependent.

### 3. Methods of Isolating Cell Populations

Autologous cell populations are derived directly from the subjects in need of treatment. Non-autologous cell populations are derived from donors. The source tissue is generally not the same as the organ or tissues structure that is in need of the treatment. A population of cells may be derived from the patient's own tissue or donor tissue, such as, for example, from peritoneal tissue. In one embodiment, the source tissue is omentum tissue. The cells may be isolated in biopsies. In addition, the cells may be frozen or expanded before use.

To prepare for construction of a cell-seeded scaffold, sample(s) containing smooth muscle cells are dissociated into appropriate cell suspension(s). Methods for the isolation and culture of cells were discussed in issued U.S. Pat. No. 5,567,612 which is herein specifically incorporated by reference. Dissociation of the cells to the single cell stage is not essential for the initial primary culture because single cell suspension may be reached after a period of in vitro culture. Tissue dissociation may be performed by mechanical and enzymatic disruption of the extracellular matrix and the intercellular junctions that hold the cells together. Cells can be cultured in vitro, if desired, to increase the number of cells available for seeding on a scaffold.

Cells may be transfected prior to seeding with genetic material. Smooth muscle cells could be transfected with specific genes prior to polymer seeding. The cell-polymer construct could carry genetic information required for the long term survival of the host or the tissue engineered neo-organ.

Cell cultures may be prepared with or without a cell fractionation step. Cell fractionation may be performed using techniques, which is known to those of skill in the art. Cell fractionation may be performed based on cell size, DNA content, cell surface antigens, and viability. For example, smooth muscle cells may be enriched from peritoneal tissue, while endothelial cells may be reduced for smooth muscle cell collection. While cell fractionation may be used, it is not necessary for the practice of the invention. The peritoneal tissue may be omentum tissue.

Another optional procedure in the methods described herein is cryopreservation. Cryogenic preservation may be useful, for example, to reduce the need for multiple invasive surgical procedures. Cells taken from a biopsy or sample from the subject may be amplified and a portion of the amplified cells may be used and another portion may be cryogenically preserved. The ability to amplify and preserve cells may minimize the number of surgical procedures required. Another example of the utility of cryogenic preservation is in tissue banks. Cells may be stored, for example, in a donor tissue bank. As cells are needed for new organs or tissue structures, the cryopreserved supply of cells may be used as needed. Patients who have a disease or undergoing treatment which may endanger their existing organs or tissue structures may cryogenically preserve one or more biopsies. Later, if the patient's own organ or tissue structure fails, the cryogenically preserved cells may be thawed and used for treatment. For example, if a cancer reappeared in a new organ or tissue structure after treatment, cryogenically preserved cells may be used for reconstruction of the organ or tissue structure without the need for additional biopsies.

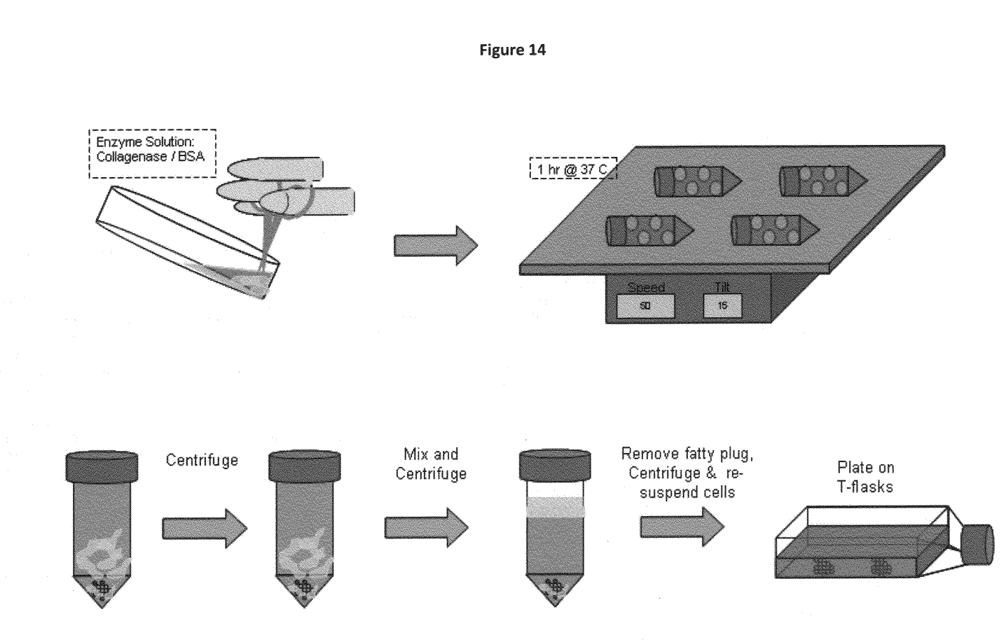

Smooth muscle cells may be isolated from peritoneal tissue based on the following general protocol. A biopsy specimen of suitable weight (e.g., in grams) and/or area (e.g., cm²) can be obtained. The following is a representative example of a protocol suitable for the isolation of smooth muscle cells from omentum tissue. A suitable gram weight of omentum tissue (e.g., 7-25 g) can be obtained by biopsy and washed with PBS (e.g., 3 times), minced with a scalpel and scissors, transferred into a 50mL conical tube and incubated at 37° C. for 60 minutes in a solution of collagenase (e.g., 0.1 to 0.3%) (Worthington) and 1% BSA in DMEM-HG. The tubes may be either continually rocked or periodically shaken to facilitate digestion. The digested sample can be pelleted by centrifugation at 600 g for 10 minutes and resuspended in DMEM-HG+10% FBS. The pellet may then be used to seed passage zero. FIG. 14 depicts a representative protocol for cell isolation from peritoneal tissue.

Those of ordinary skill in the art will appreciate additional methods for the isolation of smooth muscle cells.

In one aspect, the present invention provides methods of isolating smooth muscle cell populations from peritoneal tissue. In another aspect, the present invention provides methods for isolating an isolated smooth muscle cell population from peritoneal tissue. The peritoneal tissue may be omentum tissue. In one embodiment, the method comprises a) obtaining omentum tissue, b) digesting the omentum tissue, c) centrifuging the digested omentum tissue to pellet an SMC-containing fraction, d) culturing the pelleted fraction, and e) isolating a smooth muscle cell population from the fraction. In one embodiment, the culturing step comprises washing the pellet, re-suspending the pellet in a cell culture media, and plating the re-suspended pellet. In another embodiment, the culturing step comprises providing a cell population that is adherent to the cell culture support, such as a plate or container. In another embodiment, the method further comprises expanding the cultured cell population. In other embodiments, the method further comprises analyzing the smooth muscle cell population for smooth muscle cell characteristics. In one embodiment, the omentum tissue is derived from an autologous or non-autologous source.

In one other aspect, the present invention provides methods of isolating and culturing populations of smooth muscle cells that contain at least one cell that has contractile function and is positive for one or more smooth muscle cell markers. In one embodiment, the method includes the step of obtaining a sample from a patient in need of the reconstruction, augmentation or replacement of a laminarily organized luminal organ or tissue structure, where the sample is not obtained from the luminal organ or tissue structure that is in need of the reconstruction, augmentation or replacement. In another embodiment, smooth muscle cells are derived from the patient sample. In one other embodiment, the luminal organ or tissue structure is a bladder or portion of a bladder. In one embodiment, the sample is an autologous or non-autologous sample. In another embodiment, the sample is a peritoneal tissue sample. The peritoneal tissue sample may be an omentum tissue sample.

In another embodiment, the obtaining step is followed by a separation step.

In the case of an omentum tissue sample, the purification step includes digestion of the sample with collagenase, centrifuging the digested sample, mixing of the centrifuged sample to provide an SMC-containing fraction, centrifuging the mixed sample to obtain the fraction that can be resuspended for subsequent culturing.

In one embodiment, the culturing method includes the use of cell culture media containing minimal essential medium (e.g., DMEM or a-MEM) and fetal bovine serum (e.g., 10% FBS) by standard conditions known to those of ordinary skill in the art.

### 4. Scaffolds

As described in Atala U.S. Pat. No. 6,576,019 (incorporated herein by reference in its entirety), scaffolds or polymeric matrices may be composed of a variety of different materials. In general, biocompatible material and especially biodegradable material is the preferred material for the construction of the scaffolds described herein. The scaffolds are implantable, biocompatible, synthetic or natural polymeric matrices with at least two separate surfaces. The scaffolds are shaped to conform to a at least a part of the luminal organ or tissue structure in need or treatment. The biocompatible materials are biodegradable. Biocompatible refers to materials which do not have toxic or injurious effects on biological functions. Biodegradable refers to material that can be absorbed or degraded in a patient's body. Examples of biodegradable materials include, for example, absorbable sutures. Representative materials for forming the scaffolds include natural or synthetic polymers, such as, for example, collagen, poly(alpha hydroxy esters) such as poly(lactic acid), poly(glycolic acid), polyorthoesters and polyanhydrides and their copolymers, which degraded by hydrolysis at a controlled rate and are reabsorbed. These materials provide the maximum control of degradability, manageability, size and configuration. Preferred biodegradable polymer material include polyglycolic acid and polyglactin, developed as absorbable synthetic suture material. Polyglycolic acid and polyglactin fibers may be used as supplied by the manufacturer. Other scaffold materials include cellulose ether, cellulose, cellulosic ester, fluorinated polyethylene, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polytetrafluoroethylene, polythioether, polytriazole, polyurethane, polyvinyl, polyvinylidene fluoride, regenerated cellulose, silicone, urea-formaldehyde, or copolymers or physical blends of these materials. The material may be impregnated with suitable antimicrobial agents and may be colored by a color additive to improve visibility and to aid in surgical procedures.

Other scaffold materials that are biodegradable include synthetic suture material manufactured by Ethicon Co. (Ethicon Co., Somerville, N.J.), such as MONOCRYL™ (copolymer of glycolide and epsilon-caprolactone), VICRYL™ or Polyglactin 910 (copolymer of lactide and glycolide coated with Polyglactin 370 and calcium stearate), and PANACRYL™ (copolymer of lactide and glycolide coated with a polymer of caprolactone and glycolide). (Craig P. H., Williams J. A., Davis K. W., et al.: A Biological Comparison of Polyglactin 910 and Polyglycolic Acid Synthetic Absorbable Sutures. Surg. 141; 1010, (1975)) and polyglycolic acid. These materials can be used as supplied by the manufacturer.

In yet another embodiment, the matrix or scaffold can be created using parts of a natural decellularized organ. Biostructures, or parts of organs can be decellularized by removing the entire cellular and tissue content from the organ. The decellularization process comprises a series of sequential extractions. One key feature of this extraction process is that harsh extraction that may disturb or destroy the complex infra-structure of the biostructure, be avoided. The first step involves removal of cellular debris and solubilization of the cell membrane. This is followed by solubilization of the nuclear cytoplasmic components and the nuclear components.

Preferably, the biostructure, e.g., part of an organ is decellularized by removing the cell membrane and cellular debris surrounding the part of the organ using gentle mechanical disruption methods. The gentle mechanical disruption methods must be sufficient to disrupt the cellular membrane. However, the process of decellularization should avoid damage or disturbance of the biostructure's complex infra-structure. Gentle mechanical disruption methods include scraping the surface of the organ part, agitating the organ part, or stirring the organ in a suitable volume of fluid, e.g., distilled water. In one preferred embodiment, the gentle mechanical disruption method includes stirring the organ part in a suitable volume of distilled water until the cell membrane is disrupted and the cellular debris has been removed from the organ.

After the cell membrane has been removed, the nuclear and cytoplasmic components of the biostructure are removed. This can be performed by solubilizing the cellular and nuclear components without disrupting the infra-structure. To solubilize the nuclear components, non-ionic detergents or surfactants may be used. Examples of nonionic detergents or surfactants include, but are not limited to, the Triton series, available from Rohm and Haas of Philadelphia, Pa., which includes Triton X-100, Triton N-101, Triton X-114, Triton X-405, Triton X-705, and Triton DF-16, available commercially from many vendors; the Tween series, such as monolaurate (Tween 20), monopalmitate (Tween 40), monooleate (Tween 80), and polyoxethylene-23-lauryl ether (Brij. 35), polyoxyethylene ether W-1 (Polyox), and the like, sodium cholate, deoxycholates, CHAPS, saponin, n-Decyl-D-glucopuranoside, n-heptyl-D-glucopyranoside, n-Octyl-D-glucopyranoside and Nonidet P-40.

One skilled in the art will appreciate that a description of compounds belonging to the foregoing classifications, and vendors may be commercially obtained and may be found in “Chemical Classification, Emulsifiers and Detergents”, McCutcheon's, Emulsifiers and Detergents, 1986, North American and International Editions, McCutcheon Division, MC Publishing Co., Glen Rock, N.J., U.S.A. and Judith Neugebauer, A Guide to the Properties and Uses of Detergents in Biology and Biochemistry, Calbiochem. R., Hoechst Celanese Corp., 1987. In one preferred embodiment, the non-ionic surfactant is the Triton. series, preferably, Triton X-100.

The concentration of the non-ionic detergent may be altered depending on the type of biostructure being decellularized. For example, for delicate tissues, e.g., blood vessels, the concentration of the detergent should be decreased. Preferred concentration ranges of non-ionic detergent can be from about 0.001 to about 2.0% (w/v). More preferably, about 0.05 to about 1.0% (w/v). Even more preferably, about, 0.1% (w/v) to about 0.8% (w/v). Preferred concentrations of these range from about 0.001 to about 0.2% (w/v), with about 0.05 to about 0.1% (w/v) particular preferred.

The cytoskeletal component, which includes the dense cytoplasmic filament networks, intercellular complexes and apical microcellular structures, may be solubilized using alkaline solution, such as, ammonium hydroxide. Other alkaline solution consisting of ammonium salts or their derivatives may also be used to solubilize the cytoskeletal components. Examples of other suitable ammonium solutions include ammonium sulphate, ammonium acetate and ammonium hydroxide. In a preferred embodiment, ammonium hydroxide is used.

The concentration of the alkaline solutions, e.g., ammonium hydroxide, may be altered depending on the type of biostructure being decellularized. For example, for delicate tissues, e.g., blood vessels, the concentration of the detergent should be decreased. Preferred concentrations ranges can be from about 0.001 to about 2.0% (w/v). More preferably, about 0.005 to about 0.1% (w/v). Even more preferably, about, 0.01% (w/v) to about 0.08% (w/v).

The decellularized, lyophilized structure may be stored at a suitable temperature until required for use. Prior to use, the decellularized structure can be equilibrated in suitable isotonic buffer or cell culture medium. Suitable buffers include, but are not limited to, phosphate buffered saline (PBS), saline, MOPS, HEPES, Hank's Balanced Salt Solution, and the like. Suitable cell culture medium includes, but is not limited to, RPMI 1640, Fisher's, Iscove's, McCoy's, Dulbecco's medium, and the like.

Still other biocompatible materials that may be used include stainless steel, titanium, silicone, gold and silastic.

The polymeric matrix or scaffold can be reinforced. For example, reinforcing materials may be added during the formation of a synthetic matrix or scaffold or attached to the natural or synthetic matrix prior to implantation. Representative materials for forming the reinforcement include natural or synthetic polymers, such as, for example, collagen, poly(alpha hydroxy esters) such as poly(lactic acid), poly(glycolic acid), polyorthoesters and polyanhydrides and their copolymers, which degraded by hydrolysis at a controlled rate and are reabsorbed. These materials provide the maximum control of degradability, manageability, size and configuration.

The biodegradable polymers can be characterized with respect to mechanical properties, such as tensile strength using an Instron tester, for polymer molecular weight by gel permeation chromatography (GPC), glass, transition temperature by differential scanning calorimetry (DSC) and bond structure by infrared (IR) spectroscopy; with respect to toxicology by initial screening tests involving Ames assays and in vitro teratogenicity assays and implantation studies in animals for immunogenicity, inflammation, release and degradation studies. In vitro cell attachment and viability can be assessed using scanning electron microscopy, histology and quantitative assessment with radioisotopes. The biodegradable material may also be characterized with respect to the amount of time necessary for the material to degrade when implanted in a patient. By varying the construction, such as, for example, the thickness and mesh size, the biodegradable material may substantially biodegrade between about 2 years or about 2 months, preferably between about 18 months and about 4 months, most preferably between about 15 months and about 8 months and most preferably between about 12 months and about 10 months. If necessary, the biodegradable material may be constructed so as not to degrade substantially within about 3 years, or about 4 years or about five or more years.

The polymeric matrix or scaffold may be fabricated with controlled pore structure as described above. The size of the pores may be used to determine the cell distribution. For example, the pores on the polymeric matrix or scaffold may be large to enable cells to migrate from one surface to the opposite surface. Alternatively, the pores may be small such that there is fluid communication between the two sides of the polymeric matrix or scaffold but cells cannot pass through. Suitable pore size to accomplish this objective may be about 0.04 micron to about 10 microns in diameter, preferably between about 0.4 micron to about 4 microns in diameter. In some embodiments, a surface of the polymeric matrix or scaffold may comprise pores sufficiently large to allow attachment and migration of a cell population into the pores. The pore size may be reduced in the interior of the polymeric matrix or scaffold to prevent cells from migrating from one side of the polymeric matrix or scaffold to the opposite side. One embodiment of a polymeric matrix or scaffold with reduced pore size is a laminated structure of a small pore material sandwiched between two large pore material. Polycarbonate membranes are especially suitable because they can be fabricated in very controlled pore sizes such as, for example, about 0.01 microns, about 0.05 micron, about 0.1 micron, about 0.2 micron, about 0.45 micron, about 0.6 micron, about 1.0 micron, about 2.0 microns and about 4.0 microns. At the submicron level the polymeric matrix or scaffold may be impermeable to bacteria, viruses and other microbes.

The following characteristics or criteria, among others, are taken into account in the design of each discrete matrix, or part thereof: (i) shape, (ii) strength, (iii) stiffness and rigidity, and (iv) suturability (the degree to which the matrix, or part thereof, is readily sutured or otherwise attached to adjacent tissue). As used herein, the stiffness of a given matrix or scaffold is defined by the modulus of elasticity, a coefficient expressing the ratio between stress per unit area acting to deform the scaffold and the amount of deformation that results from it. (See e.g., Handbook of Biomaterials evaluation, Scientific, Technical, and Clinical Testing of Implant Materials, 2nd edition, edited by Andreas F. von Recum, (1999); Ratner, et al., Biomaterials Science: An Introduction to Materials in Medicine, Academic Press (1996)). The rigidity of a scaffold refers to the degree of flexibility (or lack thereof) exhibited by a given scaffold.

Each of these criteria is a variable that can be changed (through, among other things, the choice of material and the manufacturing process) to allow the matrix, or part thereof to best placed and modified to address the medical indication and the physiological function for which it is intended. For example, the material comprising the matrix or scaffold for bladder replacement, reconstruction and/or augmentation must be sufficiently strong to support sutures without tearing, while being sufficient compliant so as to accommodate fluctuating volumes of urine.

Optimally, the matrix or scaffold should be shaped such that after its biodegradation, the resulting reconstructed bladder is collapsible when empty in a fashion similar to a natural bladder and the ureters will not be obstructed while the urinary catheter has been removed from the new organ or tissue structure without leaving a leak point. The bioengineered bladder construct can be produced as one piece or each part can be individually produced or combinations of the sections can be produced as specific parts. Each specific matrix or scaffold part may be produced to have a specific function. Otherwise specific parts may be produced for manufacturing ease. Specific parts may be constructed of specific materials and may be designed to deliver specific properties. Specific part properties may include tensile strength similar to the native tissue (e.g. ureters) of 0.5 to 1.5 MPa.sup.2 and an ultimate elongation of 30 to 100% or the tensile strength may range from 0.5 to 28 MPa.sup.2, ultimate elongations may range from 10-200% and compression strength may be <12.

A mesh-like structure formed of fibers, which may be round, scalloped, flattened, star shaped, solitary or entwined with other fibers is preferred. The use of branching fibers is based upon the same principles which nature has used to solve the problem of increasing surface area proportionate to volume increases. All multicellular organisms utilize this repeating branching structure. Branching systems represent communication networks between organs, as well as the functional units of individual organs. Seeding and implanting this configuration with cells allows implantation of large numbers of cells, each of which is exposed to the environment of the host, providing for free exchange of nutrients and waste while neovascularization is achieved. The polymeric matrix or scaffold may be made flexible or rigid, depending on the desired final form, structure and function.

In one preferred embodiment, the polymeric matrix or scaffold is formed with a polyglycolic acid with an average fiber diameter of 15 .mu.m and configured into a bladder shaped mold using 4-0 polyglactin 910 sutures. The resulting structure is coated with a liquefied copolymer, such as, for example, pol-DL-lactide-co-glycolide 50:50, 80 milligram per milliliter methylene chloride, in order to achieve adequate mechanical characteristics and to set its shape.

In a further embodiment, the scaffolds of the present invention are coated with a biocompatible and biodegradable shape-setting material. In one embodiment, the shape-setting material contains a poly-lactide-co-glycolide copolymer. In another embodiment, the shape setting material is liquefied.

In one other aspect, the scaffolds of the present invention may be treated with additives or drugs prior to implantation (before or after the polymeric matrix or scaffold is seeded with cells), e.g., to promote the regeneration of new tissue after implantation. Thus, for example, growth factors, cytokines, extracellular matrix or scaffold components, and other bioactive materials can be added to the polymeric matrix or scaffold to promote graft healing and regeneration of new tissue. Such additives will in general be selected according to the tissue or organ being reconstructed, replaced or augmented, to ensure that appropriate new tissue is formed in the engrafted organ or tissue (for examples of such additives for use in promoting bone healing, see, e.g., Kirker-Head, C. A. Vet. Surg. 24 (5): 408-19 (1995)). For example, when polymeric matrices (optionally seeded with endothelial cells) are used to augment vascular tissue, vascular endothelial growth factor (VEGF), (see, e.g., U.S. Pat. No. 5,654,273) can be employed to promote the regeneration of new vascular tissue. Growth factors and other additives (e.g., epidermal growth factor (EGF), heparin-binding epidermal-like growth factor (HBGF), fibroblast growth factor (FGF), cytokines, genes, proteins, and the like) can be added in amounts in excess of any amount of such growth factors (if any) which may be produced by the cells seeded on the polymeric matrix, if added cells are employed. Such additives are preferably provided in an amount sufficient to promote the regeneration of new tissue of a type appropriate to the tissue or organ, which is to be reconstructed, replaced or augmented (e.g., by causing or accelerating infiltration of host cells into the graft). Other useful additives include antibacterial agents such as antibiotics.

One preferred supporting matrix or scaffold is composed of crossing filaments which can allow cell survival by diffusion of nutrients across short distances once the cell support is implanted. The cell support matrix or scaffold becomes vascularized in concert with expansion of the cell mass following implantation.

The building of three-dimensional structure constructs in vitro, prior to implantation, facilitates the eventual terminal differentiation of the cells after implantation in vivo, and minimizes the risk of an inflammatory response towards the matrix, thus avoiding graft contracture and shrinkage.

The polymeric matrix or scaffold may be sterilized using any known method before use. The method used depend on the material used in the polymeric matrix. Examples of sterilization methods include steam, dry heat, radiation, gases such as ethylene oxide, gas and boiling.

The synthetic materials that make up the scaffolds may be shaped using methods such as, for example, solvent casting, compression molding, filament drawing, meshing, leaching, weaving and coating. In solvent casting, a solution of one or more polymers in an appropriate solvent, such as methylene chloride, is cast as a branching pattern relief structure. After solvent evaporation, a thin film is obtained. In compression molding, a polymer is pressed at pressures up to 30,000 pounds per square inch into an appropriate pattern. Filament drawing involves drawing from the molten polymer and meshing involves forming a mesh by compressing fibers into a felt-like material. In leaching, a solution containing two materials is spread into a shape close to the final form of the construct. Next a solvent is used to dissolve away one of the components, resulting in pore formation. (See Mikos, U.S. Pat. No. 5,514,378, hereby incorporated by reference.) In nucleation, thin films in the shape of a scaffold are exposed to radioactive fission products that create tracks of radiation damaged material. Next the polycarbonate sheets are etched with acid or base, turning the tracks of radiation-damaged material into pores. Finally, a laser may be used to shape and burn individual holes through many materials to form a structure with uniform pore sizes. Coating refers to coating or permeating a polymeric structure with a material such as, for example liquefied copolymers (poly-DL-lactide co-glycolide 50:50 80 mg/ml methylene chloride) to alter its mechanical properties. Coating may be performed in one layer, or multiple layers until the desired mechanical properties are achieved. These shaping techniques may be employed in combination, for example, a polymeric matrix or scaffold may be weaved, compression molded and glued together. Furthermore different polymeric materials shaped by different processes may be joined together to form a composite shape. The composite shape may be a laminar structure. For example, a polymeric matrix or scaffold may be attached to one or more polymeric matrixes to form a multilayer polymeric matrix or scaffold structure. The attachment may be performed by gluing with a liquid polymer or by suturing. In addition, the polymeric matrix or scaffold may be formed as a solid block and shaped by laser or other standard machining techniques to its desired final form. Laser shaping refers to the process of removing materials using a laser.

In a preferred embodiment, the scaffolds are formed from nonwoven polygycolic acid (PGA) felts and poly(lactic-co-glycolic acid) polymers (PLGA). In another preferred embodiment, the scaffold is a urinary diversion scaffold.

As described in Bertram et al. U.S. Published Application 20070276507 (incorporated herein by reference in its entirety), the polymeric matrix or scaffold of the present invention may be shaped into any number of desirable configurations to satisfy any number of overall system, geometry or space restrictions. The matrices may be three-dimensional matrices shaped to conform to the dimensions and shapes of a laminarily organized luminal organ or tissue structure. For example, in the use of the polymeric matrix for bladder reconstruction, a three-dimensional matrix may be used that has been shaped to conform to the dimensions and shapes of the whole or a part of a bladder. Naturally, the polymeric matrix may be shaped in different sizes and shapes to conform to the bladders of differently sized patients. Optionally, the polymeric matrix should be shaped such that after its biodegradation, the resulting reconstructed bladder may be collapsible when empty in a fashion similar to a natural bladder. The polymeric matrix may also be shaped in other fashions to accommodate the special needs of the patient. For example, a previously injured or disabled patient, may have a different abdominal cavity and may require a bladder replacement scaffold, a bladder augmentation scaffold, a bladder conduit scaffold, and a detrusor muscle equivalent scaffold adapted to fit.

In one aspect, the present invention contemplates additional scaffolds suitable for use with the smooth muscle cell populations described herein. For example, scaffolds suitable for implantation into the lung may be provided.

A. Augmentation or Replacement Scaffolds

In one other aspect, the polymeric matrix or scaffold is shaped to conform to part of a bladder. In one embodiment, the shaped matrix is conformed to replace at least about 50%, at least about 60%, at least about 70%, at least about 80%, at least about 90%, or at least about 95% of the existing bladder of a recipient. In one other aspect, the polymeric matrix or scaffold is shaped to conform to 100% or all of a bladder.

In one embodiment, the polymeric matrix comprises a first implantable, biocompatible, synthetic or natural polymeric matrix or scaffold having at least two separate surfaces, and a second implantable, biocompatible, synthetic or natural polymeric matrix or scaffold having at least two separate surfaces, which are adapted to mate to each other and shaped to conform to at least a part of the luminal organ or tissue structure in need of the treatment when mated. The first and second polymeric matrices may be formed from one integral unit subdivided into two or more distinct parts, or from two or more distinct parts, adapted to mate. In some embodiments, the first and second polymeric matrices once mated may be used for reconstruction, augmentation, or replacement of a luminal organ or tissue structure.

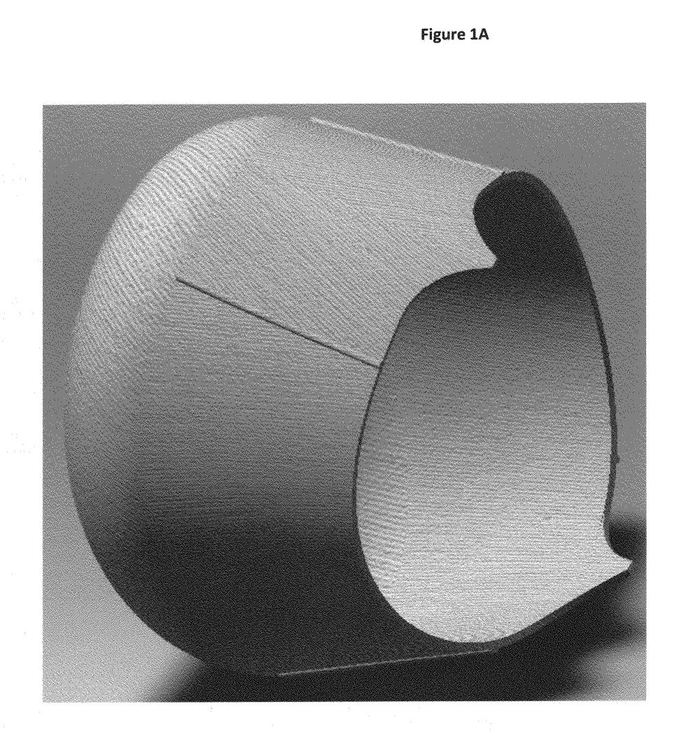

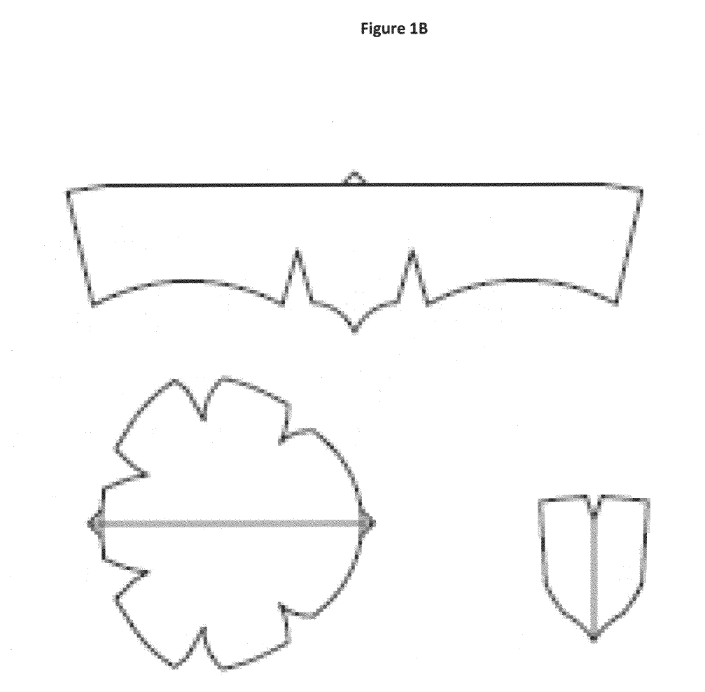

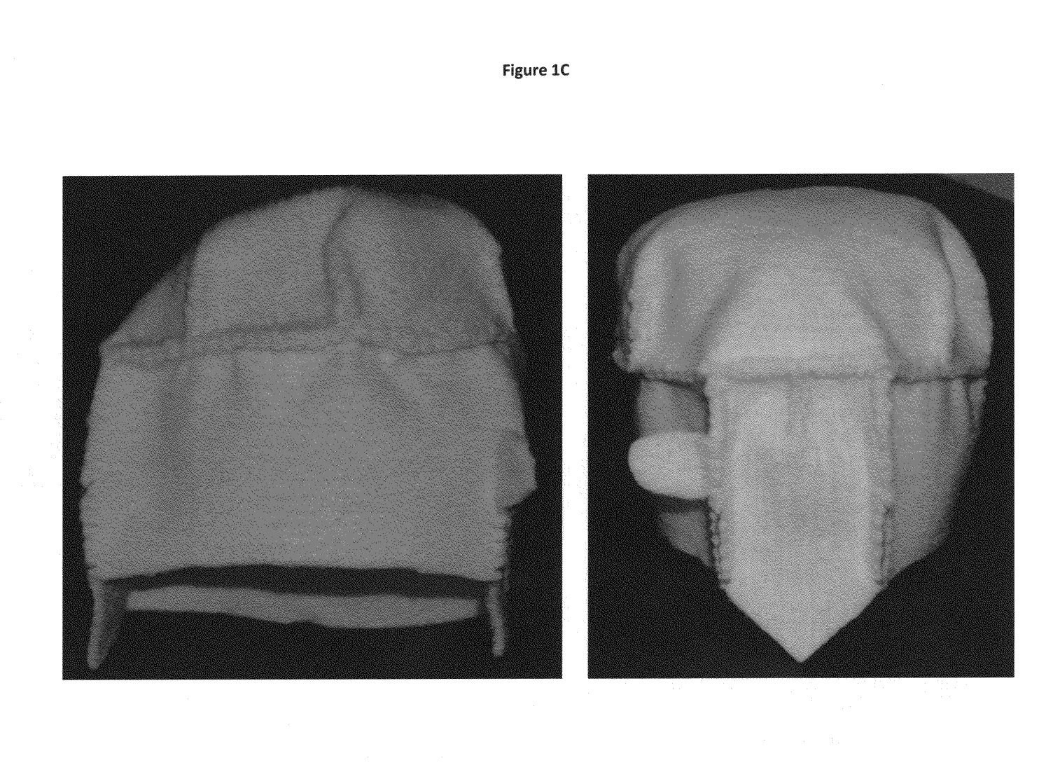

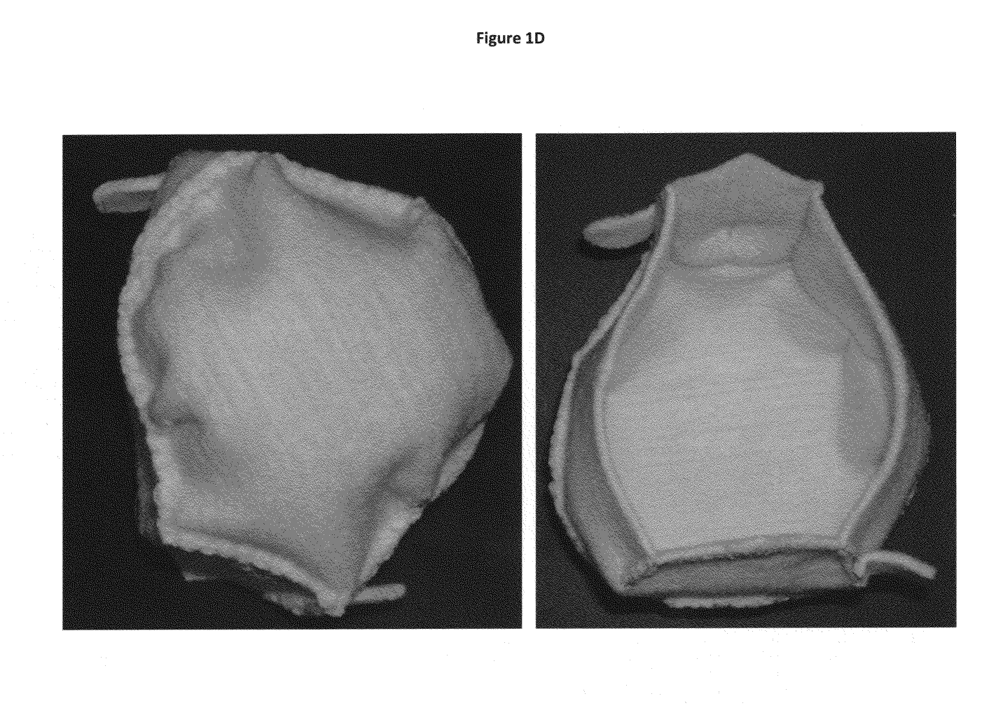

In some embodiments, the first and second polymeric matrices are symmetrical, while in other embodiments, the first and second polymeric matrices are asymmetrical. In one embodiment, the first polymeric matrix or scaffold has a hemispherical or quasi-hemispherical shape having a closed, domed end and an open, equatorial border, and the second polymeric matrix or scaffold is a collar adapted to mate with the equatorial border of the first polymeric matrix. In another embodiment, the first and second polymeric matrices are each hemispherical or quasi-hemispherical in shape, having a closed, domed end and an open, equatorial border. In yet another embodiment, the first and second polymeric matrices each comprise a circular or semi-circular base and at least 2 petals radially extending from each base. In this embodiment, the bases and petal shaped portions of the first and the second polymeric matrices are mated to create a hollow spherical or quasi-spherical matrix or scaffold such that a flanged longitudinal, elliptical opening is created on one side of the mated polymeric matrices, and a circular opening is created on the side opposite the longitudinal opening. In another embodiment, the first and second polymeric matrices are made from 3 parts comprising a top, a front and a sidepiece, adapted to mate. In this embodiment, the 3 distinct parts are mated using at least 3, preferably four vertical seams, thereby forming a crown shaped neo-bladder construct. The crown shaped constructs are preferably used alone as a device for luminal organ reconstruction, augmentation, or replacement. In one embodiment, the construct is a bladder augmentation scaffold. One example of a bladder augmentation scaffold is depicted in FIG. 1A-D.

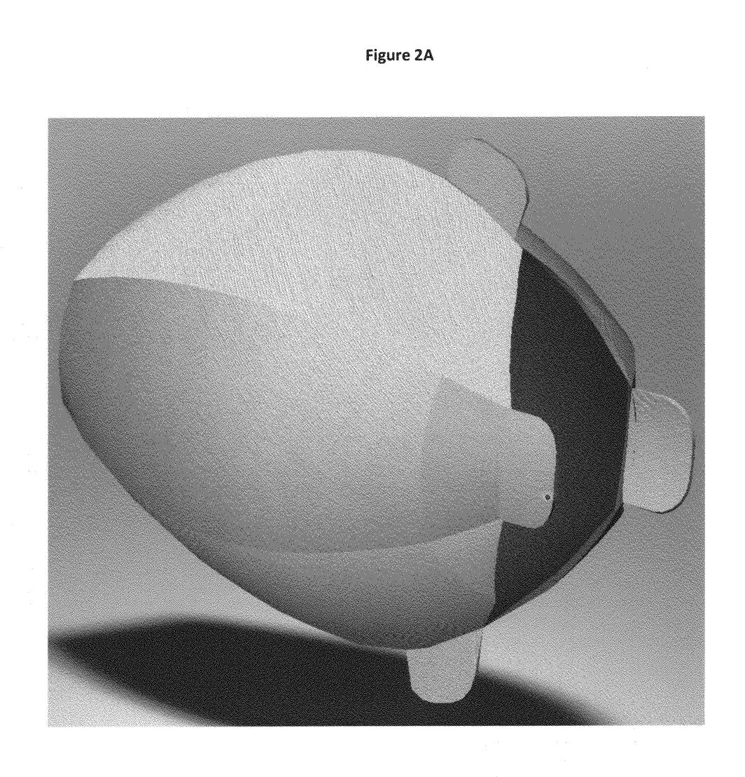

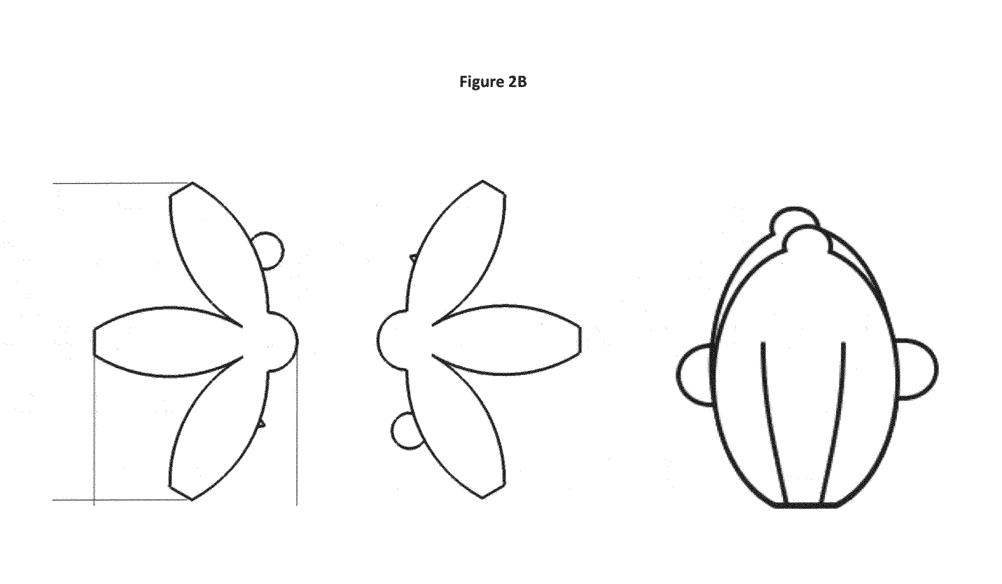

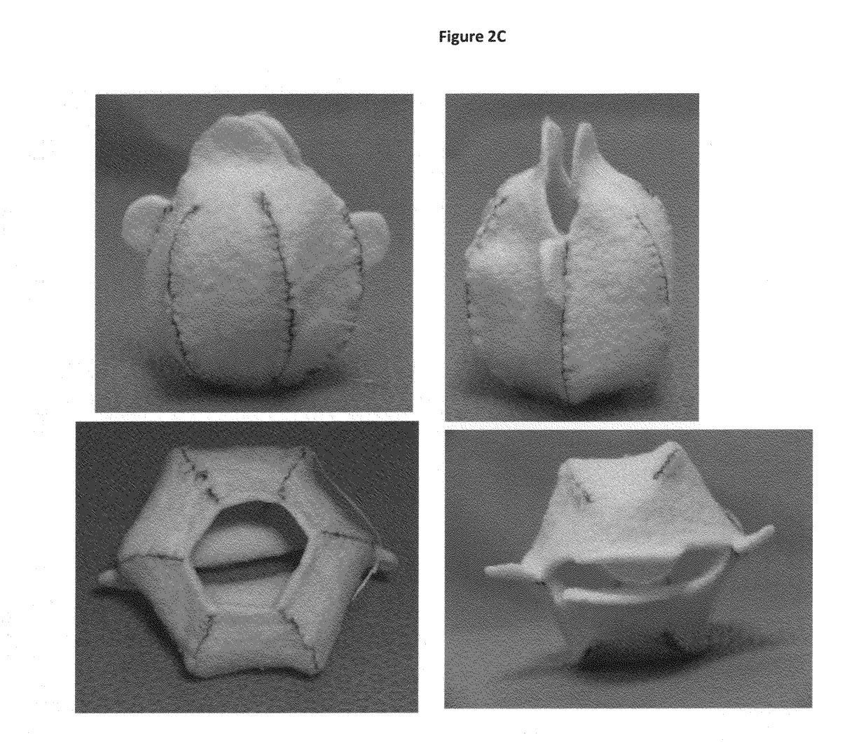

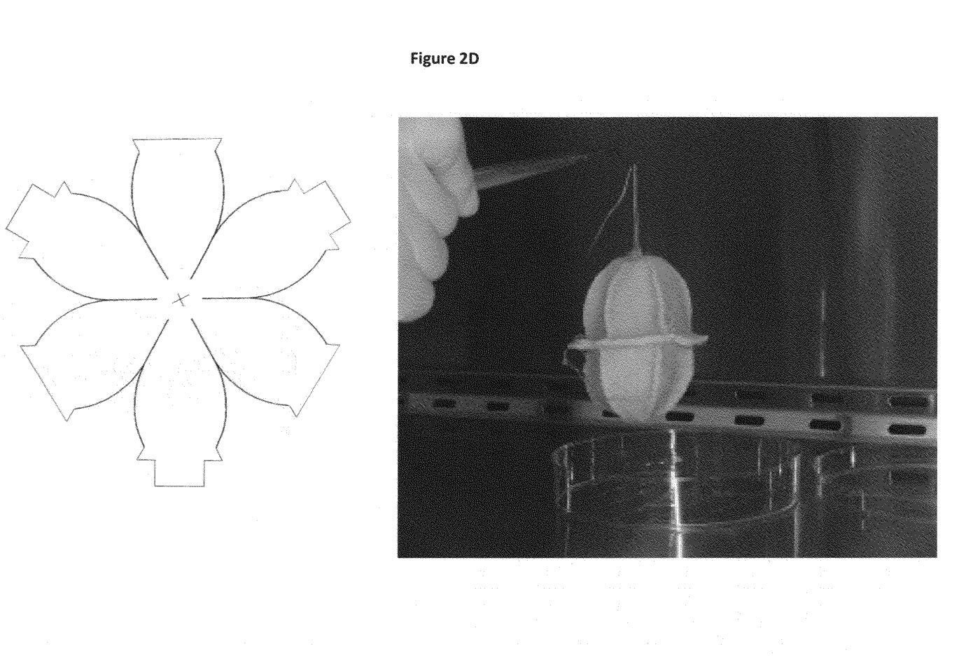

In another embodiment, the construct is a bladder replacement scaffold. One example of a bladder replacement scaffold is depicted in FIG. 2-D.

Additionally, the first polymeric matrix, the second polymeric matrix, or both, may contain at least one receptacle or port adapted to receive a tubular vessel or insert where the connection of the construct to a native vessel or tube is necessary. The vessels or inserts are themselves, for example, cylindrical or tubular shaped polymer matrices, each having at least one flange located at a first end of the cylindrical polymer. The vessels or inserts are, preferably, composed of the same biocompatible material as the first or second polymeric matrices described above. In some embodiments, the vessel or insert also contains a washer adapted to fit around the cylindrical or tubular vessel or insert polymer matrix. For example, the washer is a hydrogel. The cylindrical or tubular vessel or insert may optionally contain a washer. The washer may be hydrogel. Additionally, the cylindrical or tubular insert may be self-stabilizing.

In another embodiment, the receptacles or ports adapted to receive tubular vessels or inserts where the connection of the scaffold or matrix (once seeded with cells) to a native vessel or tube is necessary also applies to other the matrices discussed below.

In one aspect, the scaffold is an organ or tissue structure replacement scaffold that includes at least two matrices. In one embodiment, the scaffold comprises a first matrix having a first surface and a second matrix having a first surface. The first matrix and the second matrix may be configured or adapted to mate. In another embodiment, the first matrix and the second matrix may be shaped to conform to at least a part of a luminal organ when mated. The first and second matrix may comprise a biocompatible material. The biocompatible material may comprise a biodegradable material.

In one embodiment, the first matrix may have a hemispherical shape with a closed end and an open, equatorial border, and the second matrix may have a collar configured or adapted to mate with the equatorial border of the first matrix. The closed end may be domed. In another embodiment, the first matrix and the second matrix may each have a hemispherical shape having a closed end and an open, equatorial border. The closed end may be domed. In yet another embodiment, the first matrix may further comprises a flanged region along at least one border of the first matrix. The second matrix further may comprise a flanged region along at least one border of the second matrix, and wherein the flanged region of the second matrix is adapted to mate with the flanged region of the first matrix.

In one embodiment, the scaffold comprises a first, biocompatible matrix and a second, biocompatible matrix, where the first and second matrix may each comprise a base and may be configured or adapted to mate. In one embodiment, the first and second matrices may be shaped to conform to at least a part of a luminal organ when mated. In another embodiment, the first and second matrix may further comprise at least two petals radially extending from each base.

In one other embodiment, each of the first and second matrices may be originally derived from a template comprising a base and at least four petals. In one configuration, a pair of opposing petals may be shorter in length than the other petals. In another embodiment, the first and second matrices may be two distinct units adapted to mate.

In one embodiment, the bases of the first and second matrixes are adapted to mate. In some embodiments, the first and second matrices are mated via the petal shaped portions of the first and second matrixes.

In other embodiments, the first and second matrices may be configured or adapted to form a hollow spherical or quasi-spherical shape with a longitudinal opening at a first mating point between the first and second matrices and a circular opening at a second mating point between the first and second matrices that is opposite the longitudinal opening. The scaffold may further include at least one flap incorporated into the base of the first or second matrix. In another embodiment, the longitudinal opening has a lip and at least one flap is disposed at the lip of the longitudinal opening.

In another aspect, the matrix or matrices may be connectable to a native vessel. In one embodiment, the first matrix, the second matrix, or both, are each configured or adapted to receive a native vessel. In another embodiment, the first matrix, the second matrix, or both, further comprise at least one receptacle. The at least one receptacle may be configured or adapted to receive a tubular insert. The tubular insert may be disposed within the receptacle. In some embodiments, the tubular insert has an end. The insert may have at least one flange located at this end. In another embodiment, the tubular insert may be configured or adapted to connect to a native vessel. In a further embodiment, the scaffold has a surface and a washer disposed around the tubular insert. The washer may be configured or adapted to form a watertight seal between the flange and the surface of the construct. In some embodiments, the washer comprises a hydrogel.

FIGS. 1 and 2 provide representative depictions of scaffold configurations that include at least two matrices.

In one aspect, the scaffold is an organ or tissue structure augmentation scaffold that includes one or more matrices. In one embodiment, the scaffold includes a first matrix having a base and a plurality of notches, wherein the first matrix is adapted to form a hemi-shape that conforms to at least a part of a luminal organ when assembled. In another embodiment, the scaffold includes a second and a third matrix, wherein the first, second and third matrices may be configured or adapted to mate and are shaped to conform to at least part of the luminal organ when mated. The first, second and third polymeric matrices may be derived from a template comprising three subdivided parts. In another embodiment, the first, second and third matrices are derived from three distinct templates and may are configured or adapted to mate. In one other embodiment, the first, second, and/or third matrix comprise a biocompatible material. The biocompatible material may comprise a biodegradable material.

In one other aspect, the scaffold is made up of parts having different shapes or configurations. In one embodiment, the scaffold may include a first, second and third polymeric matrices that are correspond to a top piece, a front piece, and a side piece, respectively, that when mated together form a first crown shape. In another embodiment, the front piece and the side piece may each comprise a first edge and a second edge. The first edge of the front piece may be joined to the first edge of the side piece. The second edge of the front piece may be joined to the second edge of the side piece. In one other embodiment, the first edges may be joined by a seam and/or the second edges may be joined by a seam. In other embodiments, the front piece may include a notch having a first edge and a second edge. The first and second edges may be joined, such as, for example by a seam. In another embodiment, the top piece may have a first edge, the side piece has a third edge, and the front piece has a third edge. The first first edge of the top piece may be joined to the third edge of the side piece and/or the first edge of the top piece may be joined to the third edge of the front piece. The first and third edges may be joined by a seam. In another embodiment, each notch may have a first edge and a second edge. These edges may be joined, such as, for example by a seam. In other embodiments, the side piece may include at least one flap.

In all embodiments, each individual matrix or all matrices in a scaffold may comprise a biodegradable material. The material may be selected from the group consisting of polyglycolic acid, polylactic acid and a copolymer of glycolic acid and lactic acid. In other embodiments, the matrix or matrices comprise polyglycolic acid and a copolymer of glycolic acid and lactic acid.

In one embodiment, the luminal organ is a tubular or hollow organ. The organ may be a genitourinary organ. In another embodiment, the genitourinary organ is selected from the group consisting of bladder, ureters and urethra. In one other embodiment, the genitourinary organ is a bladder or a bladder segment. In some embodiments, the scaffolds used are configured or adapted to form regenerated bladder tissue in vivo that exhibits the compliance of natural bladder tissue.

In one embodiment, the mated matrices with deposited cells form an implantable construct. In another embodiment, the at least first cell population comprises a muscle cell population as described herein. The muscle population may be a smooth muscle cell population.

In another embodiment, the scaffold may have at least a first cell population deposited on or in a first surface of the first matrix, a first surface of the second matrix, or both. In one other embodiment, the scaffold may further include a second population of cells deposited on or in a second surface of the first matrix, a second surface of the second matrix, or both. The second population of cells comprises urothelial cells.

The augmentation and replacement scaffolds described herein, as well as methods of making and using the same, are further described in Bertram et al. U.S. Published Patent Application No. 20070276507 (incorporated herein by reference in its entirety).

B. Urinary Conduit Scaffolds

The present invention provides neo-urinary diversion or conduit scaffolds that can be seeded with cells and used as a replacement for gastrointestinal tissue in the construction of a urinary diversion in a subject. For example, the neo-urinary diversions described herein may have application after radical cystectomy for the treatment of patients who would otherwise undergo an ileal loop diversion.

In one aspect, the present invention contemplates conduit scaffolds or matrices suitable for use as urinary diversions in a subject in need formed from the methods described herein. One end of the conduit scaffold may be connected to one or more ureters and the other end may be connected to a urine reservoir that is external to the subject's body. In one embodiment, the conduit may exit the subject's body via a stoma. In another embodiment, the polymeric matrix comprises a first implantable, biocompatible, synthetic polymeric matrix or scaffold provided in a tubular form. In some embodiments, the tubular scaffold comprises a first end configured to connect to a ureter of the subject. In another embodiment, the first scaffold further includes a second end configured to form a stoma or sphincter in the subject. In another embodiment, the first scaffold further includes at least one side opening configured to connect to a least one ureter. In some embodiments, the first scaffold includes a first side opening configured to attach to a first ureter and a second side opening configured to attach to a second ureter.

In one aspect, the scaffold is designed to be flexible as to the attachment of one or both ureters in the subject. In one embodiment, the scaffold may have one or more openings for attachment of a ureter on the side of the tubular structure. In another embodiment, the scaffold may have an opening at one end of the tubular structure for attachment of a ureter. The attachment of a ureter to one end of the structure rather than the side may present less strain on the ureter if the distance between the end of the ureter to be attached and the scaffold end is less than the distance between the end of the ureter and the side of the scaffold.

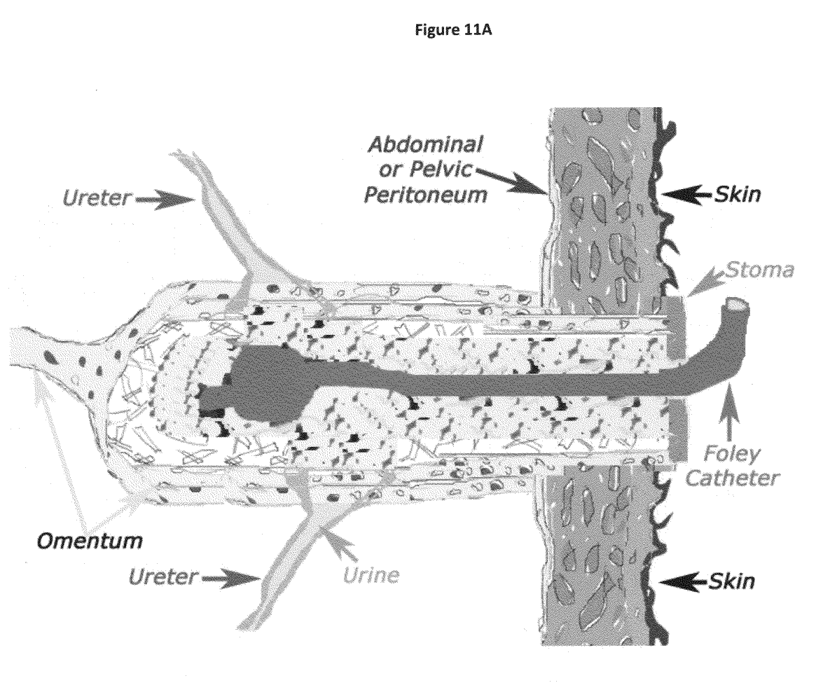

In one aspect, the tubular conduit scaffold comprises one end of the tube that serves as the outflow end for urine that passes from one or both ureters through the tubular scaffold and ultimately out of the recipient. In one embodiment, the outflow end of the scaffold is configured to terminate at the wall of the abdominal cavity of the recipient. FIG. 11B (panel A) illustrates an exemplary configuration for the scaffold.

In another embodiment, the outflow end of the scaffold is configured to extend through the abdominal wall, i.e., transabdominal, and connect directly to the subcutaenous layer of the skin stoma, i.e., percutaneous. FIG. 11B (panel B) illustrates an exemplary configuration for the scaffold.

Those of ordinary skill in the art will appreciate that the different configurations will depend upon the particular dimensions of the abdominal cavity of the recipient.

In one other embodiment, the tubular structure comprises a first end comprising an even edge and a second end comprising a non-uniform or uneven edge. The non-uniform edge may include a circular base with a number of petals radially extending from the base. The number of petals may be 1, 2, 3, 4, 5, or 6. The uneven edge may comprise a series of petals such as, for example, those shown in FIG. 3. In one embodiment, the tubular structure has a form suitable for use as a urinary diversion system or a conduit in a patient in need. In another embodiment, the system diverts urine from one or more ureters to an abdominal wall section such as, for example, in the case of a ureterostomy. In other embodiments, the system diverts urine from the bladder to an abdominal wall section such as, for example, in the case of a cystostomy. In one other embodiment, the system connects the bladder to the urethra. In yet another embodiment, a first system may divert urine from one or more ureters to an abdominal wall section and a second system may divert urine from the bladder to an abdominal wall section. In all embodiments, the system may divert urine from one or more ureters to an abdominal wall section such as, for example, in the formation of a stoma.

In another embodiment, the tubular matrix or scaffold is a urinary diversion or conduit scaffold.

In one embodiment, the tubular structure of the urinary diversion system is of rectangular, circular, or triangular cross sectional area. FIG. 3A illustrates some of the different cross sectional configurations contemplated herein.

In another embodiment, tubular structure retains sufficient rigidity to remain patent following implantation. In one other embodiment, the tubular structure's rigidity is retained with or without the use of a catheter in its lumen. Where a catheter is used, it can be placed into the luminal space of the tubular structure to provide additional patency.

In one other embodiment, the conduit scaffold may further include a second scaffold in the form of a round or ovoid connector configured to connect the first end of the first scaffold to a ureter. In yet another embodiment, the conduit scaffold may further include a third scaffold in the form of a washer-ring configured to form a stoma or sphincter with the second end of the first tubular scaffold to create a stoma in a subject. FIG. 3B illustrates variations of a urinary diversion construct (A—open claim ovoid; B—open claim ovoid receptacle; C—closed ovoid receptacle and three tubes).

In some embodiments, the tubular structure may include a washer structure for connection to a tissue, organ or body part to achieve anastomosis for the creation of a continent stoma or sphincter. In another embodiment, the washer is provided with a thickness of about less than 1 mm, about less than 1.5 mm, about less than 2 mm, about less than 2.5 mm, about less than 3 mm, about less than 3.5 mm, about less than 4 mm, about less than 4.5 mm, or about less than 5 mm.

In one embodiment, the urinary diversion or conduit scaffold is shaped into the configuration shown in FIG. 3.

In one other embodiment, the tubular structure comprises a first end comprising an even edge and a second end comprising a non-uniform or uneven edge. The non-uniform edge may include one or more fasteners configured for attachment to an external region of the subject, such as in the formation of a stoma external to the subject. In one embodiment, the first and second ends of the tubular structure may be in the form illustrated in FIG. 3. The number of fasteners may be 1, 2, 3, 4, 5, or 6.

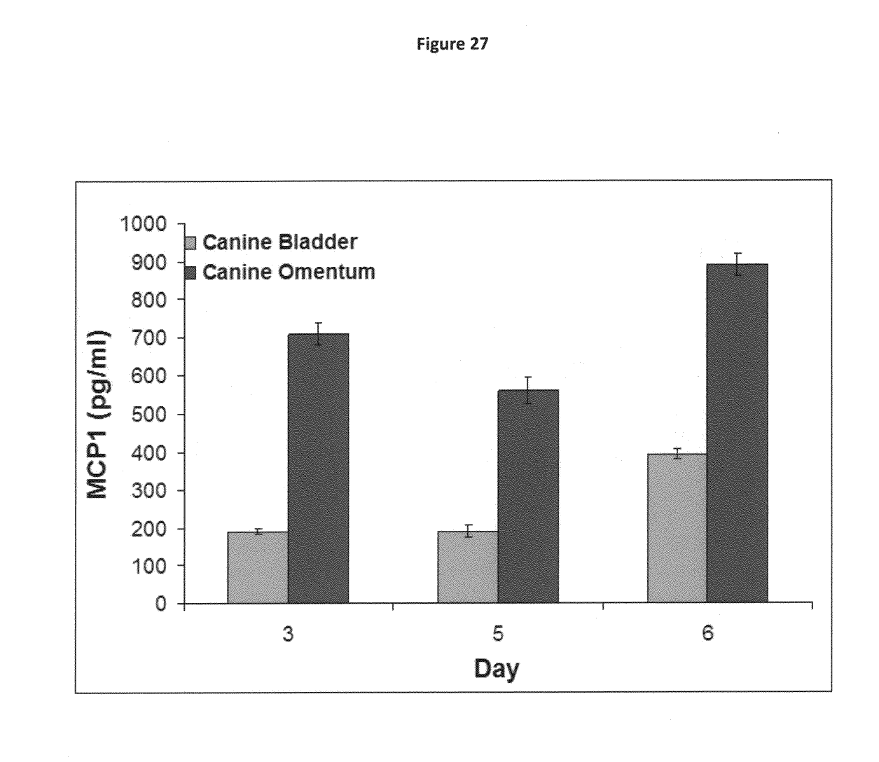

In another embodiment, the tubular scaffold is in the form depicted in FIG. 27.

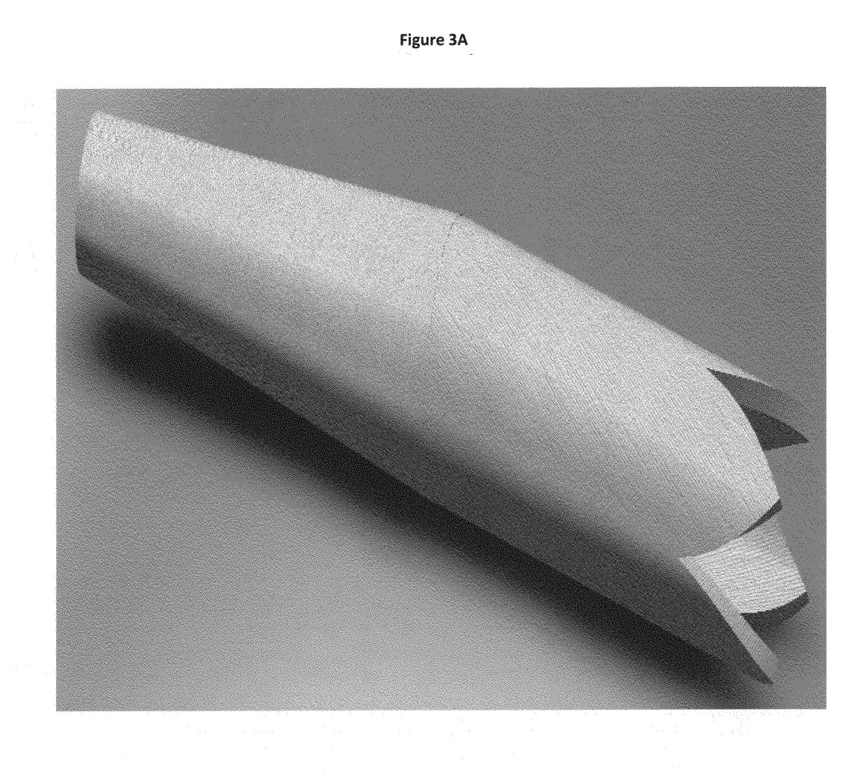

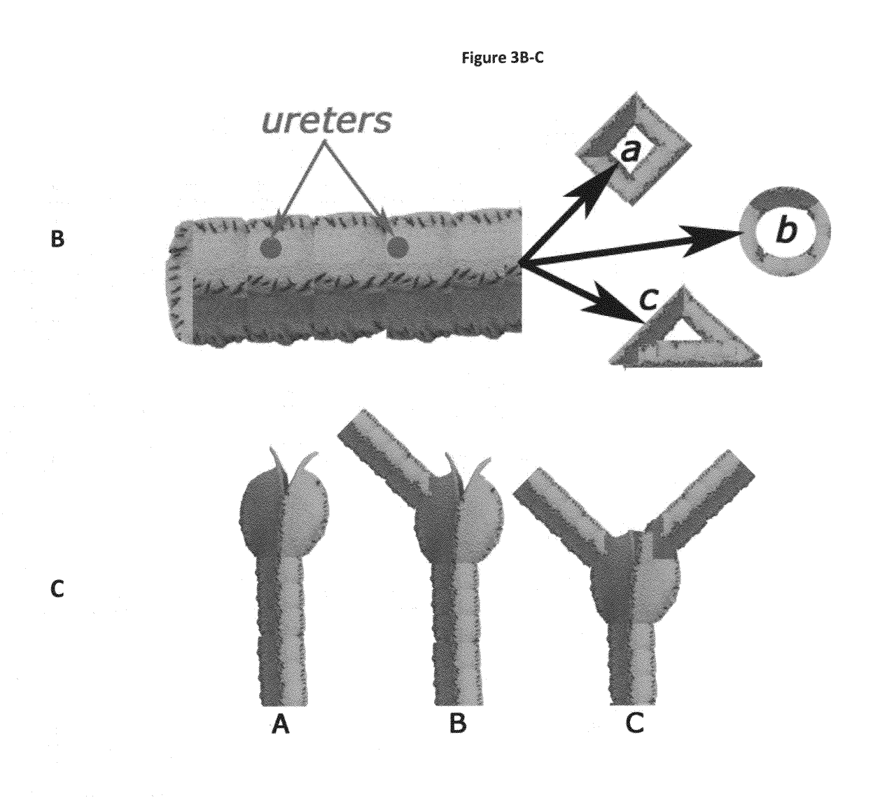

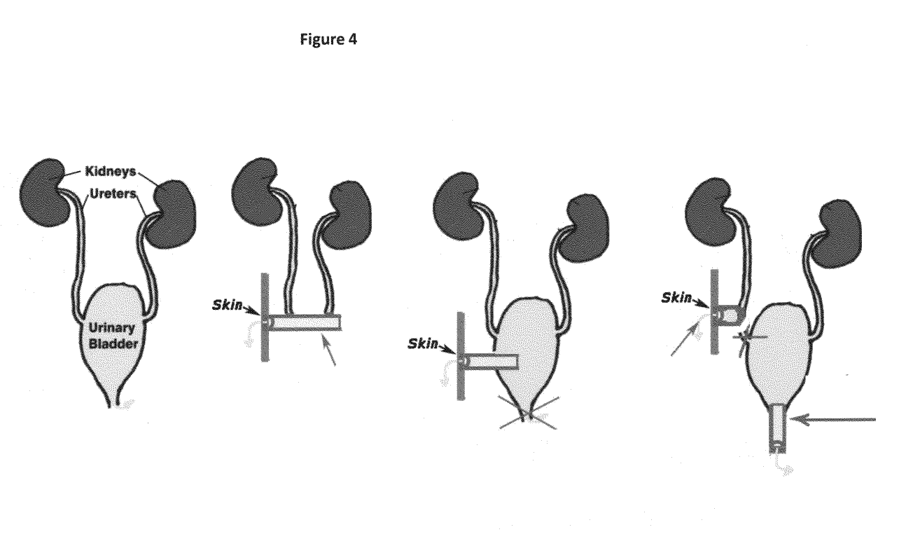

FIG. 4A depicts a part of the normal anatomy for the human urinary system.

In one embodiment, the tubular structure has a form suitable for use as a urinary diversion or a conduit in a patient in need. In another embodiment, the conduit diverts urine from one or more ureters to an abdominal wall section such as, for example, in the case of a ureterostomy (FIG. 4D). In other embodiments, the conduit diverts urine from the bladder to an abdominal wall section such as, for example, in the case of a cystostomy (FIG. 4B). In one other embodiment, the conduit connects the bladder to the urethra (FIG. 4D). In yet another embodiment, a first conduit may divert urine from one or more ureters to an abdominal wall section and a second conduit may divert urine from the bladder to an abdominal wall section. In all embodiments, the conduit may divert urine from one or more ureters to an abdominal wall section (FIG. 4B). In all embodiments, the conduit may be configured to form a stoma.

In one embodiment, the tubular structure of the urinary diversion or conduit scaffold is of rectangular, circular, or triangular cross sectional area. In another embodiment, the tubular structure retains sufficient rigidity to remain patent following implantation. In one other embodiment, the tubular structure's rigidity is retained with or without the use of a catheter in its lumen. In some embodiments, a urinary diversion scaffolds further include a catheter configured to be placed in the luminal space of tubular structure upon implantation. In one embodiment, the catheter is a Foley-like balloon catheter. Where a catheter is used, it can be placed into the luminal space of the tubular structure to provide additional patency. Those of ordinary skill in the art will appreciate that other catheters known in the art may be suitable for use with the present invention.

In another embodiment, the thickness of the tubular wall of the scaffolds will be less than about 2 mm, less than about 2.5 mm, less than about 3.5 mm, less than about 4 mm, less than about 4.5 mm, less than about 5 mm, less than about 5.5 mm, or less than about 6 mm.

In some embodiments, the scaffolds may have variable outer and inner diameters. In one embodiment, the ends of the scaffold may be flared, non-flared, sealed, or rounded.

In other embodiments, the scaffold is permeable to urine. In one embodiment, the scaffold's pore size is about greater than about 0 microns to about 500 microns. In another embodiment, the pore size is from about 100 microns to about 200 microns. In another embodiment, the pore size is from about 150 microns to about 200 microns. In other embodiments, the pore size is about 100 microns, about 110 microns, about 120 microns, about 130 microns, about 140 microns, about 150 microns, about 160 microns, about 170 microns, about 180 microns, about 190 microns, or about 200 microns. In some embodiments, the pore size is about 100 microns, about 200 microns, about 300 microns, about 400 microns, about 500 microns, or about 600 microns. In other embodiments, the scaffold includes a pore architecture that is a single pore size distribution, multiple pore size distribution, or a pore gradient distribution.

In another embodiment, the scaffold material is suturable and may form connections with tissue that are resistant to leakage.

In other embodiments, the tubular scaffold material is selected to maintain patency throughout the duration of implantation use, support cell attachment and the in-growth of host tissue, and retain flexibility. In another embodiment, the material will have a burst strength that exceeds the pressures to which it will be exposed during normal in vivo fluid cycling. In other embodiments, the material will have a degradation time commensurate with host tissue in-growth.

The conduit scaffolds described herein, as well as methods of making and using the same, are further described in Ludlow et al. U.S. Published Patent Application No. 20100131075 (incorporated herein by reference in its entirety).

C. Muscle Equivalents

In one aspect, the polymeric matrix or scaffold of the present invention is a muscle equivalent scaffold. In one embodiment, the muscle equivalent scaffold is a detrusor muscle equivalent scaffold. In another embodiment, the scaffold is suitable for laparoscopic implantation.

In one aspect, the polymeric matrix comprises a polymeric matrix or scaffold shaped to conform to at least a part of the organ or tissue structure in need of said treatment and of a sufficient size to be laparoscopically implanted. In certain embodiments, the polymeric matrix or scaffold of the invention is between about 3 and about 20 cm in length. In one embodiment the polymeric matrix or scaffold is about 20 cm in maximal length. In another embodiment, the polymeric matrix or scaffold is about 15 cm in maximal length. In another embodiment, the polymeric matrix or scaffold is about 10 cm in maximal length. In another embodiment, the polymeric matrix or scaffold is about 8 cm in maximal length. In another embodiment, the polymeric matrix or scaffold is about 4 cm in maximal length. In yet another embodiment, the polymeric matrix or scaffold is about 3 cm in maximal length. In certain embodiments, the polymeric matrix or scaffold of the invention is between about 1 and about 8 cm in width. In some embodiments, the polymeric matrix or scaffold is about 4 cm in maximal width. In other embodiments, the polymeric matrix or scaffold is about 3 cm in maximal width. In yet other embodiments, the polymeric matrix or scaffold is about 5 cm in maximal width.

In one embodiment, the polymeric matrix or scaffold has a three-dimensional (3-D) shape. In another embodiment, the polymeric matrix or scaffold has a flat shape. In one embodiment, the flat-shaped polymeric matrix or scaffold comprises pre-treated areas to allow more flexibility. In certain embodiments, the pre-treated areas are coated in the areas to be creased. In one embodiment, the polymeric matrix or scaffold is sufficiently malleable to be rolled, folded, or otherwise shaped for implantation through a laparoscope tube and/or port. In such embodiments, the polymeric matrix or scaffold is sufficiently malleable to be unrolled, unfolded, or otherwise returned to shape following insertion through the laparoscope tube and/or port. In one embodiment, the polymeric matrix or scaffold is cut into 2, 3, 4, 5, 6, 7, 8, 9 or 10 strips prior to implantation through a laparoscope tube and/or port. In certain embodiments, the 2, 3, 4, 5, 6, 7, 8, 9 or 10 strips are mated prior to implantation through a laparoscope tube and/or port. The 2, 3, 4, 5, 6, 7, 8, 9 or 10 strips may be mated using glue, staples, sutures, or other technique known to one of ordinary skill in the art. In such embodiments the 2, 3, 4, 5, 6, 7, 8, 9 or 10 mated strips are folded and/or stacked to pass through a laparoscope tube and/or port. In such embodiments, the 2, 3, 4, 5, 6, 7, 8, 9 or 10 strips are unfolded and/or unstacked following insertion through the laparoscope tube and/or port. In some embodiments, the previously placed mating means are tightened as appropriate following insertion through the laparoscope tube and/or port.

In one embodiment, the polymeric matrix comprises a first implantable, biocompatible, synthetic or natural polymeric matrix or scaffold provided in the form of a patch or in the form of a strip. In one embodiment, the patch has a form suitable for use as a detrusor muscle equivalent in the bladder of a patient in need. In one other embodiment, the patch has a form suitable for increasing the volume capacity of the existing bladder of a patient in need. In certain embodiments, the patch increases the bladder size between about 50 mL and about 500 mL. In some embodiments, the patch would increase bladder size in increments of 50 mL. In some embodiments, the patch increases the bladder size about 450 mL. In one embodiment, a surface area increase of 30 cm² increases the volume of a 200 mL bladder to 250 mL. In another embodiment, an increase of 25 cm² increases the volume of a 350 mL bladder to 400 mL. In one embodiment, the scaffold has a two-dimensional surface area of about 30 cm². In another embodiment, the scaffold has a two-dimensional surface area of about 25 cm². In one embodiment, the patch is in the form of a strip, disc, square, ellipsoid, or any other appropriate configuration. In other embodiments, the patch is provide in a pre-folded form, e.g., like an accordion.

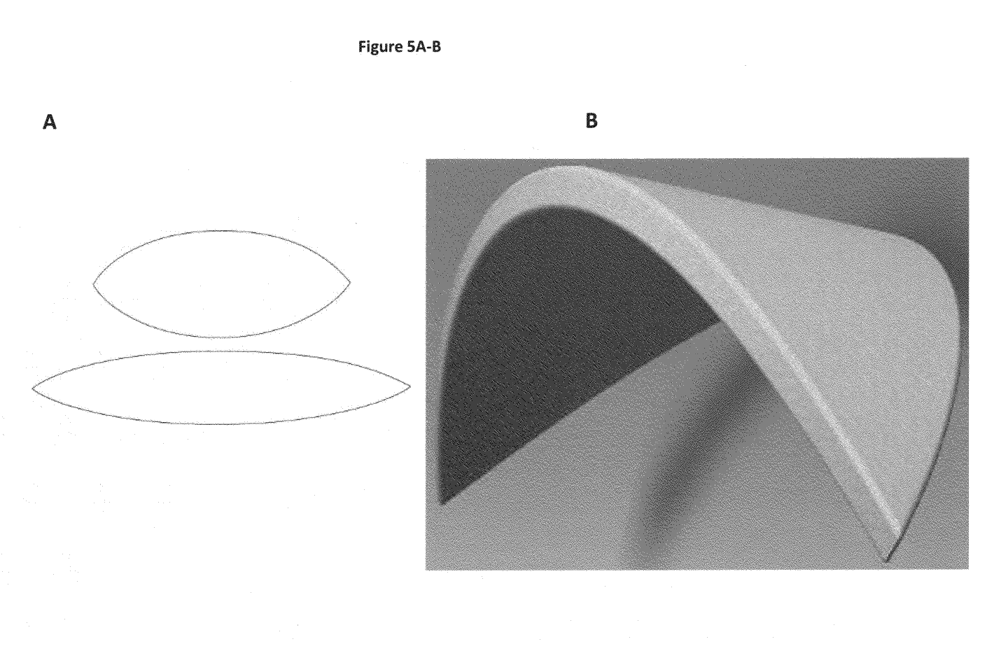

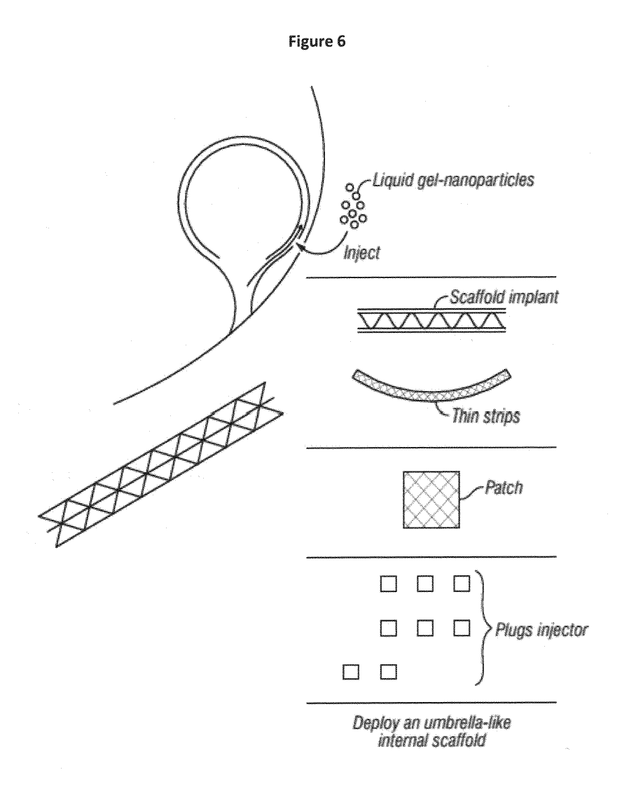

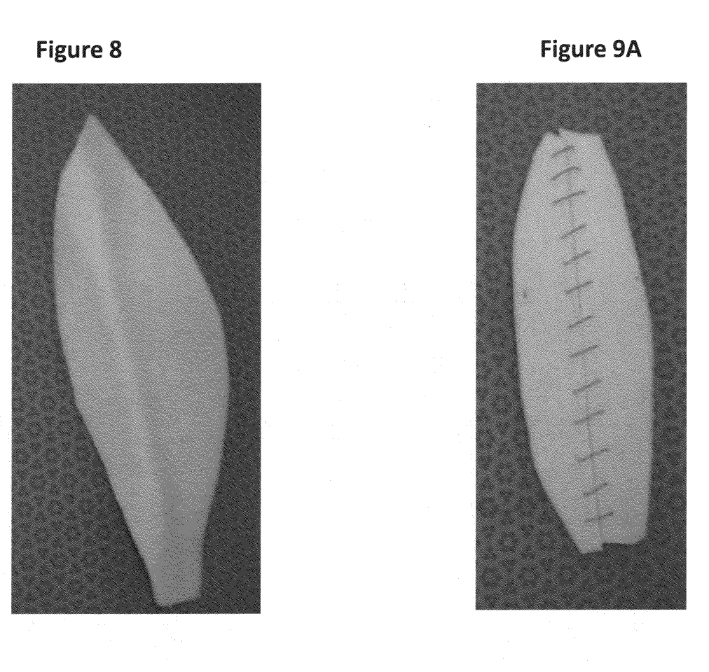

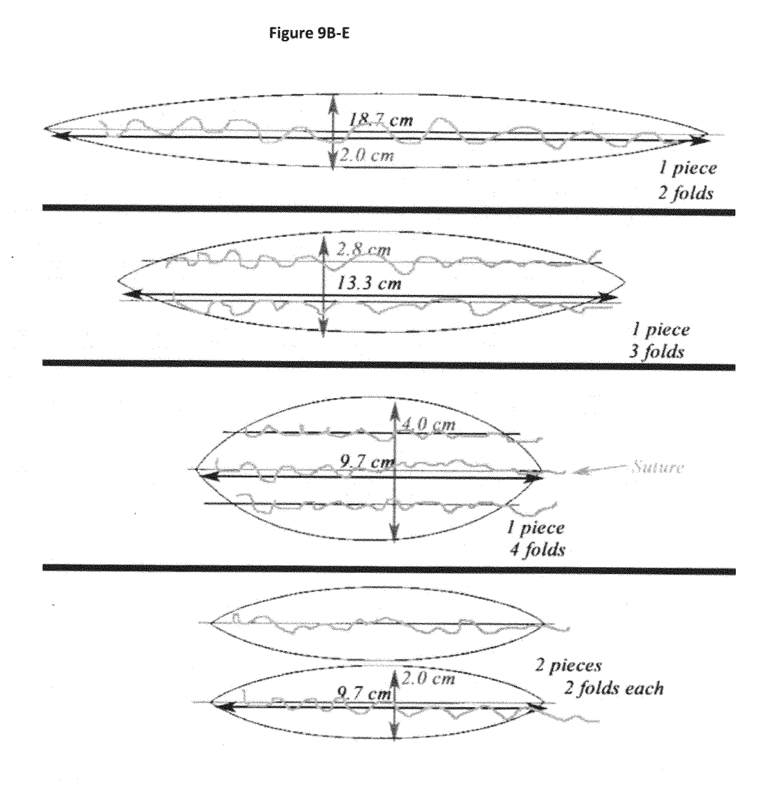

FIG. 5A-B show examples of a muscle equivalent scaffold or polymeric matrix. In one embodiment, the polymeric matrix or scaffold is in the shape of a double wedge, e.g., the shape shown in FIG. 5A. In another embodiment, the polymeric matrix is shaped into one of the configurations shown in FIGS. 6-9. In FIG. 9D, the folds allow the implant to pass through a 12 mm tube.

In all embodiments, the polymeric matrix or scaffold is shaped so as to minimize the strain on both the bladder and matrix or scaffold.

In another embodiment, the polymeric matrix comprises a first implantable, biocompatible, synthetic or natural polymeric matrix or scaffold provided in the form of a patch or in the form of a strip. In one embodiment, the patch has a form suitable for use as a detrusor muscle equivalent in the bladder of a patient in need. In one other embodiment, the patch has a form suitable for increasing the volume capacity of the existing bladder of a patient in need. In some embodiments, the patch would increase bladder size in increments of 50 mL. In one embodiment, the patch is in the form of a strip, disc, square, ellipsoid, or any other appropriate configuration. In other embodiments, the patch is provide in a pre-folded form, e.g., like an accordion.

In one embodiment, the polymeric matrix is shaped into one of the configurations shown in FIGS. 1-9.

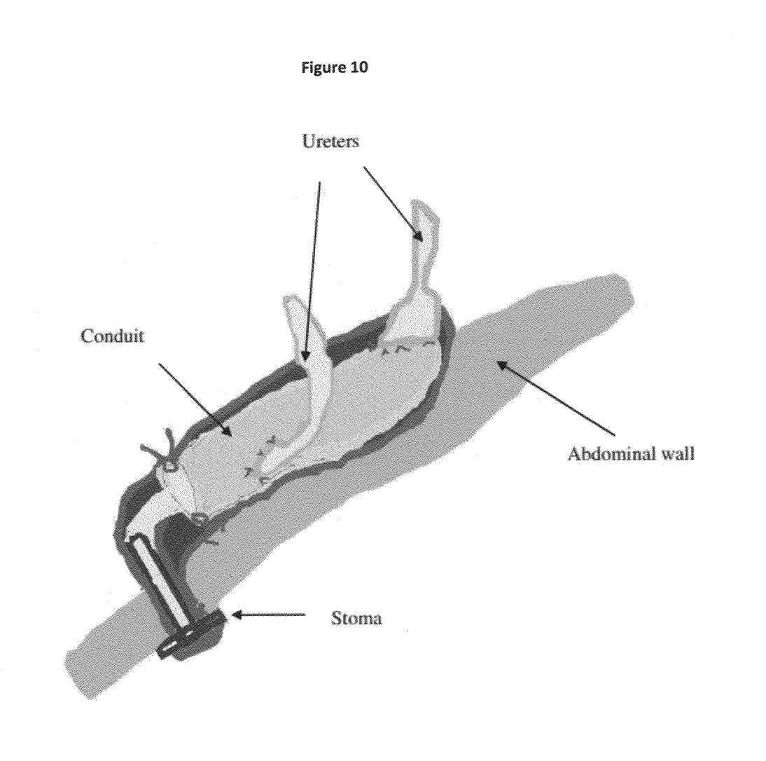

In another embodiment, the polymeric matrix is implanted into a subject in need according to one of the configurations shown in FIGS. 10-13.

In all embodiments, the biocompatible material used for these matrices or scaffolds is, for example, biodegradable. In all the embodiments, the biocompatible material may be polyglycolic acid. In all embodiments, the polymeric matrix or scaffold is coated with a biocompatible and biodegradable shaped setting material. In one embodiment, the shape setting material may comprise a liquid copolymer. In another embodiment, the liquid co-polymer may comprise a liquefied lactide/glycolide copolymer. In one embodiment, the liquid co-polymer may comprise poly-DL-lactide-co-glycolide.

The muscle equivalent scaffolds described herein, as well as methods of making and using the same, are further described in Ludlow et al. U.S. Published Patent Application No. 20100131075 (incorporated herein by reference in its entirety).

5. Constructs

In one aspect, the invention provides one or more polymeric scaffolds or matrices that are seeded with at least one cell population. Such scaffolds that have been seeded with a cell population and may be referred to herein as “constructs”. In one embodiment, the cell-seeded polymeric matrix or matrices form a neo-bladder construct selected from the group consisting of a bladder replacement construct, a bladder augmentation construct, a bladder conduit construct, and a detrusor muscle equivalent construct.

Those of skill in the art will appreciate that the seeding or deposition of one or more cell populations described herein may be achieved by various methods known in the art. For example, bioreactor incubation and culturing, (Bertram et al. U.S. Published Application 20070276507; McAllister et al. U.S. Pat. No. 7,112,218; Auger et al. U.S. Pat. No. 5,618,718; Niklason et al. U.S. Pat. No. 6,537,567); pressure-induced seeding (Torigoe et al. (2007) Cell Transplant., 16(7):729-39; Wang et al. (2006) Biomaterials. May; 27(13):2738-46); and electrostatic seeding (Bowlin et al. U.S. Pat. No. 5,723,324) may be used. In addition, a recent technique that simultaneously coats electrospun fibers with an aerosol of cells may be suitable for seeding or deposition (Stankus et al. (2007) Biomaterials, 28:2738-2746).

In one embodiment, the deposition of cells includes the step of contacting a scaffold with a cell attachment enhancing protein. In another embodiment, the enhancing protein is one or more of the following: fibronection, collagen, and MATRIGEL™. In one other embodiment, the scaffold is free of a cell attachment enhancing protein. In another embodiment, the deposition of cells includes the step of culturing after contacting a scaffold with a cell population. In yet another embodiment, the culturing may include conditioning by pulsatile and/or steady flow in a bioreactor.

Smooth muscle cell populations isolated from peritoneal tissue as described herein may then be seeded on a scaffold described herein. The peritoneal tissue may be omentum tissue.

The following is a representative example of a protocol for seeding omentum-derived smooth muscle cells on a scaffold. Omentum-derived smooth muscle cells may be expanded for several weeks (e.g., up to 7 weeks) to generate the quantity of cells required for seeding a scaffold. The density of cells suitable for seeding a scaffold is described below. Omentum-derived smooth muscle cells may be expanded for a number of passages before harvesting of cells for seeding of scaffolds to produce a construct. To prepare a scaffold for cell seeding, a suitable material (e.g., PGA felt) may be cut cut to size, sutured into the appropriate shape, and coated with material (e.g., PLGA). The scaffold may then be sterilized using a suitable method (e.g., ethylene oxide). On the day prior to cell seeding, the sterilized scaffold may be serially pre-wetted by saturation with 60% ethanol/40% D-PBS, 100% D-PBS, D-MEM/10% FBS or α-MEM/10% FBS followed by incubation in D-MEM/10% FBS or α-MEM/10% FBS at room temperature overnight. The scaffold can then be seeded with omentum-derived smooth muscle cells and the seeded construct matured in a humidified 37oC incubator at 5% CO2 until implantation in a subject (e.g., by day 7). Those of ordinary skill in the art will appreciate additional methods for preparing scaffolds for seeding of cells and seeding of cells onto scaffolds.

In one aspect, the present invention provides methods of preparing a construct having peritoneal-derived smooth muscle cells. In one embodiment, the method includes the steps of a) obtaining a human peritoneal tissue sample; b) isolating a smooth muscle cell population from the sample; c) culturing the cell population; and d) contacting the cell population with a shaped polymeric matrix cell construct. The human peritoneal tissue sample may be obtained from an autologous or non-autologous source. The human peritoneal tissue sample may be omentum tissue. In one other embodiment, the method further includes the step of detecting expression of a smooth muscle cell marker. In another embodiment, the expression is mRNA expression. In a further embodiment, the expression is polypeptide expression. In one embodiment, the polypeptide expression is detected by intracellular immunoflourescence.

In one embodiment, the scaffold comprises a cell population as described herein. In another embodiment, the scaffold consists essentially of a cell population as described herein. In one other embodiment, the scaffold consists of a cell population as described herein.

The first polymeric matrix or the second polymeric matrix, if any, or both, comprise at least one cell population deposited on or in a first surface of the first polymeric matrix, a first surface of the second polymeric matrix, or both, to form a construct of matrix or scaffold plus cells, wherein at least one cell population comprises substantially a muscle cell population. The muscle cell population is, e.g., a smooth muscle cell population. In a preferred embodiment, the first surface and the second surface are each the outer surface of the first and second polymeric matrices.

In another embodiment, the construct containing the matrix and cells is free of any other cell populations. In a preferred embodiment, the construct is free of urothelial cells.

These constructs are used to provide a luminal organ or tissue structures such as genitourinary organs, including for example, the urinary bladder, ureters and urethra, to a subject in need. The subject may require the reconstruction, augmentation or replacement of such organs or tissues. In one embodiment, the luminal organ or tissue structure is a bladder or portion thereof, and the polymeric matrix or scaffold has smooth muscle cells deposited on a surface of the matrix. The constructs may also be used to provide a urinary diversion or conduit, or a detrusor muscle equivalent.

In one aspect, the invention provides urinary diversion or conduit scaffolds or matrices that are seeded with a cell population described herein. Such scaffolds that have been seeded with a cell population and may be referred to herein as “constructs”. In one embodiment, the urinary diversion or bladder conduit construct is made up of one or more scaffolds as described herein and a cell population deposited on one or more surfaces of the one or more scaffolds as described herein.

In one aspect, the present invention provides urinary diversion constructs and methods of making and using the same. In one embodiment, the urinary diversion is for a defective bladder in a subject and includes (a) a first implantable, biocompatible construct comprising a tubular scaffold having a first end configured to connect to an abdominal wall section, a second closed end, and at least a first side opening configured to connect to a first ureter; and (b) a peritoneal-derived cell population, deposited on or in a surface of the scaffold. In another embodiment, the urinary diversion is for a defective bladder in a subject and includes (a) an implantable, biocompatible tubular scaffold adapted for temporary storage and passage of urine that comprises a first end configured to connect to an opening in the subject's abdominal wall, a second closed end, and at least a first side opening adapted to connect to a first ureter to allow passage of urine from the first ureter to the interior of the tubular scaffold; and (b) a peritoneal-derived cell population, deposited on or in a surface of the scaffold.

In one embodiment, the present invention provides a method of preparing a urinary diversion construct for a defective bladder in a subject in need that includes the steps of a) providing a first implantable biocompatible scaffold comprising a tubular scaffold having a first end configured to contact an abdominal wall section, a second closed end, and at least a first side opening configured to connect to a first ureter; and b) depositing a peritoneal-derived cell population on or in a first area of the scaffold to form a urinary diversion construct. In another embodiment, the method includes the steps of a) providing an implantable, biocompatible tubular scaffold adapted for temporary storage and passage of urine that comprises a first end configured to connect to an opening in the subject's abdominal wall, a second closed end, and at least a first side opening adapted to connect to a first ureter to allow passage of urine from the first ureter to the interior of the tubular scaffold; and b) depositing a peritoneal-derived cell population on or in a surface of the scaffold to form a urinary diversion construct.

In one aspect, the present invention provides muscle equivalent constructs that may be used to enhance an existing luminal organ or tissue structures such as genitourinary organs, including for example, the urinary bladder, to a subject in need. The subject may require expansion or treatment of such organs or tissues. In one embodiment, the luminal organ or tissue structure is a bladder or portion thereof, and the polymeric matrix or scaffold has smooth muscle cells deposited on a surface of the matrix. In one embodiment, the constructs are used to provide a detrusor muscle equivalent.

Those of ordinary skill in the art will appreciate there are several suitable methods for depositing cell populations upon matrices or scaffolds.

In one aspect, the constructs are suitable for implantation into a subject in need of a new organ or tissue structure. In one embodiment, the construct comprises a population of cells that produce the cytokine MCP-1. In another embodiment, the MCP-1 elicits the migration of the subject's or recipient's native mesenchymal stem cells to the site of implantation. In one embodiment, the migrating recipient native mesenchymal stem cells assist in the regeneration of the new organ or tissue structure.

In one other aspect, the invention provides scaffolds seeded with cells at particular cell densities. In one embodiment, a scaffold is seeded with a smooth muscle cell population at a cell density of about 20×10⁶ to about 30×10⁶ cells. In another embodiment, the cell density is about 1×10⁶ to about 40×10⁶, about 1×10⁶ to about 30×10⁶, about 1×10⁶ to about 10×10⁶, about 1×10⁶ to about 10×10⁶, or about 1×10⁶ to about 5×10⁶.

In a further embodiment, the cell density is about 20×10⁶ to about 98×10⁶ cells. In yet further embodiments, the cell density is about 21×10⁶ to about 97×10⁶, about 22×10⁶ to about 95×10⁶, about 23×10⁶ to about 93×10⁶, about 24×10⁶ to about 91×10⁶, about 25×1⁶ to about 89×10⁶, about 26×10⁶ to about 87×10⁶, about 28×10⁶ to about 85×10⁶, about 29×10⁶ to about 83×10⁶, about 30×10⁶ to about 80×10⁶, about 35×10⁶ to about 75×10⁶, about 40×10⁶ to about 70×10⁶, about 45×10⁶ to about 65×10⁶, or about 50×10⁶ to about 60×10⁶. In a preferred embodiment, the cell density is about 24×10⁶ to about 91×10⁶ cells.

In another embodiment, the cell density is about 2.5×10⁶ to about 40×10⁶, about 5×10⁶ to about 40×10⁶, about 7.5×10⁶ to about 35×10⁶, about 10×10⁶ to about 30×10⁶, about 15×10⁶ to about 25×10⁶, and about 17.5×10⁶ to about 22.5×10⁶. In another embodiment, the cell density is about 1×10⁶, about 2×10⁶, about 3×10⁶, about 4×10⁶, about 5×10⁶, about 6×10⁶, about 7×10⁶, about 8×10⁶, about 9×10⁶, about 10×10⁶, about 11×10⁶, about 12×10⁶, about 13×10⁶, about 14×10⁶, about 15×10⁶, about 16×10⁶, about 17×10⁶, about 18×10⁶, about 19×10⁶, about 20×10⁶, about 21×10⁶, about 22×10⁶, about 23×10⁶, about 24×10⁶, about 25×10⁶, about 26×10⁶, about 27×10⁶, about 28×10⁶, about 29×10⁶, about 30×10⁶, about 31×10⁶, about 32×10⁶, about 33×10⁶, about 34×10⁶, about 35×10⁶, about 36×10⁶, about 37×10⁶, about 38×10⁶, about 39×10⁶, about 40×10⁶, about 41×10⁶, about 42×10⁶, about 43×10⁶, about 44×10⁶, about 45×10⁶, about 46×10⁶, about 47×10⁶, about 48×10⁶, about 49×10⁶, about 50×10⁶, about 51×10⁶, about 52×10⁶, about 53×10⁶, about 54×10⁶, about 55×10⁶, about 56×10⁶, about 57×10⁶, about 58×10⁶, about 59×10⁶, about 60×10⁶, about 61×10⁶, about 62×10⁶, about 63×10⁶, about 64×10⁶, about 65×10⁶, about 66×10⁶, about 67×10⁶, about 68×10⁶, about 69×10⁶, about 70×10⁶, about 71×10⁶, about 72×10⁶, about 73×10⁶, about 74×10⁶, about 75×10⁶, about 76×10⁶, about 77×10⁶, abput 78×10⁶, about 79×10⁶, about 80×10⁶, about 81×10⁶, about 82×10⁶, about 83×10⁶, about 84×10⁶, about 85×10⁶, about 86×10⁶, about 87×10⁶, about 88×10⁶, about 89×10⁶, about 90×10⁶, about 91×10⁶, about 92×10⁶, about93×10⁶, about 94×10⁶, about 95×10⁶, about 96×10⁶, about 97×10⁶, about 98×10⁶, or about 99×10⁶.

In a further aspect, the invention provides scaffolds seeded with cells at particular cell densities per cm² of a scaffold. In one embodiment, the density is about 3,000 cells/cm² to about 15,000 cells/cm², about 3,500 cells/cm² to about 14,500 cells/cm², about 4,000 cells/cm² to about 14,000 cells/cm², about 4,500 cells/cm² to about 13,500 cells/cm², about 5,000 cells/cm² to about 13,000 cells/cm², about 4,500 cells/cm² to about 13,500 cells/cm², about 5,000 cells/cm² to about 13,000 cells/cm², about 5,500 cells/cm² to about 12,500 cells/cm², about 6,000 cells/cm² to about 12,000 cells/cm², about 6,500 cells/cm² to about 11,500 cells/cm², about 7,000 cells/cm² to about 11,000 cells/cm², about 7,500 cells/cm² to about 10,500 cells/cm², about 8,000 cells/cm² to about 10,000 cells/cm², about 7,500 cells/cm² to about 9,500 cells/cm², or about 8,000 cells/cm² to about 9,000 cells/cm². In a preferred embodiment, the density is about 3,000 cells/cm² to about 7,000 cells/cm², or about 9,000 cells/cm² to about 15,000 cells/cm².

In one aspect, the constructs of the present invention are adapted to provide particular features to the subject following implantation. In one embodiment, the constructs are adapted to provide regeneration to the subject following implantation. In another embodiment, the constructs are adapted to promote regeneration in a subject at the site of implantation. For example, following implantation, regenerated tissue may form from the construct itself at the site of implantation. In another embodiment, the construct may impart functional attributes to the subject following implantation. For example, a urinary diversion construct may be adapted to allow the passage of a subject's urine from a first ureter (e.g., first side opening) to the interior of the tubular scaffold, and/or adapted to provide temporary storage and passage of urine (e.g., tubular scaffold) out of a subject. In one embodiment, a urinary diversion construct may be adapted to provide an epithelialized mucosa upon implantation. In another embodiment, a construct may be adapted to provide homeostatic regulative development of a new organ or tissue structure in a subject.

### 6. Methods of Use

In one aspect, the present invention contemplates methods for providing a laminarily organized luminal organ or tissue structure to a subject in need of such treatment. In one embodiment, the subject may be in need of regeneration, reconstruction, augmentation, or replacement of an organ or tissue. In one embodiment, the method includes the step of providing a biocompatible synthetic or natural polymeric matrix shaped to conform to at least a part of the organ or tissue structure in need of an organ or tissue structure. The providing step may be followed by depositing at least one cell population that is not derived from the organ or tissue structure that is the subject of the reconstruction, augmentation or replacement. The depositing step may include culturing the cell population on the polymeric matrix. After depositing the cell population on the matrix to provide a construct, it can be implanted into a patient at the site of treatment for the formation of the desired laminarily organized luminal organ or tissue structure. In one embodiment, the laminarly organized luminal organ or tissue structure is a bladder or a part of a bladder.

In one other aspect, the present invention provides methods for providing a laminarily organized luminal organ or tissue structure to a subject in need. In one embodiment, the method includes the steps of a) providing a biocompatible synthetic or natural polymeric matrix shaped to conform to at least a part of the organ or tissue structure in need of said treatment; b) depositing on or in a first area of the polymeric matrix a cell population that is not derived from a native organ or tissue corresponding to the new organ or tissue structure; and c) implanting the shaped polymeric matrix cell construct into said the subject for the formation of laminarily organized luminal organ or tissue structure. In one other aspect, the present invention provides methods for providing a neo-bladder or portion thereof to a subject in need. In one embodiment, the method includes a) providing a biocompatible synthetic or natural polymeric matrix shaped to conform to a bladder or portion thereof; b) depositing a cell population that is not derived from the subject's bladder on or in a first area of the polymeric matrix; and c) implanting the shaped polymeric matrix cell construct into the subject for the formation of the neo-bladder or portion thereof. In another embodiment, the cell population of step b) of the methods described herein contains one or more peritoneal-derived smooth muscle cells having contractile function that are positive for a smooth muscle cell marker. In one other embodiment, the contractile function of the cell population is calcium-dependent. The SMCs may be derived from omentum.

In one embodiment, the methods of the present invention further include the step of wrapping the implanted conduit construct with the subject's omentum, mesentery, muscle fascia, and/or peritoneum to allow for vascularization.

In one other aspect, the present invention provides methods for providing a urinary diversion or conduit for a defective bladder in a subject in need. In one embodiment, the method for providing a urinary diversion to a subject in need includes the steps of (a) providing a biocompatible conduit scaffold; (b) depositing a first cell population on or in a first area of said scaffold, said first cell population being substantially a muscle cell population; and (c) implanting the scaffold of step (b) into said subject to form a conduit that allows urine to exit the subject. In another embodiment, the biocompatible material is biodegradable. In other embodiments, the biocompatible material is polyglycolic acid. In yet another embodiment, the first cell population is substantially a smooth muscle cell population.

In one embodiment, the method includes the step of providing a urinary diversion or conduit scaffold as described herein. In other additional embodiments, the urinary diversion or conduit scaffold is provided in multiple parts, such as a first, second, and third scaffold, as described herein. In another embodiment, the method further includes the step of depositing a cell population that is not derived from the defective bladder to form a urinary diversion or conduit construct. In one other embodiment, the depositing step may include culturing the cell population on the scaffold. In some embodiments, the methods further includes the step of implanting the urinary diversion construct into a patient in need. In another embodiment, the implantation is at the site of the defective bladder.

In one embodiment, an open end of the construct (e.g., a first end configured to connect to the abdominal wall) is anastomosed to the skin (ostomy) throught the abdominal or suprapubic wall to form a stoma or sphincter. In another embodiment, a catheter is inserted through stoma opening and into the lumen of the construct to provide urine outflow.

FIG. 10 illustrates a configuration for an implanted conduit construct.

In one other embodiment, the present invention provides a method of providing a urinary diversion for a defective bladder in a subject in need that includes the steps of a) providing a first implantable biocompatible scaffold comprising a tubular scaffold having a first end configured to connect to an abdominal wall section, a second closed end, and at least a first side opening configured to connect to a first ureter; and b) depositing a peritoneal-derived cell population on or in a first area of the scaffold to form a urinary diversion construct; and c) implanting the construct into the subject for the formation of the urinary diversion. In another embodiment, the method includes the steps of a) providing an implantable, biocompatible tubular scaffold adapted for temporary storage and passage of urine that comprises a first end configured to connect to an opening in the subject's abdominal wall, a second closed end, and at least a first side opening adapted to connect to a first ureter to allow passage of urine from the first ureter to the interior of the tubular scaffold; b) depositing a peritoneal-derived cell population on or in a surface of the scaffold to form a urinary diversion construct; and c) implanting the construct into the subject for the formation of the urinary diversion. In one other embodiment, the method includes the step of implanting into the subject a urinary diversion construct comprising (a) a tubular scaffold having a first end configured to contact an abdominal wall section, a second closed end, and at least a first side opening configured to connect to a first ureter; and (b) a peritoneal-derived cell population, deposited on or in a surface of the scaffold, for the formation of the urinary diversion.

In all embodiments, the urinary diversion scaffold may further comprise a second side opening configured to connect to a second ureter. In all embodiments, the first end may be configured to be positioned flush with the abdominal wall. In all embodiments, the first end may be configured to be sutured to the skin of the subject. In all embodiments, the first end may be configured to form a stoma. In all embodiments, the stoma may further comprise a stoma button. In all embodiments, the scaffold further comprises a washer ring configured to form a stoma. In all embodiments, the biocompatible scaffold is biodegradable. In all embodiments, the scaffold may comprise a material selected from the group consisting of polyglycolic acid, polylactic acid, and a copolymer of polyglycolic acid and polylactic acid. In all embodiments, the cell population is a smooth muscle cell population. In all embodiments, the diversion may be a replacement for the defective bladder. In all embodiments, the diversion may be temporary. In all embodiments, the diversion may be permanent. In all embodiments, the tubular scaffold may have a rectangular cross-section configuration or a triangular cross-section configuration, or a circular cross-section configuration. In all embodiments, the diversion may be free of urothelial cells. In all embodiments, the methods of the present invention may provide a neo-urinary conduit characterized by urinary-like tissue regeneration. In all embodiments, the regenerated tissue may be characterized by the presence of one or more of the following: urothelium, lamina propria, and smooth muscle bundles. In all embodiments, the regenerated tissue can be observed at one or more of the following: ureter-conduit junction (UCJ), cranial portion of the conduit, and mid-atrium portion of the conduit. In all embodiments, the regenerated tissue may be characterized by the presence of one or more of the following: mucosa, submucosa, and smooth muscle with a fibrovascular stroma. In all embodiments, the regenerated tissue is continuous urothelium with underlying smooth muscle. In all embodiments, the urinary conduit forms an epithelialized mucosa upon implantation.

In another embodiment, the methods of the present invention further include the step of monitoring the conduit for the presence of an obstruction following implantation of the urinary diversion construct. The obstruction may be caused by the build-up of detritis. The method may further include the step of removing detritis from the lumen of the conduit if an obstruction is detected.

In one aspect, the present invention provides a urinary diversion to a subject in need on a temporary basis. In one embodiment, a temporary urinary diversion or conduit construct is implanted into a subject to form a stoma opening, and a catheter or other device is temporarily inserted through the stoma to the lumen of the conduit construct. A temporary conduit provides the advantage of allowing urine to exit the subject while a permanent solution to the defective bladder is attempted. For example, the implantation of a conduit construct could be performed prior to, following, or simultaneous with the implantation of a neo-bladder construct seeded with a cell population (see for example Bertram et al. supra). FIG. 11 shows an example of the implanted components of a temporary urinary diversion construct.

In one embodiment, the methods of the present invention further include the step of wrapping the implanted urinary diversion or conduit construct with the subject's omentum, mesentery, muscle fascia, and/or peritoneum to allow for vascularization.

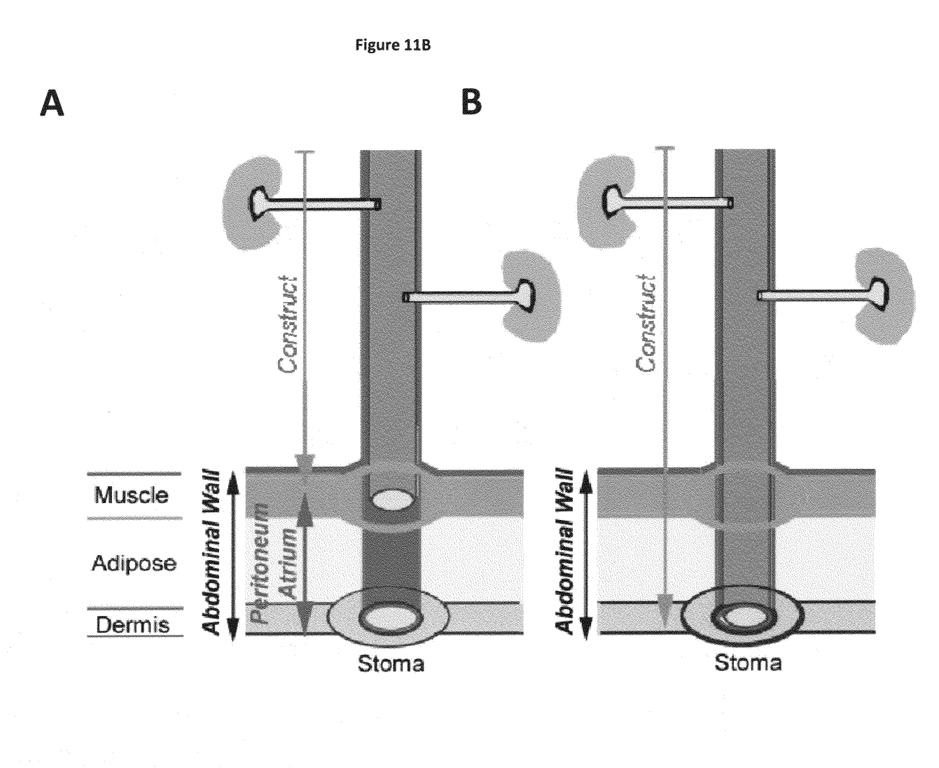

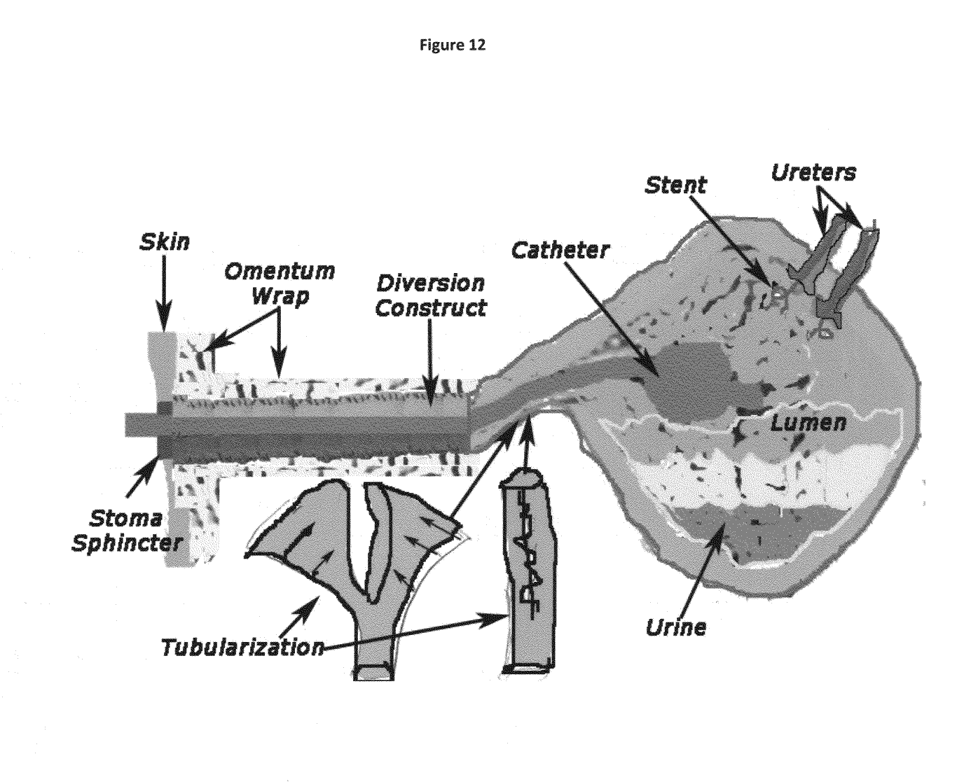

In one aspect, the present invention provides a urinary diversion to a subject in need on a permanent basis. FIG. 12 shows an example of the implanted components of a permanent urinary diversion construct.

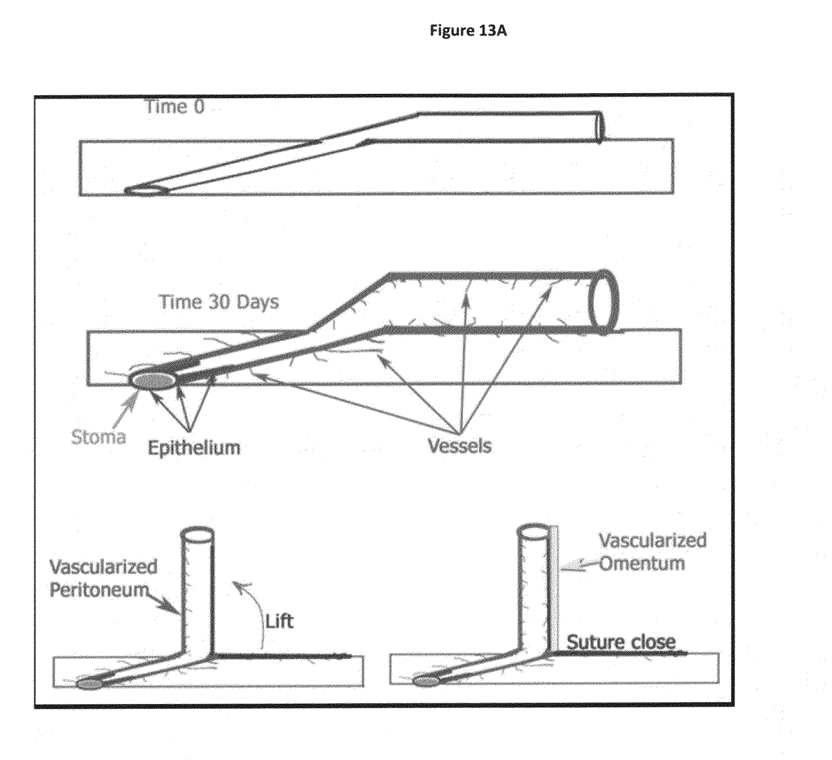

In one embodiment, the constructs described herein may be used for a prostatic urethra replacement and urinary diversion. Such a procedure is necessary for subjects requiring a radical prostatectomy to remove the prostatic urethra. In other embodiments, the constructs may be used for a percutaneous diversion tube to form a continent tube with a valve-like kink. In an additional embodiment, the constructs may be used as a bladder neck sling and wrapping materials used in bladder neck surgery and urinary outlets with continent channels or catherizable openings. Examples of such embodiments are depicted in FIG. 13.

Urine exits the body via the urethral meatus, a distinct structure incorporating features that defend the opening against local and/or ascending infections, and emptying in the vaginal vestibule in females and fossa navicularis in males. Specifically, the mucocutaneous in this region is a non-keratinized stratified squamous epithelium composed of glycogen-rich cells that provide substrate for a protective endogenous lactobacteria flora. Also, as the epithelium nears the skin it is associated with acid-phosphatase activity and lysozyme-like immunoreactivity indicative of the presence of macrophages that secrete bactericidal compounds (Holstein A F et al. (1991) Cell Tissue Res 264: 23).

In one aspect, the urinary diversion or neo-urinary conduit (NUC) constructs described herein may lead to the formation of a native-like transition between urinary mucosa and skin epithelium that has the structural features of mucocutaneous regions observed in native urethras. The transition region may be referred to as an epithelialized mucosa. In one embodiment, the construct is adapted to form an epithelialized mucosa upon implantation. In one embodiment, the epithelialized mucosa comprises a vestibular region and a mucocutaneous region. In another embodiment, the vestibular region is adjacent to the mucocutaneous region. In another embodiment, the mucocutaneous region is located at the stromal end of the construct connected to the abdominal wall and skin of the subject. In general, naturally-occuring mucocutaneous regions are characterized by the presence of mucosa and cutaneous skin and typically exist near the orifices of the body where the external skin ends and the mucosa that covers the inside of the body starts. The epithelialized mucosa provided by the constructs and methods of the present invention develops at the first end of the urinary diversion construct following implantation into the subject. In a further embodiment, the epithelialized mucosa is characterized by the presence of an epithelium that first appears in the vestibular region and gradually expands or increases through the mucocutaneous region towards the stomal end of the construct. In another embodiment, the epithelium is characterized by expression of an epithelial cell marker. In a further embodiment, the epithelial cell marker is cytokeratin. The cytokeratin may be one or more of the cytokeratins known in the art including, without limtation, cytokeratins 1 through 19. In one other embodiment, the cytokeratin is detectable with AE-1/AE3 antibody.

The ability of the constructs described herein to form an epithelialized mucosa provides a solution to the major challenge of achieving urinary diversion via an abdominal stoma. It is accepted that the longevity of percutaneous devices is often hampered by exit-site infection (Knabe C et al. (1999) Biomaterials 20: 503). Percutaneous devices such as catheters, cannulas, prosthetic attachments, and glucose sensors, regardless of their intended medical goal, penetrate the skin, disrupt its protective barrier, and create a sinus tract for bacterial invasion (Isenhath SN et al. (2007) J Biomed Mater Res A 83: 915). Breakdown of the product-skin interface due to improper epidermal healing, lack of biocompatibility, or mechanical stresses can cause additional failure risks (von Recum A F and Park J B. (1981) Crit Rev Bioeng 5:37).

In another aspect, the urinary diversion constructs through interaction with the tissue of a recipient regenerate a tubular organoid. In one embodiment, the interaction of the construct with the recipient tissue is by transabdominal-percutaenous placement. In one other embodiment, the tubular organoid allows the flow of urine from the ureters to outside of the recipient. Urine flows out of the recipient while maintaining native-like functional properties found in bladders, urethras, and stomas (i.e., a meatus or opening). The muco-cutaneous junction resembles a junction found at the anterior urethra's opening; at the vaginal vestibule and fossa navicularis, of the human female and male, respectively. These natural junctions are covered by mucosal zones critical to wet-dry surfaces that may provide protection against ascending infections. The squamous epithelium of these mucosal zones is 1) glycogen-rich, 2) secretory (able to release enzymes and bactericidal agents), and 3) phagocytic; and can rapidly migrate to injured surfaces.

Grafting of scaffolds to an organ or tissue to be enlarged can be performed according to the methods described in the Examples or according to art-recognized methods. The matrix or scaffold can be grafted to an organ or tissue of the subject by suturing the graft material to the target organ.

The described techniques may be used to expand an existing laminarily organized luminal organ or tissue structure in a patient in need of such treatment. For example, an existing laminarily organized luminal organ or tissue structure may be enlarged by providing a polymeric matrix or scaffold shaped to conform to at least a part of the organ or tissue structure in need of said treatment and of a sufficient size to be laparoscopically implanted, depositing a cell population that is not derived from the organ or tissue structure on or in a first area of said polymeric matrix; and laparoscopically implanting the shaped polymeric matrix construct into said patient at the site of said treatment such that the existing laminarily organized luminal organ or tissue structure is expanded.

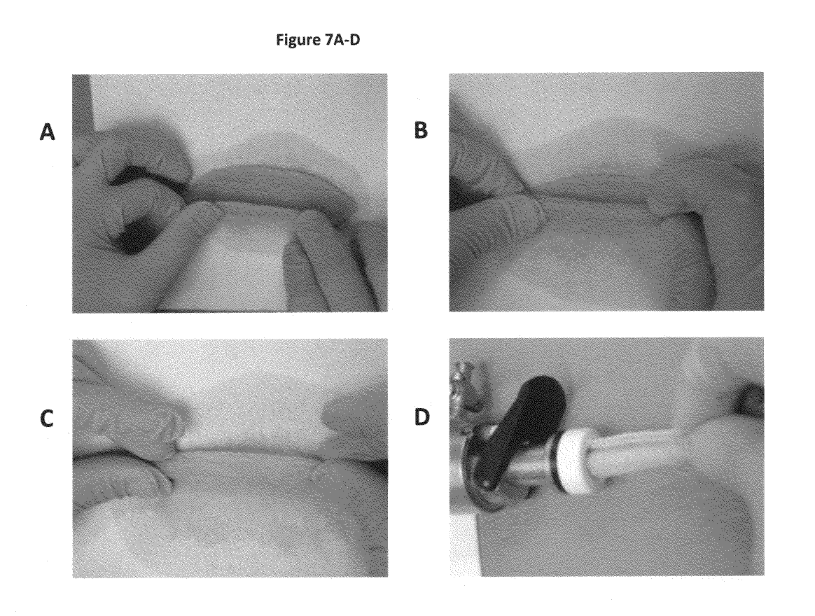

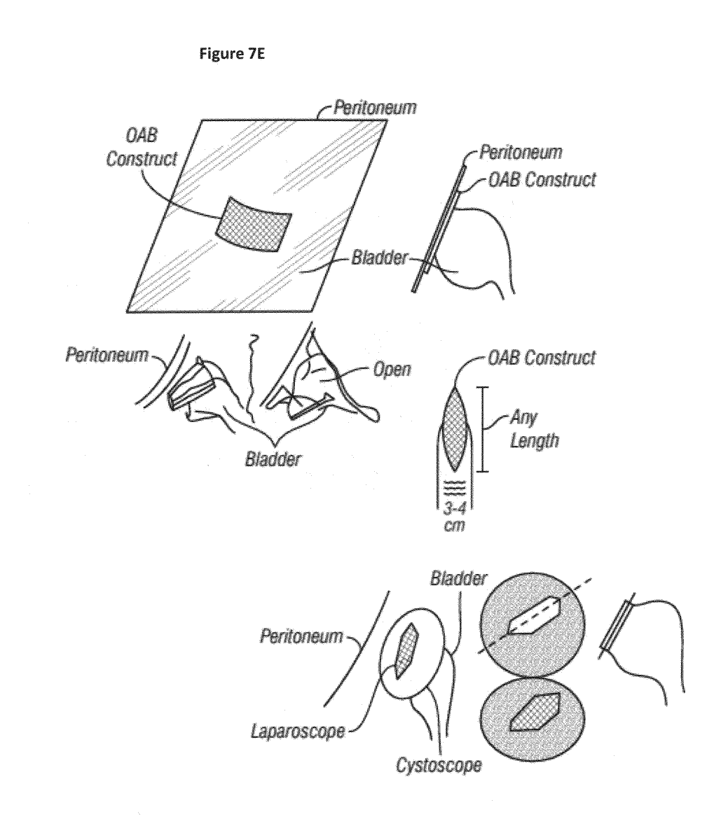

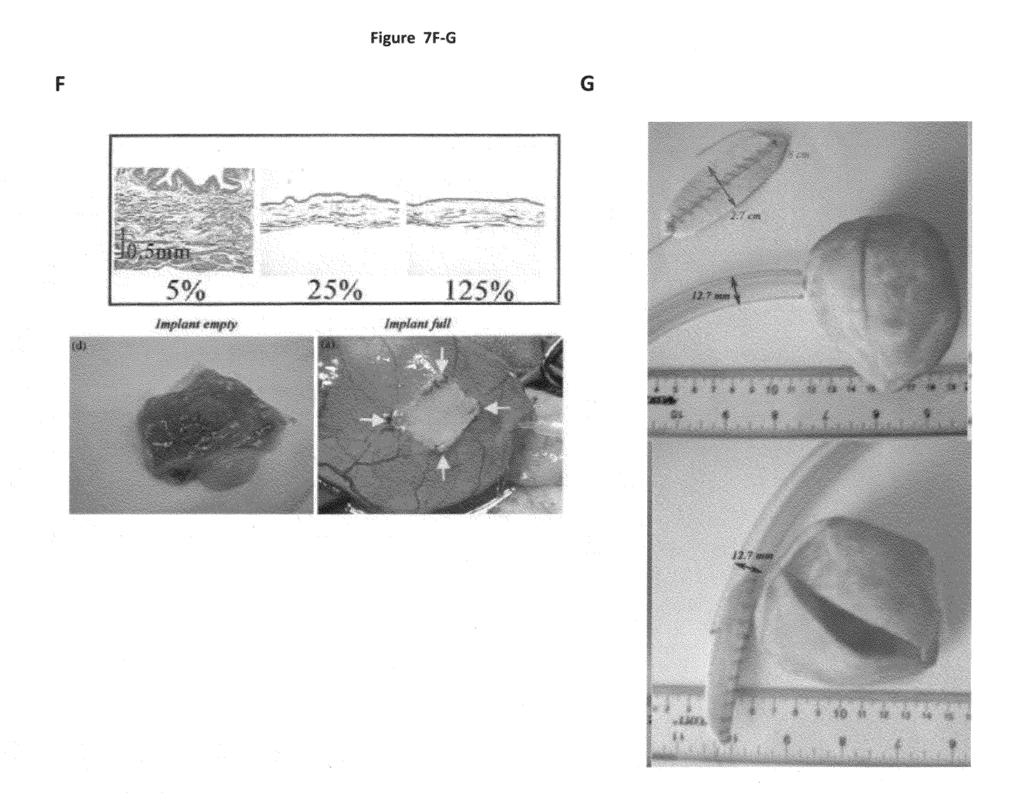

FIG. 7e depicts possible surgical methods for the implantation of a muscle equivalent scaffold described herein. FIG. 7f depicts implantation sites on an empty and full bladder. FIG. 7g depicts a urinary bladder model with surgical slit showing ellipsoid created upon sectioning of surface. A plastic tube may be used as a model of the limited space available in order to pass the folded or rolled polymeric matrices or scaffolds of the invention.

The described techniques may also be used to increase bladder volumetric capacity in a patient in need of such treatment. For example, bladder volumetric capacity may be increased by providing a biocompatible synthetic or natural polymeric matrix shaped to conform to at least a part of the organ or tissue structure in need of said treatment and of a sufficient size to be laparoscopically implanted; depositing a cell population that is not derived from the organ or tissue structure on or in a first area of said polymeric matrix; and laparoscopically implanting the shaped polymeric matrix construct laparoscopically into said patient at the site of said treatment such that bladder volume capacity is increased. In one embodiment, the matrix or scaffold of the instant invention is suitable for increasing bladder volume capacity about 50 mL. In other embodiments, the matrix or scaffold of the instant invention is suitable for increasing bladder volume capacity about 100 mL. In other embodiments, the matrix or scaffold of the instant invention is suitable for increasing bladder volume capacity about 60, about 70, about 80, or about 90 mL.

The described techniques may further be used to expand a bladder incision site in a patient in need of such treatment. For example, a bladder incision site may be expanded by providing a biocompatible synthetic or natural polymeric matrix shaped to conform to at least a part of the organ or tissue structure in need of said treatment and of a sufficient size to be laparoscopically implanted; b) depositing a cell population that is not derived from the organ or tissue structure on or in a first area of said polymeric matrix; and c) laparoscopically implanting the shaped polymeric matrix construct laparoscopically into said patient at the site of said treatment such that the bladder incision site is expanded.

Another non-limiting use of the invention includes methods for the treatment of urinary incontinence in a patient in need of such treatment. For example, urinary incontinence may be treated by providing a biocompatible synthetic or natural polymeric matrix shaped to conform to at least a part of the organ or tissue structure in need of said treatment and of a sufficient size to be laparoscopically implanted; depositing a cell population that is not derived from the organ or tissue structure on or in a first area of said polymeric matrix; and laparoscopically implanting the shaped polymeric matrix construct laparoscopically into said patient at the site of said treatment such that bladder volume capacity is increased.

In one embodiment, the scaffolds, cell populations, and methods described herein may further be used for the preparation of a medicament useful in the treatment of a disorder described herein. The disorders include any condition in a subject that requires the regeneration, reconstruction, augmentation or replacement of laminarly organized luminal organs or tissue structures. In another embodiment, the organ or tissue structure is a bladder or a part of the bladder.

In another embodiment, the cells deposited on the implanted construct produce MCP-1 and release it at the site of implantation, which stimulates native mesenchymal stem cells (MSCs) to migrate to the site of implantation. In one other embodiment, the native MSCs facilitate and/or enhance regeneration of the implanted construct at the site of implantation.

In one embodiment, the cell population deposited is a smooth muscle cell (SMC) population derived from peritoneal tissue as described herein. The peritoneal tissue may be omentum. In another embodiment, the SMC population includes at least one cell that has contractile function and is positive for a smooth muscle cell marker, such as myocardin, alpha-smooth muscle actin, calponin, myosin heavy chain, BAALC, desmin, myofibroblast antigen, SM22, vimentin and any combination thereof. In other embodiments, the SMC population includes at least one cell that demonstrates myocardin (MYOCD) expression. The MYOCD expression may be expression of a nucleic acid encoding a MYCOD polypeptide or a MYOCD polypeptide. In another embodiment, the contractile function of the SMC is calcium-dependent. In one embodiment, the laminarily organized luminal organ or tissue structure that is the subject of reconstruction, augmentation or replacement is a bladder or a portion of a bladder. In another embodiment, the polymeric matrix is free of urothelial cells.

In all embodiments, the methods of the present invention utilize a construct for implantation that is based upon a bladder replacement scaffold, a bladder augmentation scaffold, a bladder conduit scaffold, or a detrusor muscle equivalent scaffold that has been seeded with a cell population as described herein.

In another embodiment, the methods for the regeneration, reconstruction, augmentation or replacement of laminarly organized luminal organs or tissue structures described herein include the steps of a) providing a biocompatible synthetic or natural polymeric matrix shaped to conform to at least a part of the luminal organ or tissue structure in need of said treatment; b) depositing a first cell population on or in a first area of said polymeric matrix at a cell density described herein, said first cell population being substantially a muscle cell population; and c) implanting the shaped polymeric matrix cell construct into said patient at the site of said treatment for the formation of the laminarily organized luminal organ or tissue structure. In one other embodiment, the laminarily organized luminal organ or tissue structure formed in vivo exhibits the compliance of natural bladder tissue.

In one other aspect, the present invention provides methods for the regeneration of a neo-bladder following implantation into a subject in need thereof based upon biomechanical stimulation or cycling. In one aspect, the methods are suitable for use in promoting the regeneration of an implanted neo-bladder construct that has been implanted for the augmentation or replacement of a bladder or a portion of a bladder. In one embodiment, the neo-bladder construct is formed from seeding cells on a neo-bladder matrix or scaffold. In another embodiment, the neo-bladder scaffold is a bladder replacement scaffold, a bladder augmentation scaffold, a bladder conduit scaffold, or a detrusor muscle equivalent scaffold.

In one aspect, the method of the present invention applies to implanted neo-bladder constructs formed from seeding neo-bladder scaffolds with at least one cell population. In one embodiment, the cell-seeded polymeric matrix (or matrices) is a bladder replacement scaffold, a bladder augmentation scaffold, a bladder conduit scaffold, or a detrusor muscle equivalent scaffold. In one embodiment, the at least one cell population comprises substantially a muscle cell population. In another embodiment, the muscle cell population may be a smooth muscle cell population. Different densities of cells for seeding may be appropriate as described herein.

In one aspect, the methods of the present invention are performed at different times and for different durations following the implantation of the neo-bladder. In one embodiment, the cycling is performed on a daily basis over a period of time, on a weekly basis over a period of time, or every other week. In another embodiment, the duration of the daily cycling regimen is about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, or longer than 14 weeks.

In one embodiment, a daily cycling protocol for a subject may include the steps of filling the neo-bladder for about an hour, draining the filled neo-bladder for about an hour, and allowing the neo-bladder to drain freely, typically overnight. This protocol can be performed on day one of the cycling regimen in the subject. This daily sequence can be performed for a number of consecutive days after the first day. In one embodiment, the cycling protocol may be performed on a day after day one in which the duration of the filling step is increased to about two hours, about three hours, about four hours, or more than about four hours. In another embodiment, the filling and draining steps may be repeated more than once daily before allowing the neo-bladder to drain freely.

In another embodiment, the subjects are catheterized post-implantation, and the cycling time is controlled by clamping and unclamping the subject's catheter.

Those of ordinary skill in the art will appreciate that additional cycling regimens are contemplated herein.

An example of a cycling protocol is as follows. Following implantation of a neo-bladder construct formed by seeding a neo-bladder matrix or scaffold with cells as described herein, cycling will be performed every 2 weeks (14±2 day intervals) starting approximately 1 month after implantation and continuing until approximately Day 90. Cycling will be completed after certain types of assessment, such as compliance measurement of the implanted neo-bladder, but before other types of assessment such as fluoroscopic imaging. Cycling will be performed by re-inflating the bladder with sterile saline (warmed by incubator) after the completion of compliance measurement at a rate of 10-25 mL/min. The cycling will be repeated at least 5-10 times. The starting pressure of 0-10 mmHg will be achieved and recorded along with the start time. Time, volume of isotonic solution delivered, and the pressure obtained will be recorded for each cycle at the time leakage is observed around the catheter (a.k.a. leak point), or when the volume delivered is equal to that of the compliance measurement just performed, whichever comes first.

In one embodiment, the present invention provides a method of promoting regeneration of a neo-bladder implanted in a subject that includes the steps of (a) filling the implanted neo-bladder with a fluid; (b) emptying the filled neo-bladder of step (a). In another embodiment, the method includes step (c) repeating steps (a) and (b). In one other embodiment, the method is commenced within the first 2 weeks post-implantation. In one embodiment, the steps (a) and (b) are performed once daily, once weekly, or once every other week. In some other embodiments, the filling step (a) is performed for about one hour and the emptying step (b) is performed for about one hour. In yet another embodiment, steps a) and b) are performed at least until about six weeks post-implantation. In one other embodiment, steps a) and b) are not performed for more than about ten weeks post-implantation. In another embodiment, steps a) and b) are performed for more than about ten weeks post-implantation. In other embodiments, the filling comprises expanding the neo-bladder. In another embodiment, the regeneration comprises an increase in the capacity of the neo-bladder as compared to a neo-bladder in a subject that has not undergone cycling. In one other embodiment, the regeneration comprises an increase in compliance of the neo-bladder as compared to a neo-bladder in a subject that has not undergone cycling. In other embodiments, the regeneration comprises an increase in extracellular matrix development in the neo-bladder as compared to a neo-bladder in a subject that has not undergone cycling. In one embodiment, the increase in extracellular matrix development comprises the development of elastin fibers.

In one other aspect, the present invention concerns methods for providing homeostatic regulative development of neo-bladders in mammals such that implanted neo-bladders are responsive to the needs of the recipient. In one embodiment, the implanted neo-bladder grows to a size proportionate to the recipient. In another embodiment, the methods for providing homeostatic regulative development of a neo-bladder in a subject include the steps of (a) providing a biocompatible polymeric scaffold; (b) depositing an a first cell population on or in a first area of said scaffold, said first cell population being substantially a muscle cell population; and (c) implanting the scaffold of step (b) into said subject to establish homeostatic regulative development. In one other embodiment, the homeostatic regulative development comprises restoration of organ size and structure. In another embodiment, the homeostatic regulative development comprises neo-bladder capacities proportionate to body weight. In one embodiment, the proportionate neo-bladder capacity is achieved at about four months post-implantation. In another embodiment, the method for providing homeostatic regulative development of a neo-bladder in a subject includes the step of monitoring the state of homeostatic regulative development or progress of the implanted neo-bladder. The monitoring may include a cystogram procedure to show the position and shape of the implanted neo-bladder, and/or a measurement of urodynamic compliance and capacity.

In another aspect, the invention provides methods for prognostic evaluation of a patient following implantation of a new organ or tissue structure. In one embodiment, the method includes the step of detecting the level of MCP-1 expression in a test sample obtained from said subject; (b) determining the expression level in the test sample to the level of MCP-1 expression relative to a control sample (or a control reference value); and (c) predicting regenerative prognosis of the patient based on the determination of MCP-1 expression levels, wherein a higher level of expression of MCP-1 in the test sample, as compared to the control sample (or a control reference value), is prognostic for regeneration in the subject.

In another aspect, the invention provides methods for prognostic evaluation of a patient following implantation of a new organ or tissue structure in the patient, the methods comprising: (a) obtaining a patient biological sample; and (b) detecting MCP-1 expression in the biological sample, wherein MCP-1 expression is prognostic for regeneration in the patient. In some embodiments, increased MCP-1 expression in the patient biological sample relative to a control sample (or a control reference value) is prognostic for regeneration in the subject. In some embodiments, decreased MCP-1 expression in the patient sample relative to the control sample (or control reference value) is not prognostic for regeneration in the subject. The patient sample may be a test sample comprising a bodily fluid, such as blood or urine.

In some embodiments, the determining step comprises the use of a software program executed by a suitable processor for the purpose of (i) measuring the differential level of MCP-1 expression in a test sample and a control; and/or (ii) analyzing the data obtained from measuring differential level of MCP-1 expression in a test sample and a control. Suitable software and processors are well known in the art and are commercially available. The program may be embodied in software stored on a tangible medium such as CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner

Following the determining step, the measurement results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. In some embodiments, the assays will be performed or the assay results analyzed in a country or jurisdiction which differs from the country or jurisdiction to which the results or diagnoses are communicated.

In a preferred embodiment, a prognosis, prediction and/or treatment recommendation based on the level of MCP-1 expression measured in a test subject having a differential level of MCP-1 expression is communicated to the subject as soon as possible after the assay is completed and the prognosis and/or prediction is generated. The results and/or related information may be communicated to the subject by the subject's treating physician. Alternatively, the results may be communicated directly to a test subject by any means of communication, including writing, electronic forms of communication, such as email, or telephone. Communication may be facilitated by use of a computer, such as in case of email communications. In certain embodiments, the communication containing results of a prognosit test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, prognosis and/or prediction of regeneration, and communicating of assay results or prognoses, may be carried out in diverse (e.g., foreign) jurisdictions.

In another aspect, the prognostic methods described herein provide information to an interested party concerning the success of the implantation, and the rehabilitation/treatment protocol for regeneration. In one embodiment, the methods include the steps of detecting the level of MCP-1 expression in a test sample obtained from said subject; (b) determining the expression level in the test sample to the level of MCP-1 expression relative to a control sample (or a control reference value); and (c) predicting regenerative prognosis of the patient based on the determination of MCP-1 expression levels, wherein a higher level of expression of MCP-1 in the test sample, as compared to the control sample (or a control reference value), is indicative of the state of regeneration of a new organ or tissue structure.

Generally, as used herein, regeneration prognosis encompasses the forecast or prediction of any one or more of the following: development or improvement of a functional bladder after bladder replacement or augmentation through implantation of a construct described herein, development of a functional urinary diversion after implantation of a construct described herein, development of bladder capacity or improved bladder capacity after implantation of a construct described herein, or development of bladder compliance or improved bladder compliance after implantation of a construct described herein.

In all embodiments, the methods of providing a laminarily organized luminal organ or tissue structure to a subject in need of such treatment as described herein may include the post-implantation step of prognostic evaluation of regeneration as described above.

In all embodiments, the present invention relates to methods for providing a new organ or tissue structure to a subject in need that include certain post-implantation monitoring steps. In one embodiment, the effect and performance of an implanted constructs is monitored, such as through ultrasound imaging, pyelogram, as well as urine and blood analysis at different time-points after implantation.

### 7. Kits

The instant invention further includes kits comprising the polymeric matrices and scaffolds of the invention and related materials, and/or cell culture media and instructions for use. The instructions for use may contain, for example, instructions for culture of the cells or administration of the cells and/or cell products. The instructions for use may also contain instructions for pre-treating, folding or otherwise preparing the polymeric matrices and scaffolds of the invention for laparoscopic implantation.

In one embodiment, the present invention provides a kit comprising a scaffold as described herein and instructions. In another embodiment, the scaffold of the kit is one or more of the following: a bladder augmentation scaffold, a bladder replacement scaffold, a urinary conduit scaffold, or a muscle equivalent scaffold.

### 8. Reports

The methods of this invention, when practiced for commercial purposes generally produce a report or summary of the regenerative prognosis. The methods of this invention will produce a report comprising a prediction of the probable course or outcome of regeneration before and after any surgical procedure to provide a construct described herein. The report may comprise information on any indicator pertinent to the prognosis. The methods and reports of this invention can further include storing the report in a database. Alternatively, the method can further create a record in a database for the subject and populate the record with data. In one embodiment the report is a paper report, in another embodiment the report is an auditory report, in another embodiment the report is an electronic record. It is contemplated that the report is provided to a physician and/or the patient. The receiving of the report can further include establishing a network connection to a server computer that includes the data and report and requesting the data and report from the server computer. The methods provided by the present invention may also be automated in whole or in part.

The following examples are offered for illustrative purposes only, and are not intended to limit the scope of the present invention in any way.

All patent, patent applications, and literature references cited in the present specification are hereby incorporated by reference in their entirety.

### EXAMPLES

### Example 1

### Omentum as a Source of SMCs

Smooth muscle cells have been successfully isolated from canine and porcine omentum. To begin with, omental-derived smooth muscle cells were isolated from canine or porcine omentum tissue by washing the tissue in buffered saline to remove surface contaminants As illustrated by the diagram in FIG. 14 the omentum was then subjected to a series of enzyme digestion and centrifugation steps to yield isolated omental-derived cells for further culturing and characterization. Briefly, the washed omentum tissue was digested with a solution of 0.1% collagenase I (Worthington Biochemical, Lakewood, NJ) and 1% BSA (Sigma, St. Louis, Mo.) in DMEM-HG (Invitrogen, Carlsbad, Calif.) at 37° C. for 1 hour. To aid tissue digestion, the solution was agitated in 50 ml conical tubes placed on a platform shaker at a speed of 50 and a tilt of 15 during the 1 hour incubation at 37° C. Following digestion, the solution was centrifuged at 300×g for five minutes and the resulting pelleted material containing the omental-derived cells was then washed by a series of steps involving resuspension in phosphate buffered saline (PBS) at PBS1% and centrifugation to pellet at 300×g for five minutes to remove the fatty plug and other non-essential tissue debris. After washing, the cells were re-suspended in culture medium comprising DMEM+10% FBS and plated on T-flasks. Culturing was performed in commercially available medium supplemented with cytokines and growth factors for the purpose of ex vivo expansion. When appropriate cell numbers were reached by subsequent cell passaging, observations of cell morphology were made and an aliquot was fixed and processed for immunodetection of expressed smooth muscle cell proteins. For comparative analyses, cells were also isolated from bladder smooth muscle layer by culturing tissue explants using the protocol described in Atala et al., (J. Urol. 150: 608, 1993); Cilento et al., (J. Urol. 152: 655, 1994); and Atala U.S. Pat. No. 6,576,019 (incorporated herein by reference in its entirety). Those of ordinary skill in the art will appreciate other suitable methods of the isolation no cells.

### Example 2

### SMC Characterization

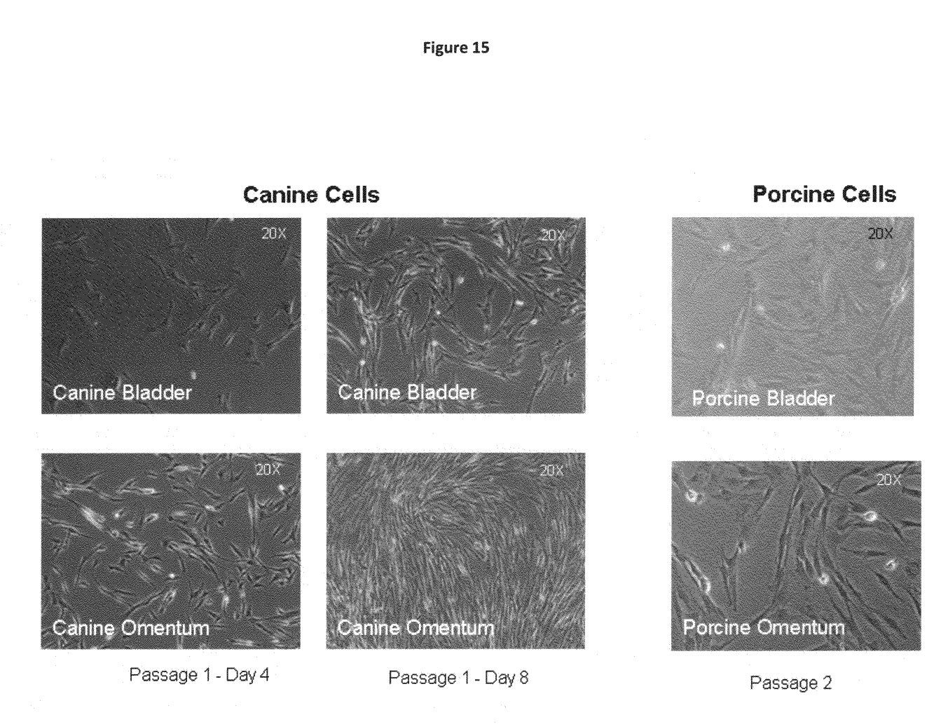

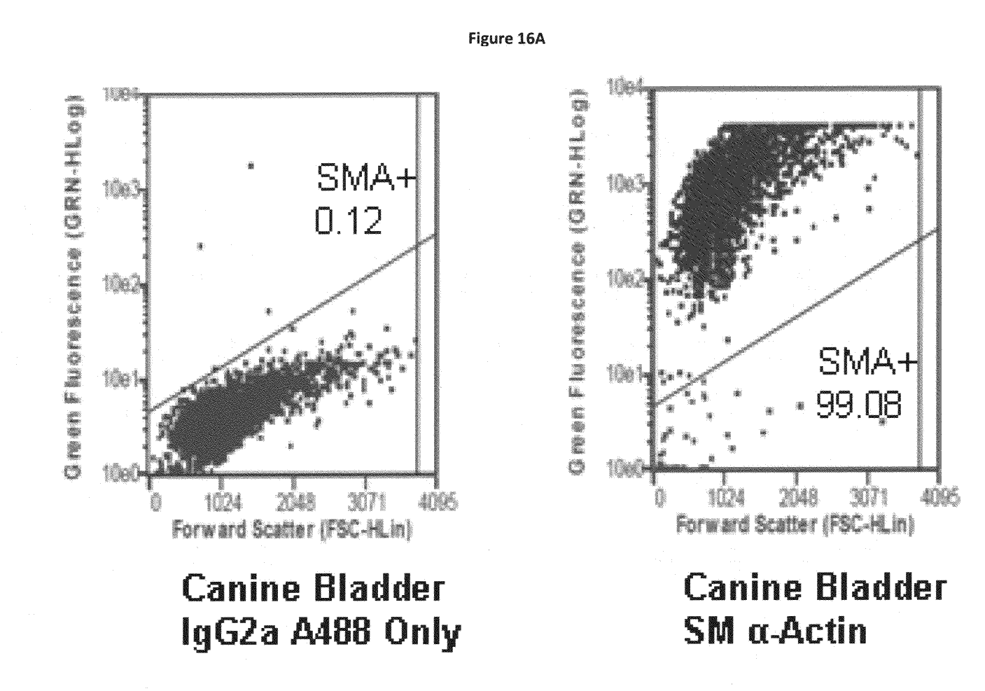

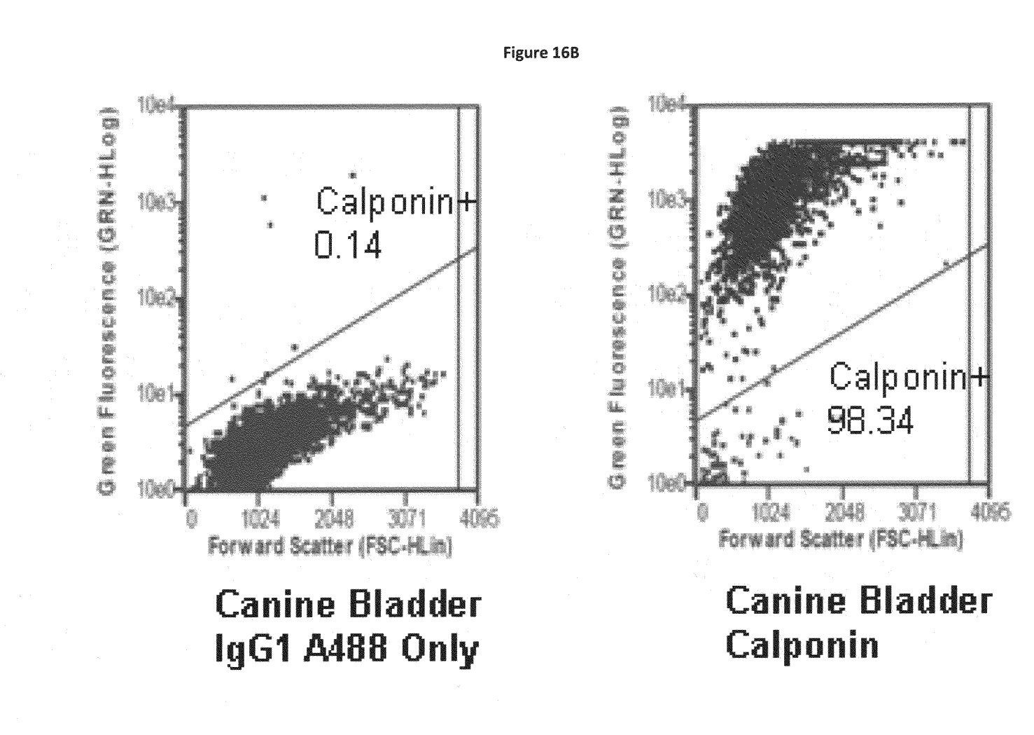

Morphology. FIG. 15 shows cell morphology of canine- and porcine-derived omentum cells compared with canine- and porcine-derived bladder cells. Cell morphology of canine and porcine bladder smooth muscle and omentum-derived cells reveals similar if not identical morphology when grown in DMEM+10% FBS. Cells are spindle shaped and elongated, with evidence of whirling and hill-and-valley formation. Thus, omental-derived appear to be smooth-muscle cell like in their morphology.

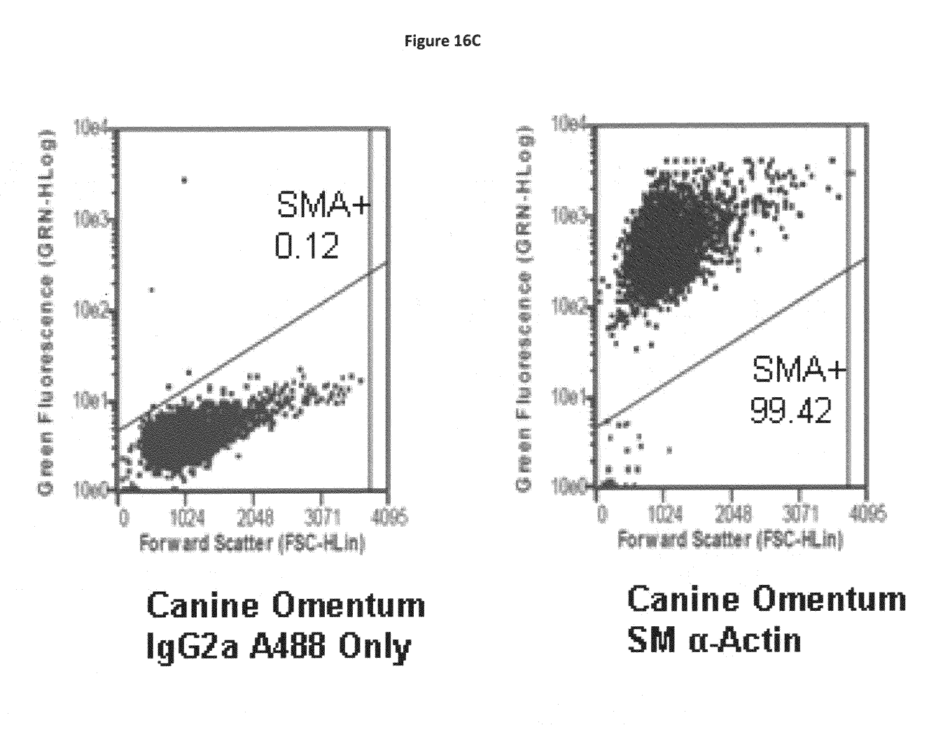

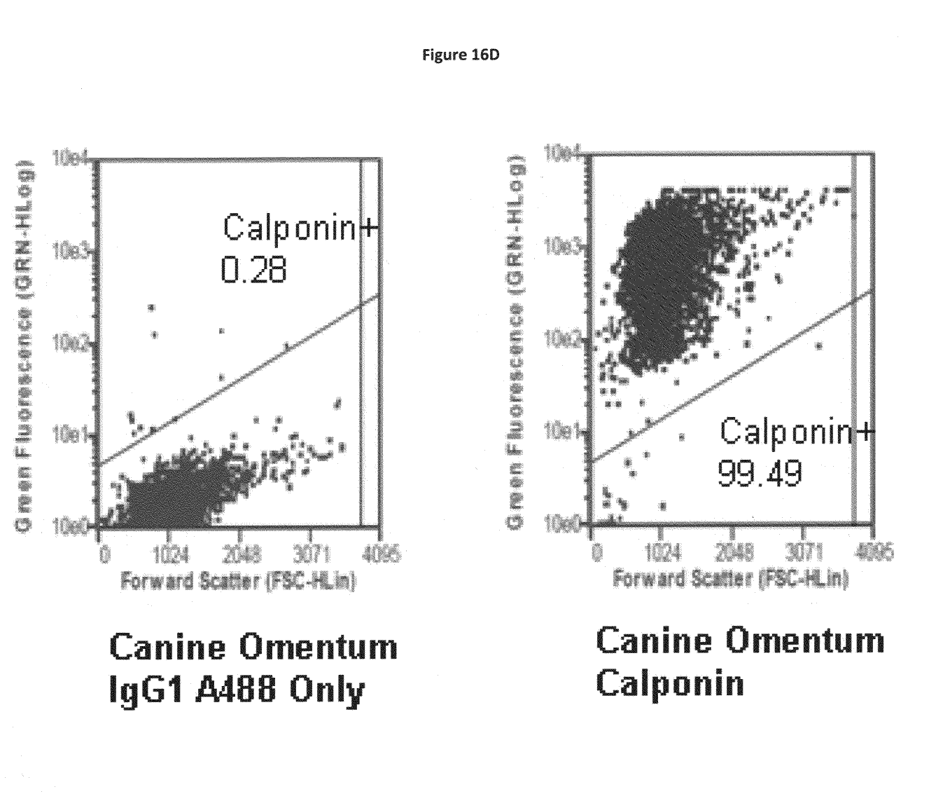

Fluorescence Activated Cell Sorting (FACS) analysis of cell surface markers. FIG. 16A-D illustrates a characterization of the cellular phenoytpe by FACS analysis, which shows that canine-derived omentum cells are positive for the smooth muscle cell markers alpha-actin and calponin. Briefly, 0.5×10⁶-1×10⁶ cells per data point were fixed in 2% paraformaldehyde and the Fc receptors were blocked to prevent non-specific binding. Cells were then incubated with antibody to smooth muscle cell alpha-actin (SMA) and calponin as recommended by the manufacturer. Isotype control antibody (IgG1 or IgG2a) was used as a negative control. Subsequent to final washing (PBS, 0.1% Triton X-100), antigen detection was performed utilizing the BD FACS Aria 1 or Guava EasyCyte Mini Express Assay system using the appropriate fluorescent channel. A minimum of 5,000-10,000 events were acquired from each sample. Similar to bladder smooth muscle cells (FIGS. 16 A and B), greater than 98% of cells isolated from omentum (FIGS. 16C and D) express the smooth muscle cell markers alpha-actin and calponin. Thus, omental-derived cells have the same phenotype as smooth muscle cells isolated from the bladder with respect to characteristic cell surface markers.

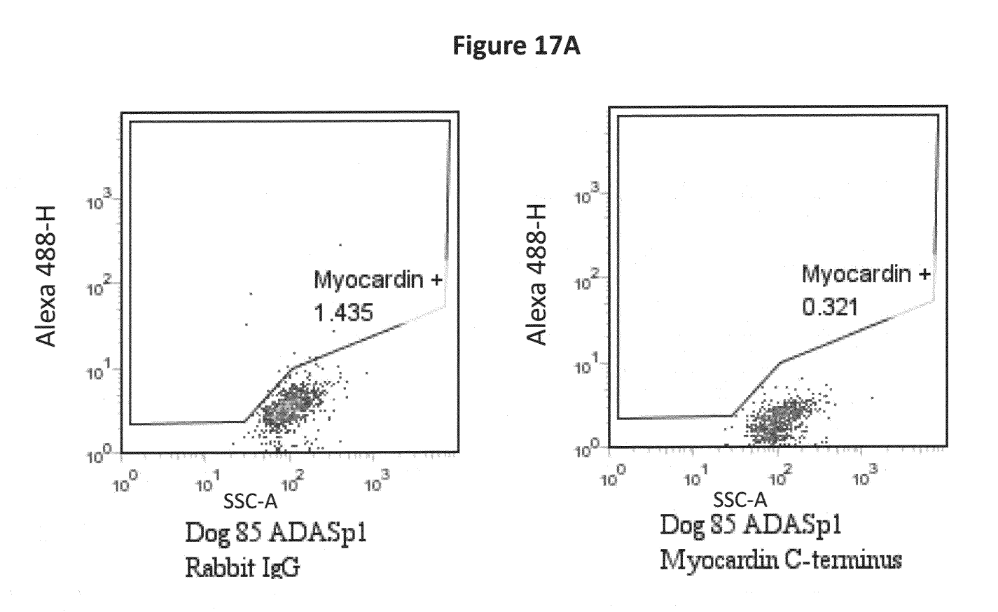

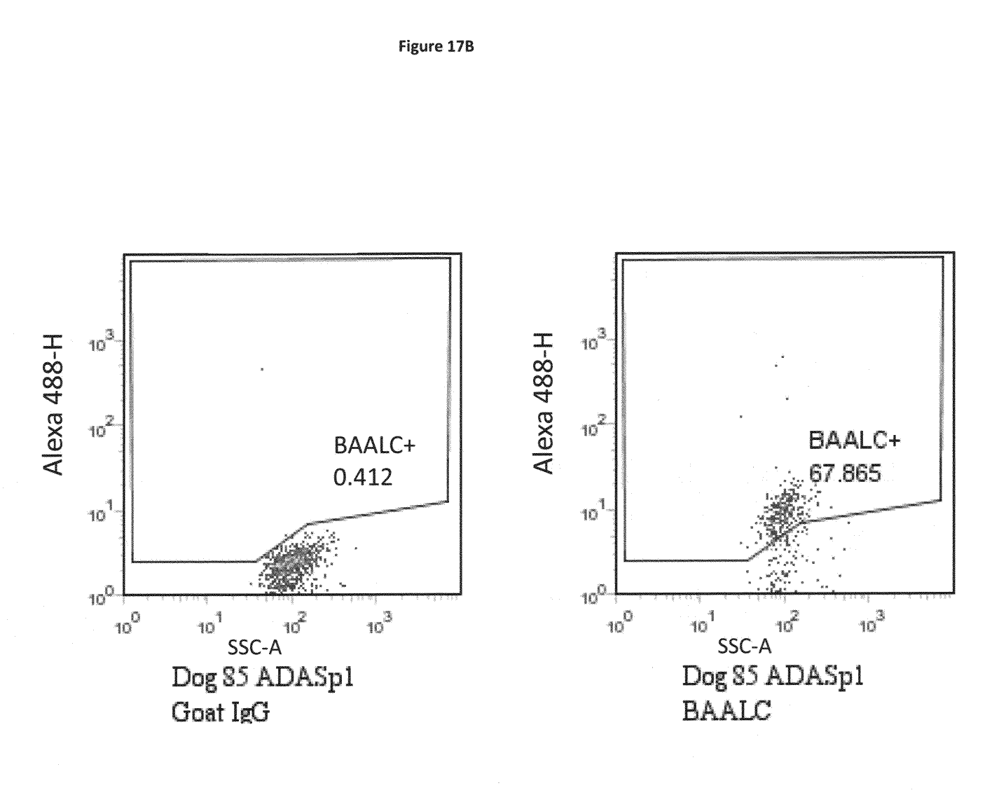

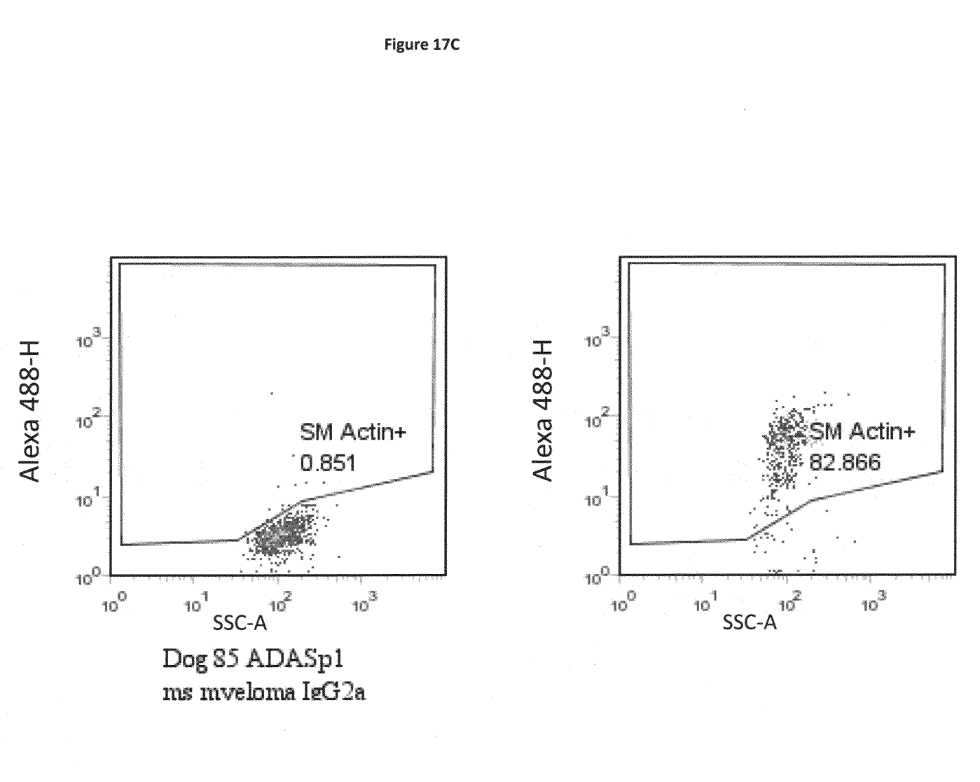

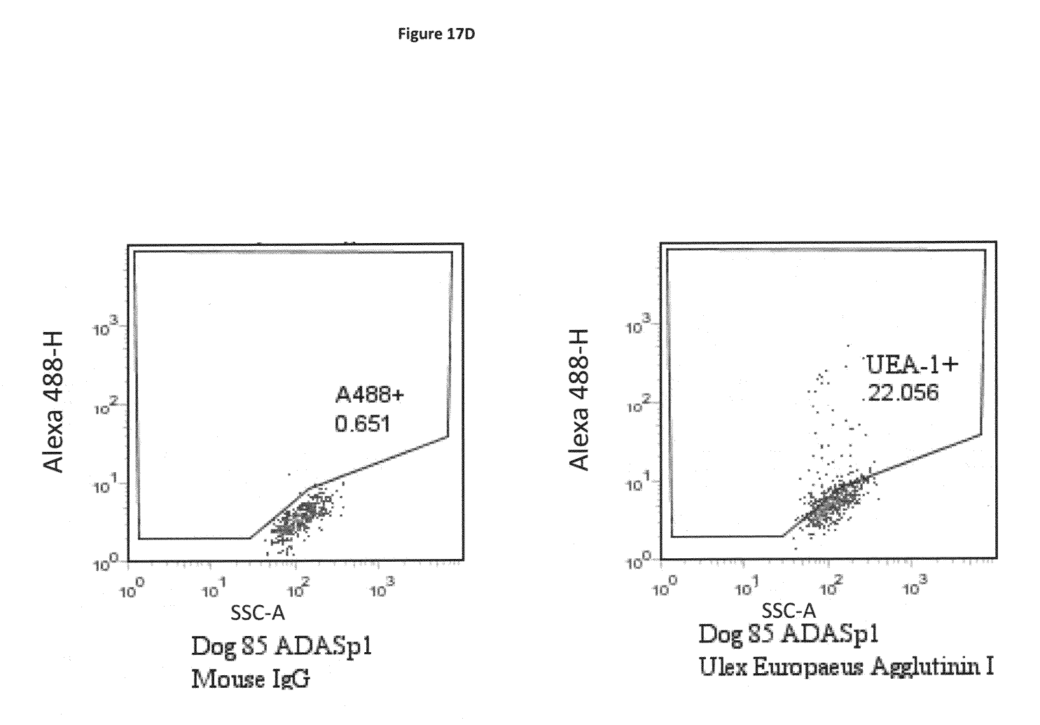

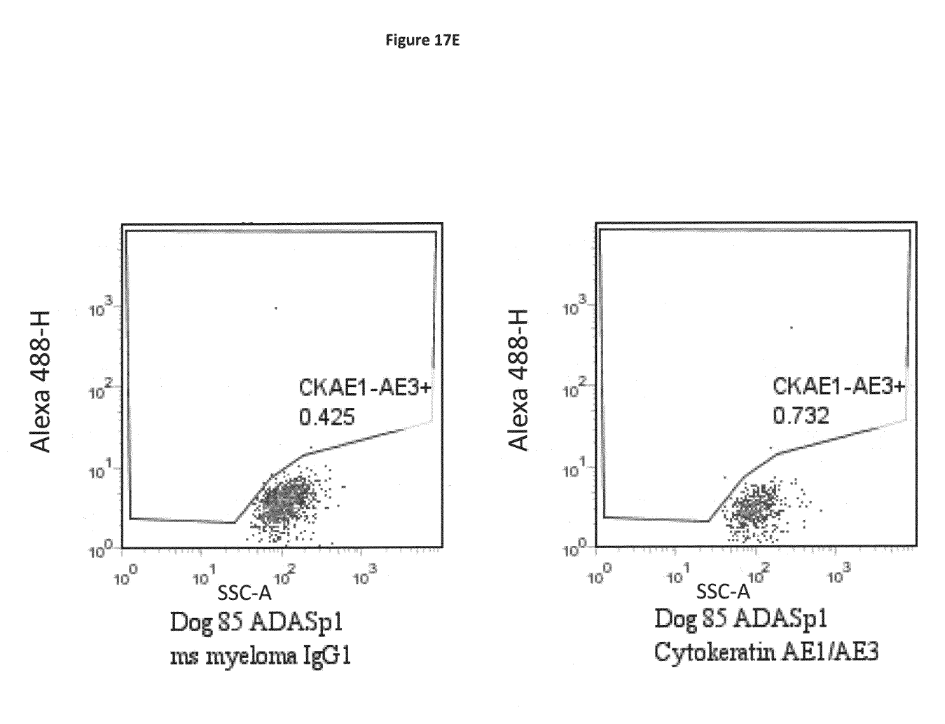

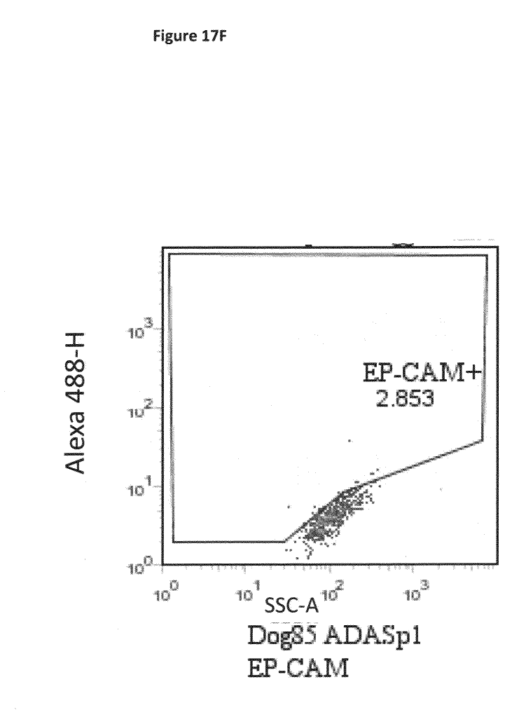

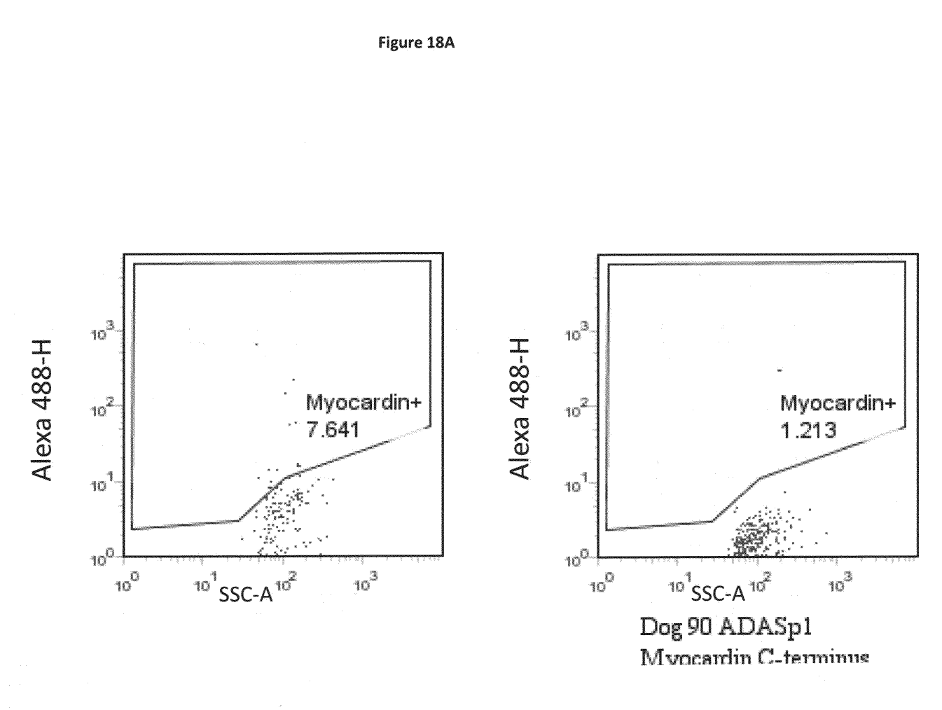

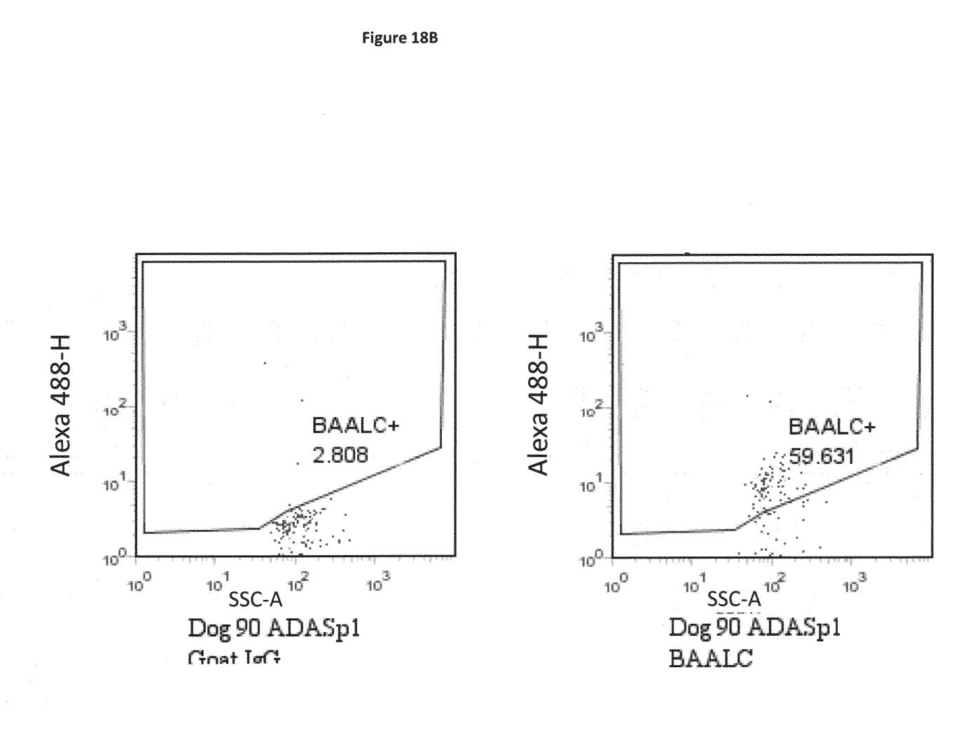

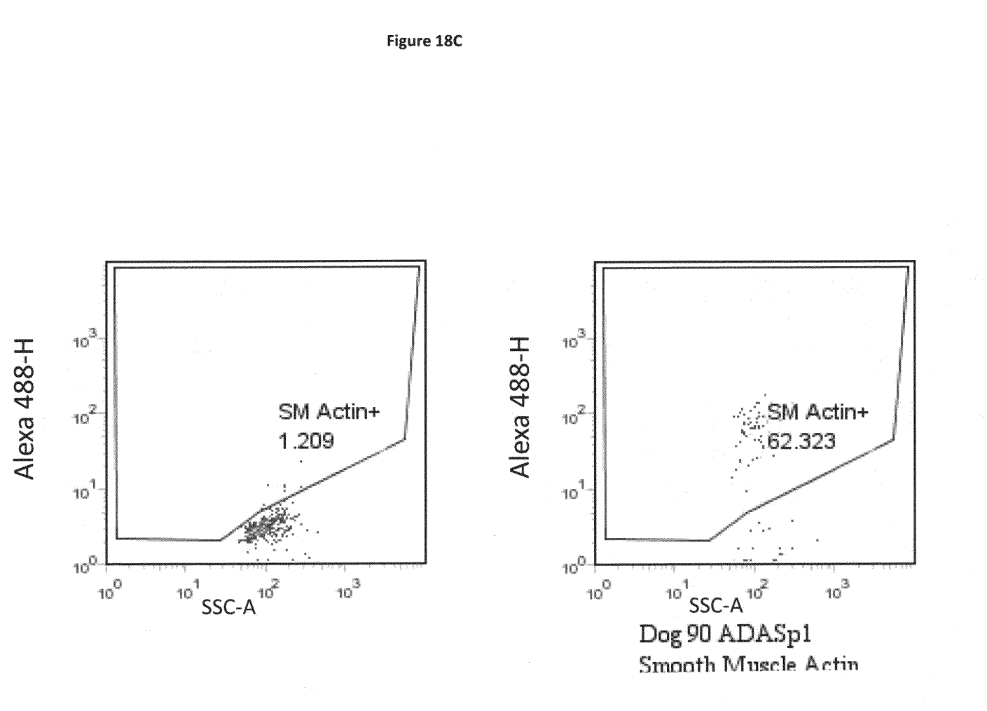

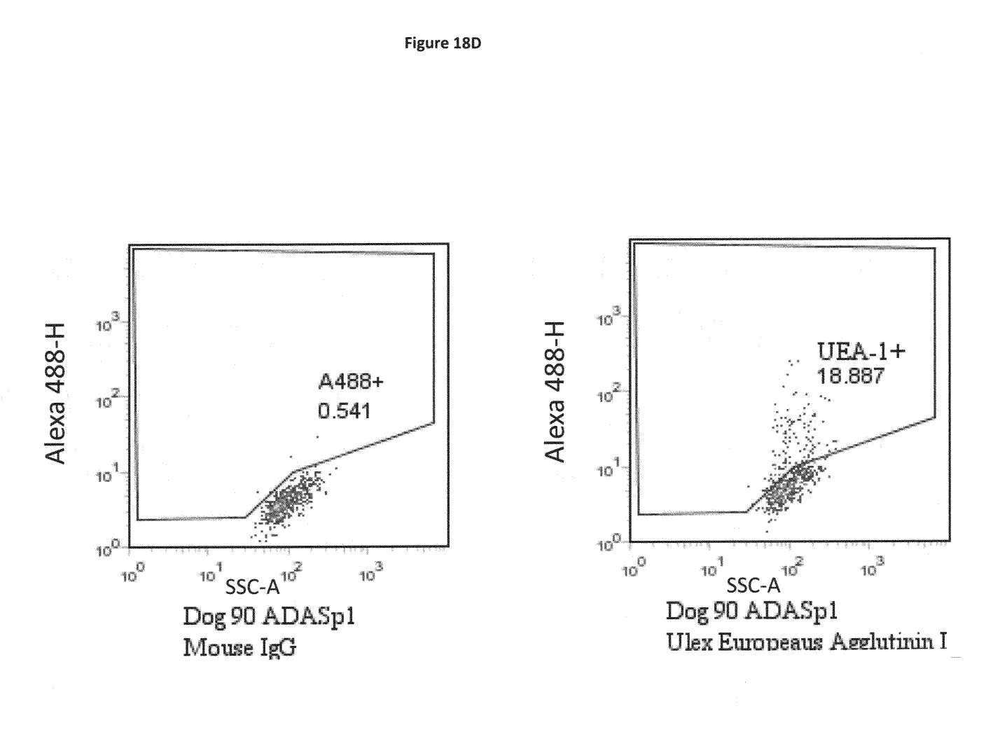

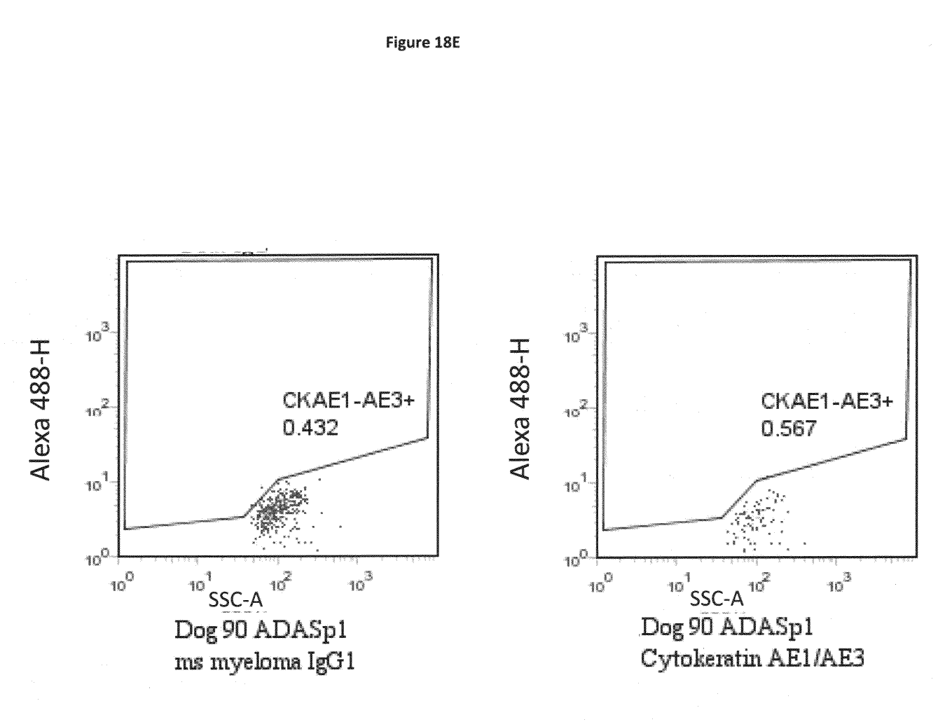

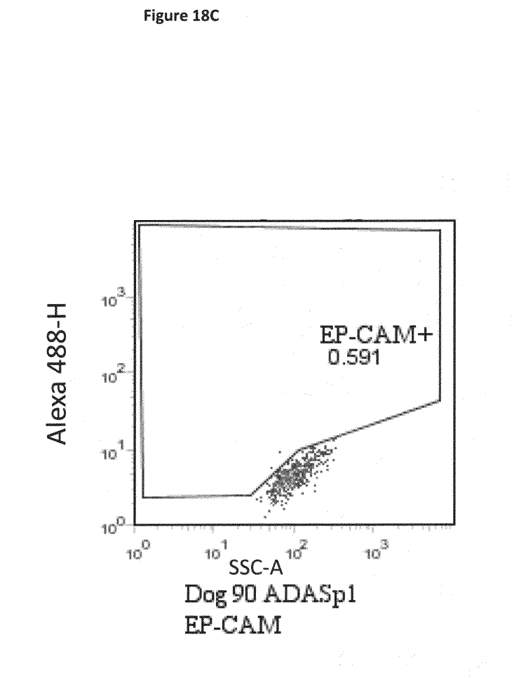

FIGS. 17 and 18 depict further FACS antigenic expression analyses of canine omentum-derived cells from two different animals by looking at both smooth muscle cell markers, as well as epithelial and endothelial antigenic markers. Staining was carried out on omental-derived cells at 1 ug/ml of primary & secondary antibodies. As summarized in Table 2.1 below, canine omental derived cells are positive for smooth muscle cell markers and negative for epithelial and endothelial cell markers.

TABLE 2.1

| | | | | % | % |
|---|---|---|---|---|---|
| | | | | Positive | Positive |
| Marker | Expressed In | Type | Source | Dog 85 | Dog 90 |
| Myocardin | Differentiating | Polyclonal | Rabbit | negative | negative |
| C-terminus | Smooth Muscle | | | | |
| | Cells- human, | | | | |
| | rat, mice | | | | |
| BAALC | Developing | Polyclonal | Goat | 67.45 | 56.80 |
| | and mature | | | | |
| | muscle | | | | |
| | cells - mice | | | | |
| Smooth | Smooth | Monoclonal | Mouse | 82.02 | 61.11 |
| Muscle | muscle | | | | |
| Alpha Actin | cells- Human, | | | | |
| | dog | | | | |
| Cytokeratin | Epithelial | Monoclonal | mouse | negative | negative |
| AE1/AE3 | origin, | | | | |
| | urothelialium- | | | | |
| | human, dog | | | | |
| Epithelial | Epithelial | Monoclonal | Mouse | Neg- | negative |
| adhesion | cells- human | | | ative | |
| Molecule | | | | | |
| EP-CAM | | | | | |
| Ulex | Endothelial | Monoclonal | Mouse | 21.41 | 18.35 |
| Europaeus | cells- human | | | | |
| Agglutinin | sheep | | | | |
| 1-UEA 1 | | | | | |

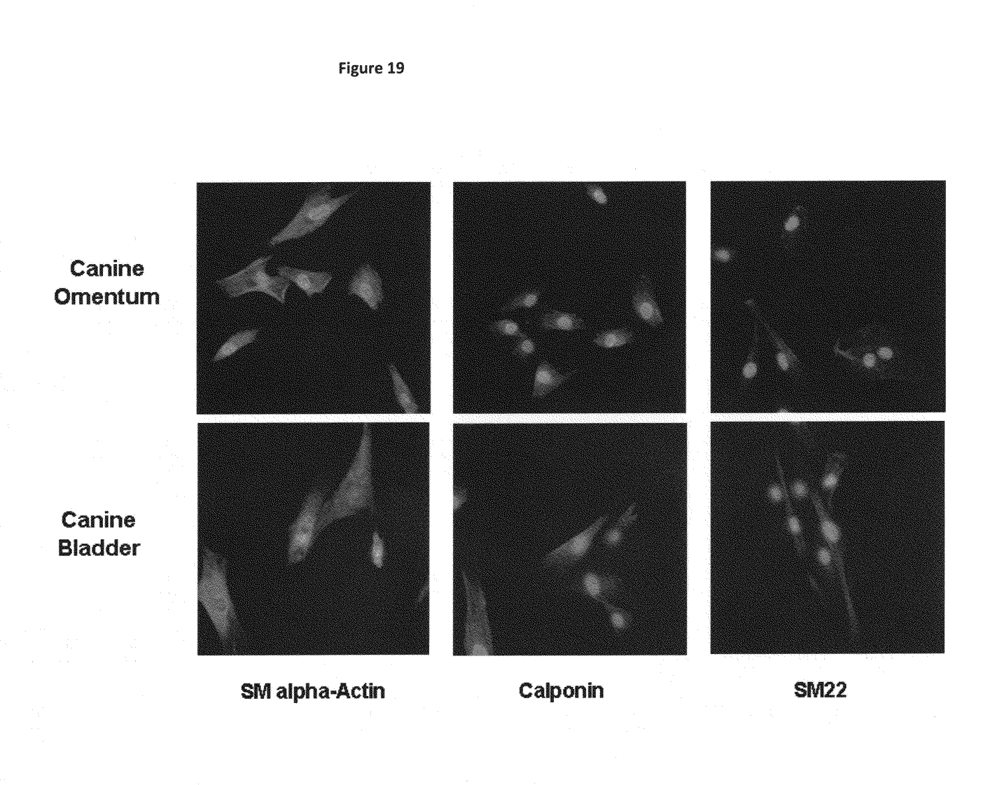

Immuno-fluorescence analysis. FIGS. 19-21 further demonstrate the smooth muscle cell-like phenotype of omental and bladder-derived smooth muscle cellby immuno-fluorescence analysis of varioius smooth muscle cell markers and comparison with endothelial and epithelial cell markers. Briefly, Cells were fixed with 2% paraformaldehyde (Sigma) and blocked with 10% horse serum (Gibco)/0.2% Triton X-100 (Sigma)/ D-PBS (Gibco). Primary antibodies were added and plates incubated overnight at 4° C. Cells were washed 3 times with 2% horse serum/0.2% Triton X-100/D-PBS, then incubated with 1:500 dilution of secondary antibodies: goat anti-mouse IgG1 and goat anti-mouse IgG2a for 1-3 hours at room temperature. Nuclei were counterstained with Hoechst 33342. Mouse IgG1 (Invitrogen) and mouse IgG2a (Invitrogen) isotype controls served as negative controls (not shown). Images were captured with a Leica DMI4000B epi-fluorescence microscope running Simple PCI software.

FIG. 19 depicts immunostaining of calponin, smooth muscle (SM) alpha-actin, and transgelin (SM22) expression in canine omental and bladder-derived cells. Green fluorescence confirms that omental-derived cells express these three smooth muscle-specific proteins at levels comparable to that expressed by bladder smooth muscle cells.

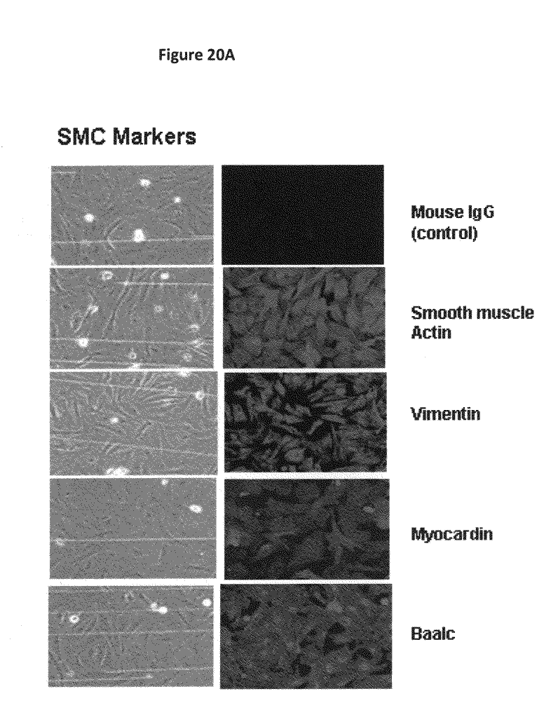

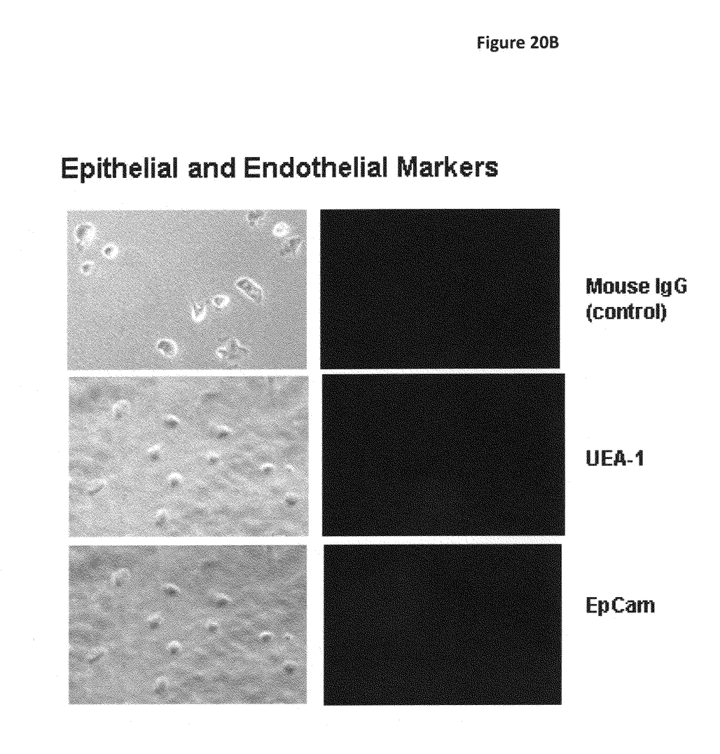

FIGS. 20A and 20B depict immuofluorescence analysis of canine omentum-derived cells to show that these cells are positive for the smooth muscle cell markers (smooth muscle actin, vimentin, myocardin, and baalc (brain and acute leukemia cytoplasmic protein)), and negative for epithelial and endothelial cell markers (UEA-1 and EpCam). On the left side of each panel are control images verifying the cellular content of the image field. The images on the right side of each panel displaying green fluorescence confirm that omental-derived cells express smooth muscle-specific proteins.

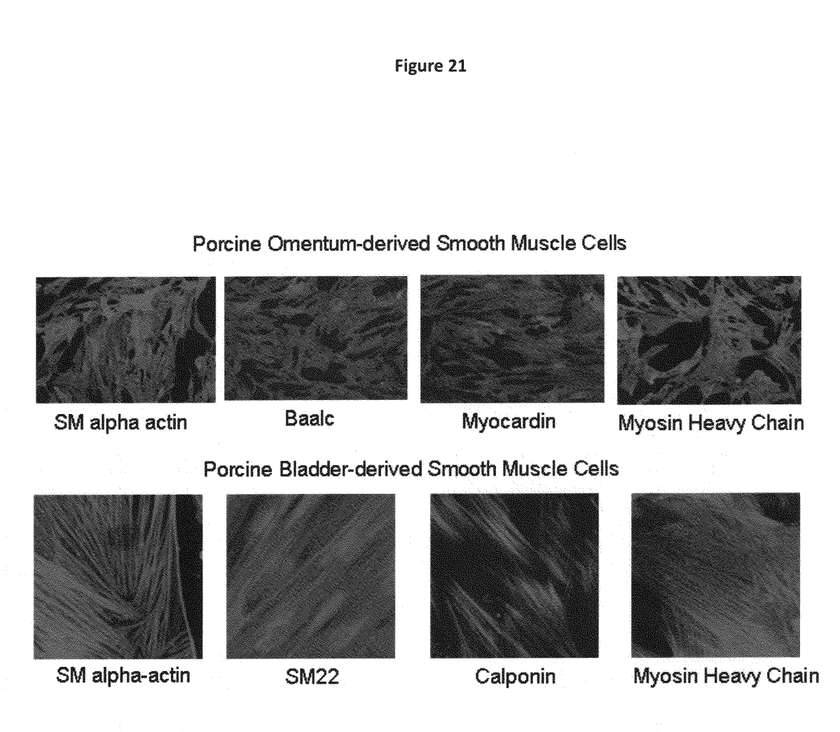

FIG. 21 depicts an immunostaining analysis showing that porcine omentum-derived cells are also positive for smooth muscle cell markers by immunofluorescence and similar to bladder-derived cells. This additional immuofluorescent data shows expression of smooth muscle actin, baalc, myocardin, and myosin heavy chain in SMCs derived from porcine omentum and porcine bladder.

A summary of the cell phenotype analysis by antigenic marker expression is provided in Table 2.2 below, showing the comparison of canine and porcine omentum-derived cells to human bladder smooth muscle cells. Together, these data demonstrate that canine omental-derived cells (extracted from 3 different dogs, cultured in 3 different medium types, at passages 2 and 3) and porcine omental-derived cells (PODS, cultured in 2 different medium types, at final passage 5) are positive for 6 different smooth muscle markers, and negative for epithelial and myofibroblast cell markers. These same markers are expressed on the human bladder smooth muscle cells used as a control (Hu1022 SMC, passage 5). Further supports the notion that omentum may be an alternate source of SMCs. Accordingly, these immunohistochemical data further support the finding that smooth muscle cells are being isolated from omental tissue.

TABLE 2.2

| | Cell Phenotype by Antigenic Marker Expression | | | | | | | |
|---|---|---|---|---|---|---|---|---|
| | | SM- | | | | | Myofibro- | CKAE1/ |
| Sample | Desmin | Actin | Myosin | Calponin | Myocardin | Vimentin | blast | AE3 |
| Human Bladder SMC | | | | | | | | |
| Hu1022 | (+) | (+) | (+) | (+) | (+) | (+) | (−) | (−) |
| SMCp5 | | | | | | | | 30% |
| Canine Omentum-derived cells | | | | | | | | |
| Dog 1083 p3 | (+) | (+) | (+) | (+) | (+) | (+) | (+) | (−) |
| SMC | 15% | | | | | | | |
| Dog 1089 p3 | (+) | (+) | (−) | (+) | (+) | (+) | (−) | (−) |
| SMC | | | | | | | | |
| Dog 1083 p3 | (+) | (+) | (−) | (+) | (+) | (+) | (−) | (−) |
| PM-1 | 5-10% | | | | | | | |
| Dog 1089 p2 | (−) | (+) | (−) | (+) | (+) | (+) | (−) | (−) |
| MCDB + Hep | | | | | | | | |
| Dog 1089 p3 | (+) | (+) | (−) | (+) | (+) | (+) | (−) | (−) |
| PM-1 | | | | | | | | |
| Dog 1090 p3 | (+) | (+) | (−) | (+) | (+) | (+) | (−) | (−) |
| SMC | | | | | | | | |
| Dog 1090 p3 | (+) | (+) | (−) | (+) | (+) | (+) | (−) | (−) |
| PM-1 | | | | | | | | |
| Dog 1090 p2 | (+) | (+) | (−) | (+) | (+) | (+) | (−) | (−) |
| MCDB + Hep | 5-10% | | | | | | | |
| Porcine Omentum-derived cells | | | | | | | | |
| PODS p5 | (+) | (+) | (+) | (+) | (+) | (+) | (−) | (−) |
| SMC | | | | | | | | |
| PODS p5 | (+) | (+) | (+) | (+) | (+) | (+) | (−) | (−) |
| PM-1 | | | | | | | | |

Polymerase Chain Reaction (PCR)-based gene expression analysis. Endothelial and smooth muscle cell gene expression analysis was assessed by PCR. Sample RNA was isolated with the RNeasy Plus Mini Kit (Qiagen) according to the manufacturer's instructions. cDNA was generated from 2 ug of RNA using the SuperScript VILO cDNA Synthesis Kit (Invitrogen) according to the manufacturer's instructions. Following cDNA synthesis, each sample was diluted 1:10 with distilled water. Quantitative reverse-transcription polymerase chain reaction (qRT-PCR) was setup using the TaqMan primers and probes listed in Table 2.2 (Applied Biosystems, Carlsbad, Calif.) and a reaction mixture comprising lOul master mix (2×), 1 ul primer/probe, and 9 ul cDNA (diluted 1:10). Reactions were carried out in an ABI 7300 real time thermal cycler using default cycling parameters. Analysis of PCR data was performed using the method of Relative Quantitation (RQ) by Comparative Ct. Relative Quantity is the amount of target normalized to an endogenous reference and relative to a calibrator and is given by the equation: 2^{-ΔΔCT} were ΔΔCT=ΔCT_{Test}−ΔCT_{calibrator}. The endogenous reference (internal control) was 18S rRNA. Human Aortic Endothelial cells (Human AEC) were used as a positive control for expression of these genes. Table 2.3 lists the genes tested for expression in the PCR experiments.

TABLE 2.3

| Gene | Abbrv. | Marker | TaqMan Cat # |
|---|---|---|---|
| Smooth Muscle | ACTA2/SMAA | Smooth Muscle | Hs00909449_m1 |
| Alpha Actin | | | |
| Transgelin/SM22 | SM22 | Smooth Muscle | Hs00162558_m1 |
| Myocardin | MYOCD | Smooth Muscle | Hs00538076_m1 |
| Smooth Muscle | MYH11/ | Smooth Muscle | Hs00224610_m1 |
| Myosin Heavy | SMMHC | | |
| Chain | | | |
| Calponin | CNN1 | Smooth Muscle | Hs00154543_m1 |
| Cadherin 5 | CDH5/VECAD | Endothelial | Hs00174344_m1 |
| vonWillebrand | vWF | Endothelial | Hs00169795_m1 |
| Factor | | | |
| Platelet/Endothelial | PECAM1 | Endothelial | Hs00169777_m1 |
| Cell Adhesion | | | |
| Molecule | | | |
| FMS-Related | FLT1/VEGFR | Endothelial | Hs01052936_m1 |
| Tyrosine Kinase 1 | | | |
| Kinase Insert | KDR/FLK1 | Endothelial | Hs00176676_m1 |
| Domain Receptor | | | |
| Tyrosine Kinase | TEK | Endothelial | Hs00945155_m1 |
| 18S Ribosomal | 18S | Endogenous | 4319413E |
| RNA | | Control | |

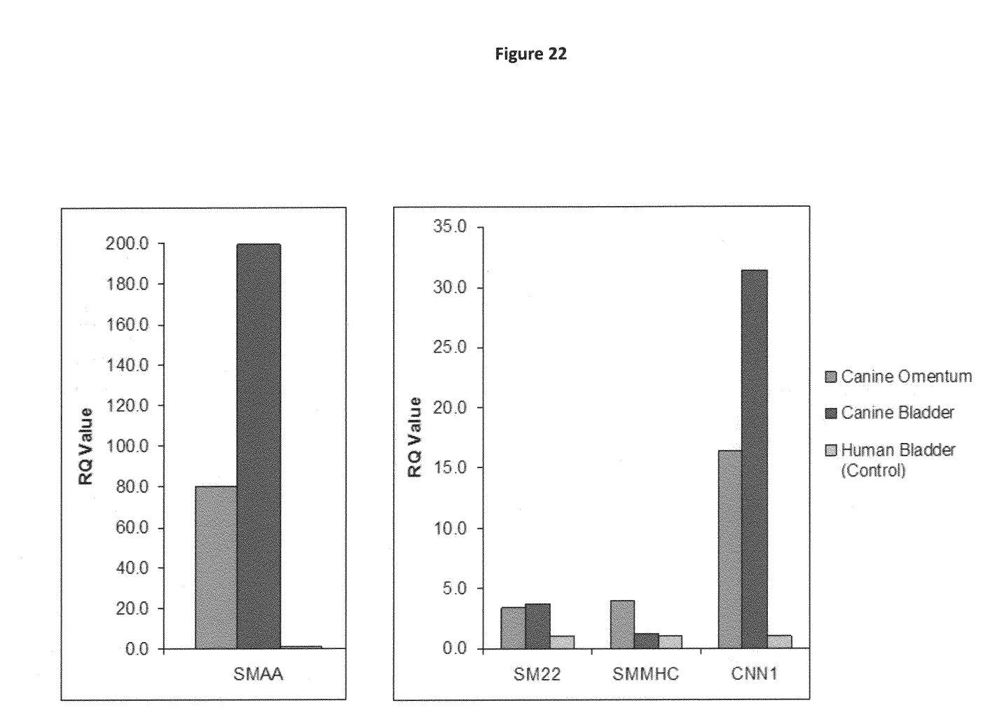

FIG. 22 illustrates gene expression levels of the smooth muscle cell markers actin (cells at passage 1), SM22, myosin heavy chain and calponin between canine omental-derived cells, canine bladder-derived smooth muscle cells, and human bladder cells as a control. Both omental and bladder-derived smooth muscle cells show elevated expression of smooth muscle cell genes as compared to the human bladder control.

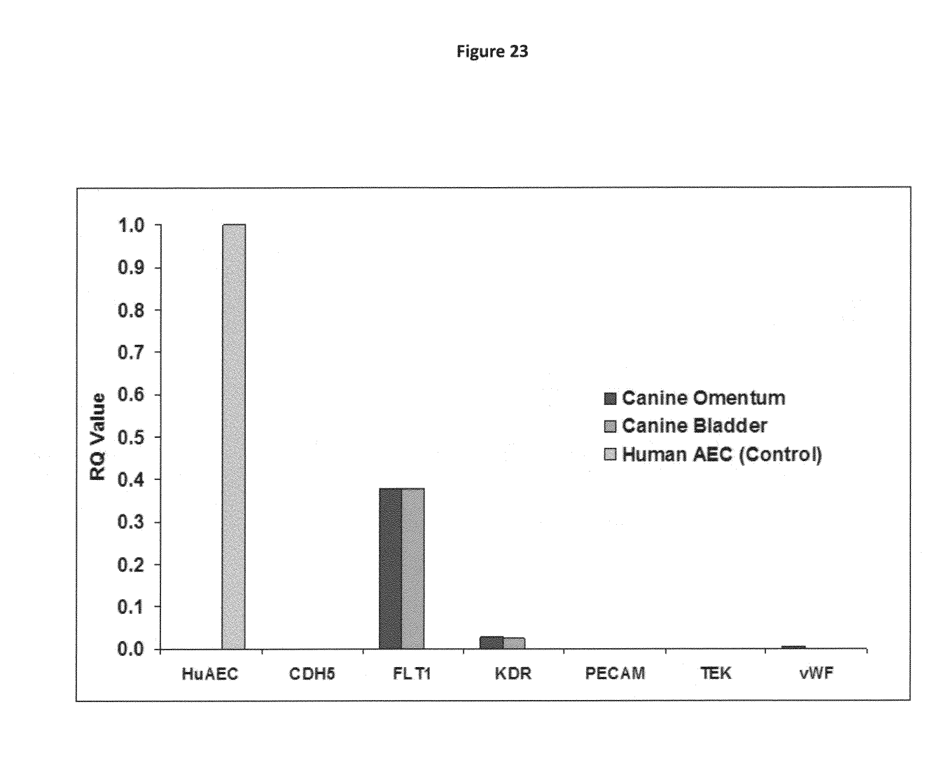

FIG. 23 illustrates gene expression levels of the endothelial cell markers CDH5, FLT1, KDR, PECAM, TEK, and vWF between canine omental-derived cells, canine bladder-derived smooth muscle cells, and human bladder cells as a control. With the exception of FLT1 and to a much lesser degree KDR, the remaining endothelial genes were not expressed by bladder smooth muscle cells or omental derived cells. Where present, omental smooth muscle cells and bladder smooth muscle cells show the same limited expression of endothelial cell markers.

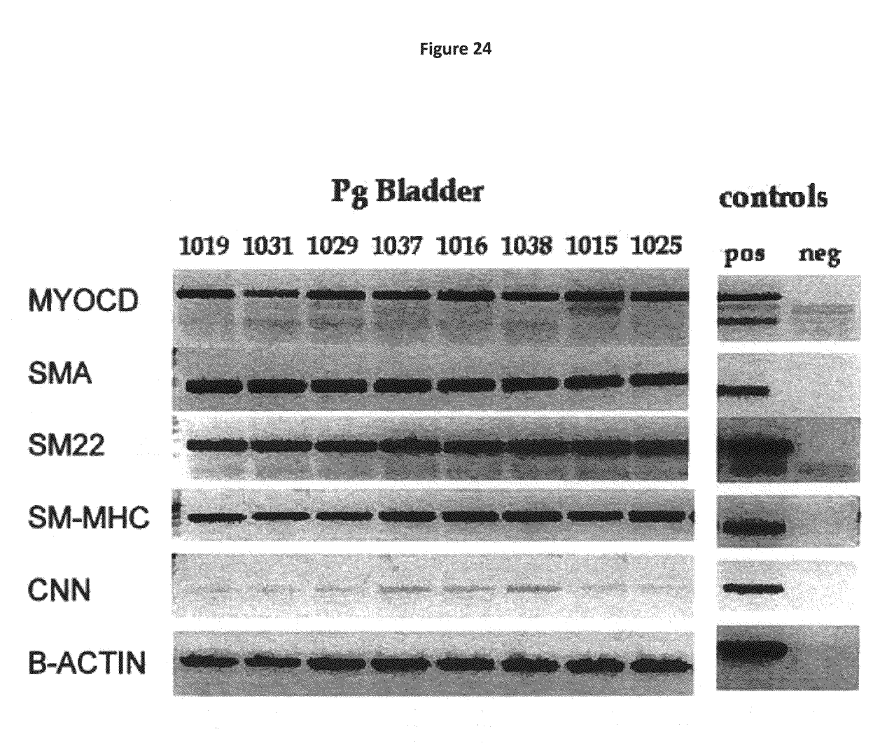

FIG. 24 shows the gene expression levels of smooth muscle cell markers (MYOCD, SMA, SM22, SM-MHC, CNN, B-ACTIN) for porcine-derived cells. The qRT-PCR results, analyzed qualitatively by gel electrophoresis, show that porcine omentum-derived smooth muscle cell gene expression is similar to that of porcine bladder-derived smooth muscle cells.

Gel contraction functional assay. A functional assay for smooth muscle cells commonly used in the art is the ability to contract when embedded in a collagen gel. Smooth muscle cells from canine bladder or canine omentum were suspended at 500,000 cells/mL in a solution containing 2 to 3 mg/mL rat tail collagen I (BD Biosciences, San Jose, Calif., USA). Concentrated MEM (Invitrogen, Carlsbad, Calif., USA) supplemented with 1.8 mg/mL NaHCO3 (Sigma, St Louis, Mo., USA) and 2.3mg/mL L-glutamine (Invitrogen) was used as a diluent and pH adjusted with 3.7 mg/mL HEPES (Invitrogen) to permit collagen polymerization. Negative control hydrogels were supplemented with 5 uM EDTA (Invitrogen) to inhibit Ca2+-dependent cellular contraction. For each replicate, 250 uL of the cell suspension was dispensed into a single well of a 48-well plate. Once polymerized, the collagen gels were gently loosened from the well plate to reduce friction or adhesion that can prevent complete contraction. Serum-free DMEM (250 uL) was added to the top of each gel in the well plate and incubated at 37oC, in a humidified, 5% CO2 containing atmosphere. All gels were imaged using a Molecular Imager ChemiDoc XRS System (BIO-RAD, Hercules, Calif., USA) at 0 hr, 24 hr, and 48 hr time points. Images were measured with ImageJ software version 1.40 g and expressed in pixel units. Well plate collagen gel diameters were calculated from the surface area to improve accuracy. Reduction in gel diameter indicates that the gels contracted.

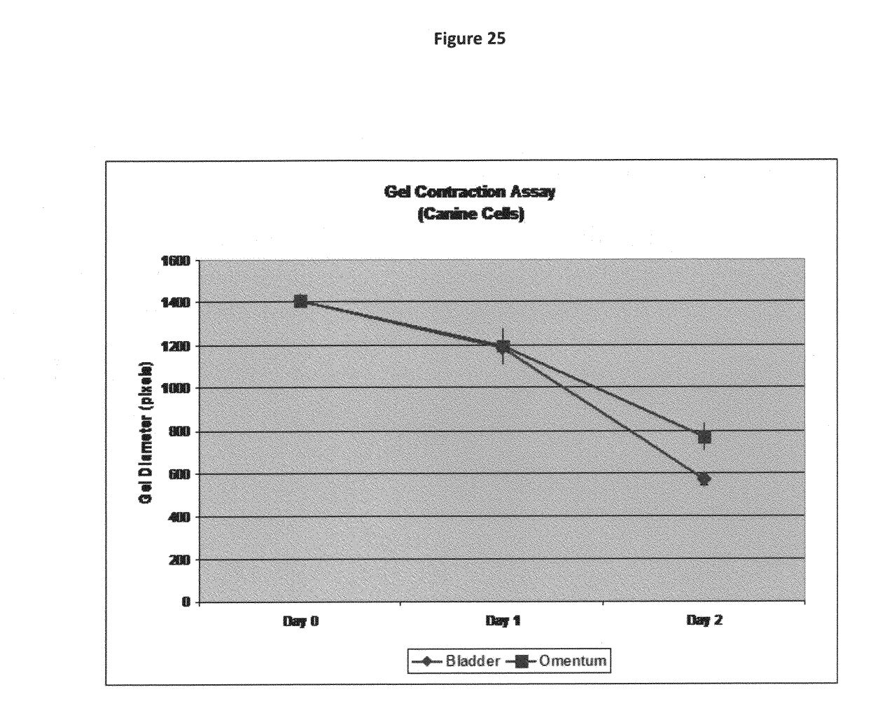

FIG. 25 shows that omental and bladder derived smooth muscle cells have a demonstrated ability to contract, which is a characteristic function of smooth muscle cells.

### Example 3

### Omentum-Derived Smooth Muscle Cells Seeded in Neo-Urinary Conduit Scaffold

A neo-urinary conduit as described herein is composed of biodegradable scaffold shaped in the form of a tube (conduit) and smooth muscle cells seeded on the scaffold. For this study smooth muscle cell derived from Omentum tissue were seeded on scaffold material prepared using the same process as for the Neo-Urinary Conduit. Cells inside the scaffold were evaluated for smooth muscle cell characteristics, including the following: cell phenotype by antigenic expression; protein expression; extracellular matrix (ECM) production; and metabolic activity profile.

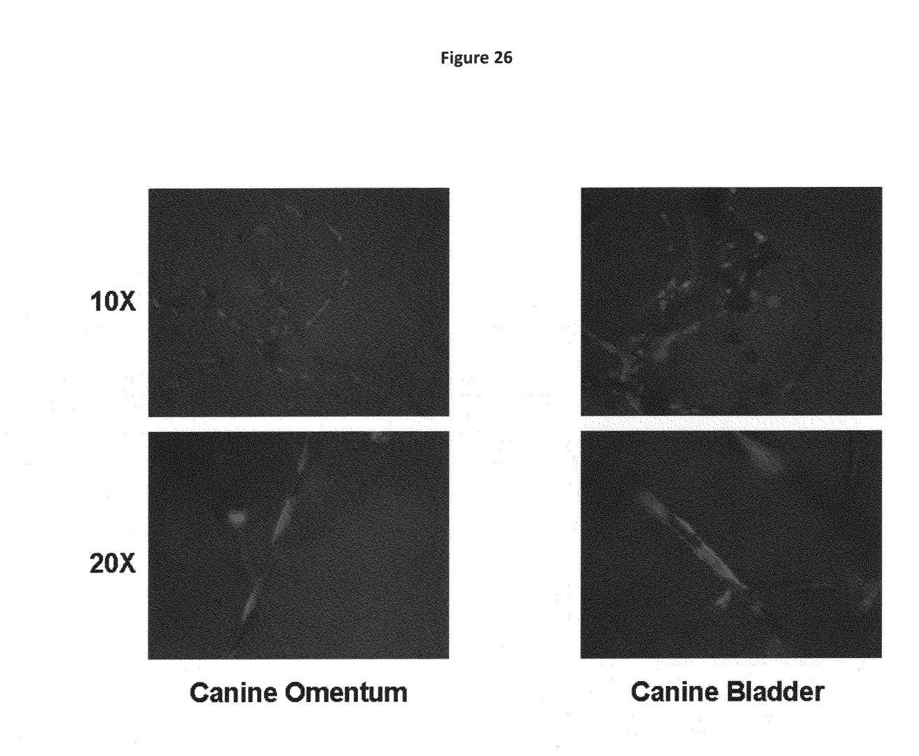

Immmunohistochemical staining. FIG. 26 shows immmunohistochemical staining of bladder and omental derived smooth muscle cells following seeding onto scaffold material. Cell seeded scaffolds were fixed and stained with antibody to smooth muscle alpha-actin as described above. The immunostaining analysis of the omentum-derived smooth muscle cell phenotype inside the scaffold showed expression of smooth muscle alpha-actin. Thus, omental-derived cells retain the smooth muscle cell phenotype inside the scaffold and appear to behave the same as bladder-derived smooth muscle cells when seeded onto scaffold.

MCP1 protein secretion MCP-1 is a normal product of bladder smooth muscle cells and may be used as a marker for potency, identity, and functionality. An ELISA based assay system specific for canine MCP-1 from R&D Systems was employed. Samples were assayed in duplicate and compared to a standard curve to provide estimated MCP-1 levels in construct medium. As illustrated in FIG. 27, omentum-derived smooth muscle cells seeded to the scaffold produce the MCP 1 protein.

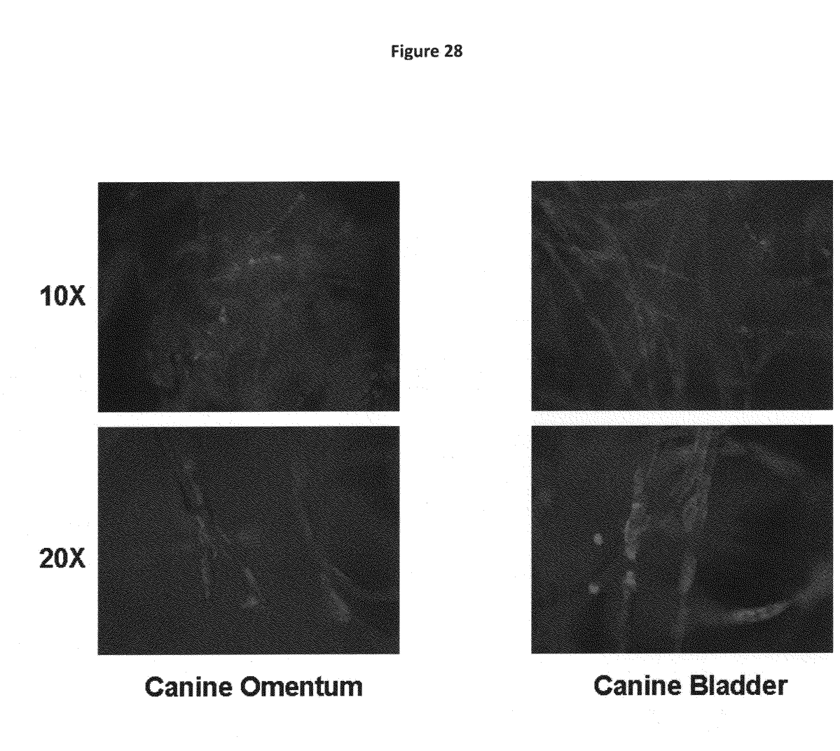

ECM production. To assess the ability of omental-derived cells to produce ECM proteins, immmunohistochemical staining of bladder- and omental-derived smooth muscle cells was carried out following seeding onto scaffold material. Cell seeded scaffolds were fixed and stained with antibody to fibronectin as described above. As depicted in FIG. 28, omental-derived smooth muscle cells synthesize the extracellular matrix material fibronectin, which is important for cell adhesion, migration, growth, and differentiation. Moreover, omental smooth muscle cells again behave similarly to bladder smooth muscle cells when seeded onto a scaffold.

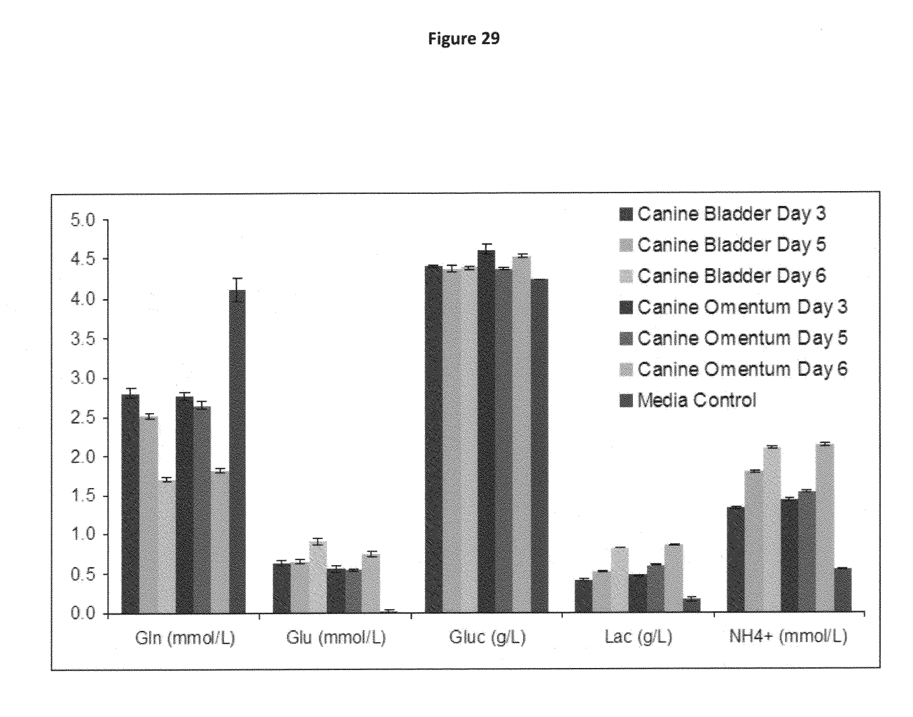

Metabolic profiling. Metabolic profiles for canine bladder-derived smooth muscle cells and omentum-derived cells were further analyzed upon seeding to the scaffold. Medium samples were taken over a time course of 6 days. Samples were analyzed using an automated system (Biolyzer). Briefly, media was added to sample chambers and injected into the machine to measure the levels of various metabolites. As depicted in FIG. 29, the metabolic profiles for canine bladder smooth muscle cells and omentum derived cells are similar with respect to levels of Gln, Glu, Gluc, Lac and NH4+. Thus demonstrating that omentum-derived cells are metabolically active while seeded onto a scaffold.

These studies show that smooth muscle cells can be effectively isolated from omental tissue. Omentum-derived cells have the same characteristics as smooth muscle cells isolated from the bladder. Omentum-derived cells demonstrated smooth muscle cell morphology on isolation and expansion. Phenotypic analysis by antigenic markers was the same as found on bladder smooth muscle cells. Gene expression was similar for omentum and bladder-derived cells. Expression of endothelial cell markers was the same as detected in bladder smooth muscle cells. Also, adipose markers were not detected in cell culture (data not shown), same as for bladder smooth muscle cells. Finally, the omental derived cells also demonstrated a contractile phenotype, similar to bladder-derived cells. Thus, based on these observations, we find that smooth muscle cells have been successfully isolated from canine and porcine omentum.

We also demonstrate that omentum-derived smooth muscle cells behave the same as smooth muscle cells isolated from bladder tissue when seeded on Neo-Urinary Conduit Scaffold, as shown by the following characteristics: antigenic marker expression (smooth muscle alpha-actin); protein expression (MCP-1); ECM production (Fibronectin); metabolism (Glucose uptake, lactate production etc.).

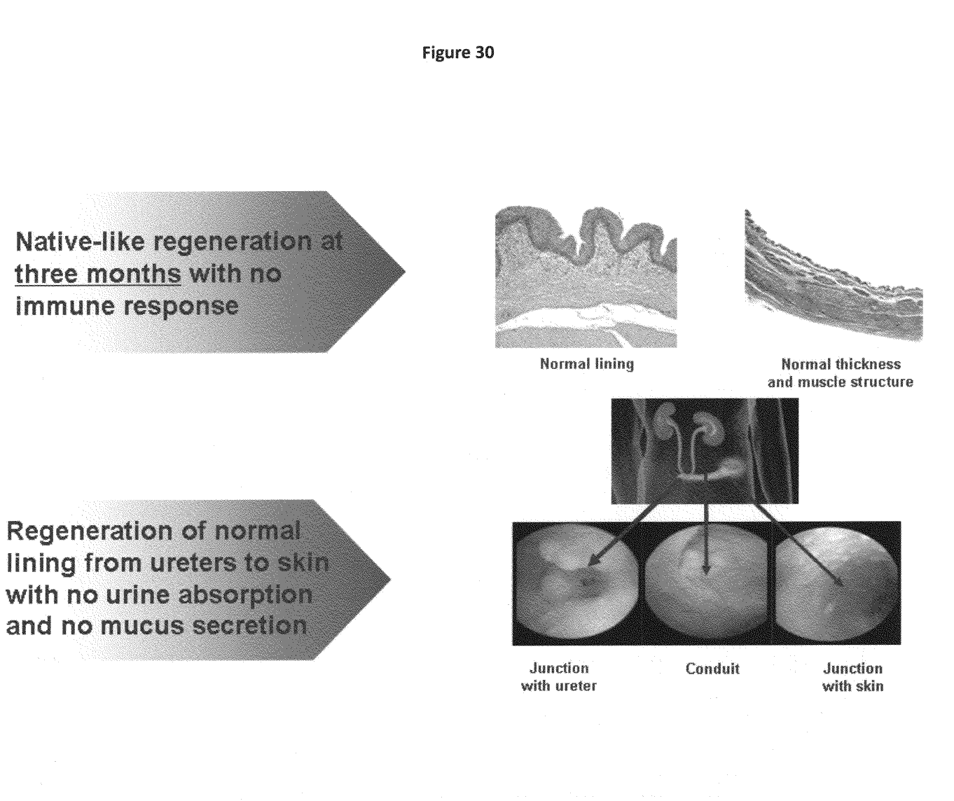

FIG. 30 depicts characteristics of a Neo-Urinary Conduit seeded with another type of alternatively sourced smooth muscle cells (adipose-derived smooth muscle cells) following implantation. A native-like regeneration can be observed at three months without an immune response (A). In addition, a mucosal lining at the ureteral and skin junctions allows water-tight flow of urine (B). There was no evidence of abnormal cell growth or tissue development, urine absorption, mucus secretion, or immune rejection.

These findings suggest that omentum can be used as an alternate source of smooth muscle cells for the production of a Neo-Urinary Conduit.

What is claimed is:

1. An implantable construct comprising: a) a matrix having a first surface, wherein said matrix is shaped to conform to at least a part of a native luminal organ or tissue structure in a subject in need; and b) a peritoneal-derived cell population deposited on or in said first surface of the matrix, said matrix and said cell population forming an implantable construct.

2. An implantable construct comprising: a) a tubular matrix having a first surface, wherein the matrix is shaped to allow the passage of fluid from a native vessel in a subject in need; and b) a peritoneal-derived cell population deposited on or in said first surface of the matrix, said matrix and said cell population forming an implantable construct.

3. The implantable construct of claim 1 or 2, wherein the cell population is a smooth muscle cell (SMC) population.

4. The implantable construct of claim 2, wherein the tubular matrix comprises a first end.

5. The implantable construct of claim 4, wherein the first end is configured to contact the subject's abdominal wall.

6. The implantable construct of claim 5, wherein the first end is configured for anastomosis to an opening in the subject's abdominal wall.

7. The implantable construct of claim 5 or 6, wherein the first end is configured to be exteriorized to the subject's skin.

8. The implantable construct of any one of claims 4 to 6, wherein the tubular matrix further comprises a first side opening for connection to said native vessel.

9. The implantable construct of claim 8, wherein the native vessel is a first ureter.

10. The implantable construct of claim 9, wherein the tubular matrix further comprises a second end for connection to a second ureter.

11. The implantable construct of claim 9, wherein the tubular matrix further comprises a second side opening for connection to a second ureter.

12. The implantable construct of claim 9, which allows passage of urine from the first ureter to the interior of the tubular matrix upon implantation.

13. The implantable construct of claim 10 or 11, which allows passage of urine from the second ureter to the interior of the tubular matrix upon implantation.

14. The implantable construct of claim 12, which allows passage of urine out of the subject upon implantation.

15. The implantable construct of claim 13, which allows passage of urine out of the subject upon implantation.

16. The implantable construct of claim 7, wherein the first end of the tubular matrix forms a stoma external to the subject upon implantation.

17. The implantable construct of claim 16, wherein the first end comprises a stomal end extending through the subject's abdominal wall.

18. The implantable construct of claim 17, wherein the stomal end is connected to the subject's skin.

19. The implantable construct of claim 17 or 18, which forms an epithelialized mucosa at the stomal end upon implantation.

20. The implantable construct of claim 19, wherein the epithelialized mucosa comprises a mucocutaneous region at the stomal end.

21. The implantable construct of claim 20, wherein the epithelialized mucosa comprises a vestibular region adjacent to the mucocutaneous region.

22. The implantable construct of claim 21, wherein the epithelialized mucosa is characterized by an epithelium that first appears in the vestibular region and gradually increases through the mucocutaneous region towards the stomal end.

23. The implantable construct of claim 22, wherein the epithelium is characterized by expression of an epithelial cell marker.

24. The implantable construct of claim 19, wherein the epithelialized mucosa is equivalent to a naturally-occurring mucocutaneous region.

25. The implantable construct of any one of claims 1 to 24, wherein the construct is free of urothelial cells, or is free of any other cell population.

26. A method for the reconstruction, augmentation, or replacement of a laminarly organized luminal organ or tissue structure in a subject in need of such treatment comprising implanting the construct of claim 1 into said subject at the site of said treatment for the formation of said laminarily organized luminal organ or tissue structure.

27. A method of preparing an implantable construct for the reconstruction, augmentation, or replacement of a laminarly organized luminal organ or tissue structure in a subject in need of such treatment comprising
a) providing a matrix having a first surface, wherein said matrix is shaped to conform to at least a part of a native luminal organ or tissue structure in said subject; and
b) depositing a peritoneal-derived cell population on or in said first surface of the matrix to form said implantable construct.

28. The method of claim 27, wherein the implantable construct formed is the construct of claim 1.

29. A method of providing an implantable construct for a defective bladder in a subject in need of such treatment comprising implanting a construct according to claim 2 into the subject.

30. A method of preparing an implantable construct for a defective bladder in a subject in need of such treatment comprising
a) providing a tubular matrix having a first surface, wherein the matrix is shaped to allow the passage of fluid from a native vessel in said subject; and
b) depositing a peritoneal-derived cell population deposited on or in said first surface of the matrix to form said implantable construct.

31. The method of claim 30, wherein the implantable construct formed is the construct of claim 2.
